SEQUENCE LISTING (50 sequences)

SEQ ID NO: 1 - protein, 365 aa, Mus musculus
MGMPLPWALSLLLVLLPQTWGSETRPPLMYHLTAVSNPSTGLPSFWATGWLGPQQYLTYNSLRQEADPCGAWMWENQVSWYWEKETTDLKSKEQLFLEALKTLEKILNGTYTLQGLLGCELASDNSSVPTAVFALNGEEFMKFNPRIGNWTGEWPETEIVANLWMKQPDAARKESEFLLNSCPERLLGHLERGRRNLEWKEPPSMRLKARPGNSGSSVLTCAAFSFYPPELKFRFLRNGLASGSGNCSTGPNGDGSFHAWSLLEVKRGDEHHYQCQVEHEGLAQPLTVDLDSSARSSVPVVGIVLGLLLVVVAIAGGVLLWGRMRSGLPAPWLSLSGDDSGDLLPGGNLPPEAEPQGANAFPATS

SEQ ID NO: 2 - protein, 365 aa, Homo sapiens
MGVPRPQPWALGLLLFLLPGSLGAESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSVLVVGIVIGVLLLTAAAVGGALLWRRMRSGLPAPWISLRGDDTGVLLPTPGEAQDADLKDVNVIPATA

SEQ ID NO: 3 - DNA, 4478 bp, Artificial Sequence
  5i - homology arm
tctggaagagctgtcagtgctcttacccactgagccatctctccacccctccaccccacccccattgtattttctggcacaaagcctctcactgaacctggagctcactggtcagcaaactccaggtatcttcccggcccctcccaccctgtgctgggggctggagagagggctggggagcctgtctggggagagcagacaggacaaccatggccgtgcctctgtgctctggacgctgatggggaatgctaggctagggaagagataggagatttggggatccagctcagttggtagttaatctggcgctgtggtttgtggtgttagttcctgttaacttgagctgagaagatggaactgagagtctgcccgtgagcaggtctgtgggggcattttttttttttttttttttttttgatttctgattgatgcaggaggacccagcacacagagcagtgctgctccagggtaggtggtcctgagttgtgtaagaaagcagatagagcaagccgtggagaggaagccagcaagcagcattcctccatggtctctgcatctgttttttgcctccaagttcttgctggagtttctgccctgacttccagtcacgaagggatgtgaaagatgaaataaaccctctcctctccaagctgcttttgactgtggcccttatcccagcaataggaagcaagccagggcatccccatagtcccagcactcaggagggagaaggttaaggttagtgtcttctgcctctctctttttctaccccctcccctttccccctctcccctccctccatctttgtatgggttccaggggtcaaactcagatcatcaggccttctgtcgcagatgcctttagctgctgagccgtctccatggtccccggaaggggttcaaacacaaattggtgcacagggaggttcatagaaaaagaggcattaagggggcgggcccaagcagggacctgggcttctcaaaggggagtctcacgtaagtgtcttaactgtaaccatacacaggggttgggaggctgccttccccacctcgttcttagttaagtgaagtcagagtgcttcctggagggctctggggaggatggcagctgaagaggtaaactggagactgtagggttgcacaagggaagaaagccagcttcttgccgtggacaaaggttccggactgatttgtaagtttggggtttccatatgggaagaaaaaggttattggtgacagccattgagtggagctgattgctgtggtgttagtattcttgtttgaaatctctctccaccatcacaaaggccacagcaatccctgctaactttttttttttttttttttttttttttttttggtttttcaagacagggtttcctcgaactcagaaatctgcctgcctctacctccgaagtgctgggattaaaggcgtgtgccaccatgcccagcccctgctaacttcattgtggcttgactctcagctgtccgcagactttagactctggtgtgacaggctttccatgaacacttccctgccaatccttctttcttcttccctccttccctccttctctcctcccttcctcccttccttctttccttcttccctccttcctttgacagggtcctttgtcgcccagcctggttttaaactttctatgtaattgaggatgaccttgaactctgttgtcctgcgtgctagacaagtagatttacttcattaaatgaaccaaagcccccagacctcatatggtttttgctgttgtggaaacctgactcccgctcactgtgtagatcaggctagtcttgagcacacagggatctgcctacctctgtgtcctgagtgctaaggttaaaggcgtgagcggccactcctgttctttttcagttttacttcctggatttgcttggttctctgtgtccctgcgtgttgagtacagtgccctctgaagccggtagctcccccgtagctggagttacagatggttgtgagcatcgtgtggatgctggcagccaaactcaggtcttctacaagagcagccagtgtttttttttgttgttgttgttgtttggtttttcaagacagagtttctctacgtgaccctggctgtcctggaactctctctatagaccaggctggcctcgaactcagagattcacctgcctctgcctcctgagtgctgggactaaaggtgtgagccaccaccagcacagacagtgtctcttaatcacctagccatctctctagccacagacgcagctttttacaggggaggctagcatcagaagatcaggactcaggtcctcattcttggcaggcaaacacttactgactgagccctctccccagtccgtaggaggcctgcaagcccagcacttaagagctggaggctgggcttcaggagttcatggtcgacctgggctgcatgagttcctgtcctgacacatctaccctacccctctgctgcctccacgctaccaccaccaccaccattattacacacgcacacacttgtacacacacttgatatagaccagagggagagggaggtcaaggcaggtaggagtggttgactttctattctctcttaaaaaggttttttttttaatttatttattggttatatgtaagtacactgtagctgtcttcagacactccagaagagggaatcagatctcgttacggatggttgtgagccaccatgtggttgctgggatttgaactccagaccttcagaagagcagtcgggtgctcttacccactgagccatctcaccagcccttaaaaaggttttttagatttatttattttattcatgcctgtgtgtatacacgtgccccacatccctgcctggtgttcaatgagaaattgtcagaccccctagaactggagttatgggcagctgtgagccaccatgtgggtgccgggaaccaaactcggatcctctgcaagagcagcaggtgctcttaactgctgagctgtctcgtcagctccagactttccagactcttgcttctgtctttcaagtgtgtgggttccaggcctgccccatcacaccggttttgcccatctacttcagcctgttggtccatctgctgaccaccagtcgaccactcacggctctgtcagtctccctctctcggctgtttatattccctgccgcccaggccggccatgaacccctggcctcccggcctccatccttagtgctggaattacaggcatgtgctgccgcccaccccctttcttctttgctgtctatctaccctgtcaacctacctgtctgtcagtgtctctctgtgtgtctgtgtgttttttctggcaagatcttggggtgggcagcagggtgggagacaggggccaaaggcaaaggctctcatcacagagtgtgtaggcagggttggggccacctcaagtgctggcacatggaagaaatcagagcaggcttacagctgggagtgggctgctgccaaaccttgtgtgttaggcaaccatgccatgaccaagccacacccacagcccacaaatcctggggtgtcttaaagtcacattcagcgtgcaggcctttccagggctgcgagtctactccaaaaatgctccaaacgagtaaacatagaagctgttttgggaggcaggcctaatgacgtttgccccgggaggcttccaaccagatacagcaggaacttcaagagaggctggacagaggcccaaggggtgtgcttaggagctagtgggtggagttggatgccctcagagttctccagtcctaactgtgtacagacaggatgtaagagaagaactggaggctctaagcagaggatccatcggctgcaggcagagggaagagggcctctgtgaggaacaggctgagcgtcagaggaggaggcccaggcctggttctctaggtatgagggaccagcaccccaagaggaaagagggttggggcctggagctccaggtctgagggaggagtgttatgacctgagggggagagaggaggctggggtctgaggaaggagagcaggaccccgggcacctgagtctgagggatgaagccggagcctagggttttgggtttgaggaaagatcttggaactggactcctggatctgagggaggtggaactggactcctggcttgggtttgagggctcctgaggctgaaatctgagcccctgggaatgagggaggaggagaaggcctggatcctctggtctgagggaggagaaaaaggaaataacagaagtctgagacaaattttggtctgtctagctctgtaattaattaactaaagtggatcaaatgagaaggtgaaagttcacagaggaacactcctgtctgtcgtcttggactgggtctccatcccaccatccagcgtcctggtctacgaagagtccacagggaccttgtgaagaatcaacaaggcggggtccagaggagtcacgtgtcccttccactccgggtcaccctgtcgga

SEQ ID NO: 4 - DNA, 6610 bp, Artificial Sequence
  3i -homology arm
ctagatctccacagccatgctcaaagcccacccagcatcagtacaacccctctcgcctatcccagtggatcttctttaagataaggtctcatgtagcccaggctagctctcacgtagctcaagctagccatgagctcatcgtgtagctgaggatggtcttggattcctgatcctcttagcccattccaagtgctgatttgacttaagtgtgttgccacacccagctaattgttcttgtttgtgagacagggtctcaggctgactttgaactcagagagatctcttgagtgctgggacaaaaggagtgtgctgctgtaccaggctgctttttctttttctttttctttttttgttgttgttatagttagacagactctcccaatgtagaccacgtgacctcaaacttgtcatcctcttggtctcagcctcccaagtgctaggattttccatgtctttttcttcttgttaacgtctaagagttctttatatattgtggacccaaatcttcaccaaataagtgtttcaaacacactttccttgactgttttcctttcattcttttaaagttatcttcaaatctttttgttttgttttttcgagacagggtttctctgtgtagccctggctgtcctggaactcactttgtagaccaggctggcttcgaactcagaaatccgcctgcctctgcctcctgagtgctgggattaaaggtgtgtgccaccacgcccggctttttttttttttttttttaagattatttagttttgtatataagtgttcggattgcatgtatgcctgctgcacaccagaagagggcatcagatcccatttcaggtgcttgtaagatgccatgtggttgctgggacttgaacttaggacctctaaaagagcagccagtgctcttgactgctaagccatctctccagtccccagtcctcttttgagggctgtgtttttgttagtatatcttgcttggaggtcatgcccagcccaggatgacctaggtctctgtgtgtatttggaggagttttatagctttccattctatgtatagatcctcttgcttttctgtctaacaaattgtatgtgaagaaggtctgtcatttctccatccaagtttcagcctgtctggatctgagttgtgtgtgtgctcatgtgggtggagaccagagcacagcctcgagtgttgtatttatgacatctttttctgaaaaacaaatcttcaggctggactcagctcagtggttgagagcacacagggattttatatgtgcacatgtatatttgtatgtgcacgtgagtgtcagaagagggcgttccatcccctgtagctggagttacagttagttataacatgcccagtgtgggtgctggaactgaatttgaggcatctttaagtagcagcaagcccttagtccctgagccacctctccagccccagcccctgtgagatttttactctgtccctctcacttagttggctcagtttgggattgcactcaggtcctttctcttgccaagtaagagctctgccactgagcaatctttaaggttggtttttgattttggtttttggtttttcgagacagggtttctctgtgttggaactcaatctgtagaccaggctggccttgacctcaaagacccatccatctctgcctcctaagctctggagatcagaggcatgcgtcaccgccgaccggctggccatgttttaggttttaaaacttacctttacttatttatctgtgatttagcgtgtgtgagtggtggtggtggaggtggtgtgtgtgtgtgtgtgtgtgtgtgtgtgtgtgtgtgtgtgtttcatatgtgatatatacctgtgctgtgtatggggtgagcatctgtccacatgctcagaagccagagaaggtggctcctctgtctctttccatcgtatgttcttcacccagattctcttgatgagcctgaagctacgcaggcaaccagccaggcccctactggtcctctttcctctgcccctatcctcagcgccagggttacagttatgaagatatgtttaagttttgacataagtgctaagaatttgaacttgggtcctcatgcttgtgtaataaacactttttcatgctgagccattcccctaattatggctaaatagtactccattctagaagcctgcatgctatcttcctttatctgctgagggtgtctacattgtttctgtagtttggctgtttttttgtttgttagagatttatttatttattatatgtaagtatgctgtcttcagacactccagaagaaggcatcagatctcattacggatggttgtgagccaccatgtggttgctaggatttgaactcaggacctttggaaaagacgtcaatgctcttaaccactgagccatctcttcagtcctgtttggctgttttgatgtttctacaatgcaggatagtggccagtgtttccctgaaattttgttttgcttttactatatttactttatttttttatttttggttttttgagacagggtttctctgaatagctcaggctgtcctggaactcactctgtagaccagtatggccttgaactcagaaatctgtctgcctctgcctcccaagttctgggattaaaggcatgtgccaccactgcccatccctaaaattttgataccaacttctggataagtgtccacaaataggatagctaactccattaaacaagaaaaaaaatttttattatgttttttgcttccatgtatgtctgtgcaccatgtgcatgcagtactcacagaggccagaggagggcgtcaggtcccctggtgctggagttacagatggttaggagttgttcaccacatgggtgctctgacttgaacctgagtcctctggaggagctgccactcactacttgactaattttacccgctgagtcacttctctagctctgagctcatttaccatggaaaaccatggtggcctgtgtggggatacatgtctttaatgctagcactccggaggcagggacaggtagtctctgcactacctgcactccccttgacaaccggtccaggttccacgggtactgactacagttgctacattgtgtctatgtgtgattttgttcttcttaacaggcttctggtaccaggtgttaggttgtgttactgtttccttccaaacatacaaacaaggcaggggctctaccactgagccacgccccagcccttcactggggaattcgaggctctaacattgagcataccattgacgctcatttgacttaaaatatttttaaggttacacttattattaaggttacttacttgtgaatgagtgtgcatatgtgtgtgcgtgtgtgcgtgtgtgtgtgtgtgtgtgcgcgtgcgtgcgcgcgcgcgcgcgcgtgcgtgcgtgcgtgtgtgtgtgtgtgtgtgtgtgtgtagataggtcagagaacttggtgagttggatctctccttttgccatgtagattctagggatggaactcaggtcatcaggcttgtccgcaggtgtctttacgtcacgtcaccagccctcttgtatttttctaaacctttttctgactatatttattttattgttaatttctgtggatgtgtgtgtacatatatggtgcaagtgaccacagatgccagaaggaggatcaggtcgtctgcagctggagttagttataggtggttaagtgtcgcccagttggtgctgagaattgagctcagttctgtagagagaacagaaaacgctctagcggctaagctgtgtctctcgggcgcttcactggccctcttttgactttttcttttttcttttttcttttctttttttttttttttctggagctgaggcccgaacccagggccttgggcttgctaggcaagcactctaccactgaactaaatccccaaccccttgactttttattttgagacatggtctcactaagtttcccaagctaactcccagctcaatctgtggcttaactggtgagcctgtagcacctctgccttggccacccaggttacatttctggggcaaaggtgcttccttttttttttcttcttcttcttcttctgttaaacagaagccaaggctgggctgactctccgaaccctcagggtcagaactggaaaacaggagaaccgagggtgtctctcagctacccacaggaagtcatcctcactggaaaacaatagcaagcacaaggcaggacacagaagtgctgactgaggtccggtcctctgtggcagagagccggtgcctcaggtcttcctagatgccagtgaggcccctctttcggggaagctcgttagcttcctcttcctaagaagttgtcagcttattttctgctgagaaccagagcagaaccaagattgtgagacccaccctagacagagggaacgcctctgccatgcacagcctttctggggtctcagatccccatggacttcattttgtctaggggtttattgctgttgattcagggtcttgtgtagcccaggctggccccaaacttcctatttcatcaaagtaaccttgaactcctgatcctctgacctccagtctggagtgctgagattacaggtgtacgccagcatgcatgaccaatgtcagtgtgccggggatcaaacctaggaacccaggacttcacaccggaaactgggccacatccccagccataatatctctctctctctctctgtctctctgtctctctgtctctctctctctctgtttcttttttctgagacagggtttctctgtatagccctggctgtcctggaactcactttgtagaccaggctggcctcgaactcagaaatccgcctgcctctgcctcccaagtgctgggattaaaggcgtgcgctaccaccgcccacttctttctttctttctttctttctttctttctctctctctctctctctctctctctctctctctctctctctctctctctctttcttcctttctttctttccttggtctcactatgtcactatgtagccttggctagcctggaactgctatgtagaccaggttgccctggtatccacagagatcaacttgcctctgctccctgaatgctgggattcaagatgtactacacaccccacccacaggtctcagtttttgcaagagcatccaactcatttggaactcatgattttccggcaacagttcctcaggtgctgggatcacttgtgtgggcatgcaccatcccacccagctaggacagtcatgtctctttgctctatatatacactaatgtgttcccagggccacgaataggtcacggcactagagagatgtgcactaagtctccgtttagaaacaggtagatggaggagttggggctcagctggtagaaagcttgcttaaatgggctccaccactggcactgcataaaccagtatggcggcacacacctgctgtctctcagcttttagcaagtggaagaaggaaaatcaggaattcaaagtcatctgtatctatataataagttcaagactggtctgggctgagagtgaaacaaacaaatgtgcgcgagaagaggcgctgtgagcgccgagtttgctgtgatgaagacctgggttctggtccccaggtccccttgcaaaggctgggcgtactgctgcgtgtctgtagctgcagggatgggtctggcagagacggagcctcaccagccactggcatggctgaaagggaggaccccaggttcatggagaggctctgtctcaaaaaataagtgagcccggcgtggtggcgcacgcctttaatcccagcacttgggaggcagacacaggcagatttctgagttcgaggccagcctggtctacaaagtgagttcctggacagccagggctacacagagaaaccctgtctcgaaaaaacaaacaaacaaacaaacaaaaccccaaaaaacaacaacaaaaaaaagtggatgtggggagagatggctcagtggttaggaacactccctgttcttgcagaggaccaggatttaattcctagctcccatagggtaactcaccaccatctgtaactccatttccaggggctctgatgtcctcatctggcctccacagacactgcatgcatgtggtgcagagacacacatgctaaaaagcacccagacacacagagtgaaaaatatataaaggggaaaaaatgcaaagagcgaaagaggaaggagcctgcaattgacctgtggccttgacccatgcacatgccacacacaccaaaataagggggtgcagtggtggggtcagcagggtggggtgtctgtcactctgggaagccctccaagtcagcacagtgctgatacagggaagtggagtctgtccatggcaagcttcacccaagccttggtgggggtggggtgggtgggtgaggtcatagcctagcctaactgctggcccatcccattgcctgtgtccctgcagagccgccgtctatgcgcctgaaggcccgtcctggcaactctggctcctccgtgctgacctgtgctgctttctccttctacccaccggagctcaagttccgattcctgcgcaatgggctagcctcaggctccgggaattgcagcactggtcccaa

SEQ ID NO: 5 - DNA, 1098 bp, Homo sapiens
atgggggtcccgcggcctcagccctgggcgctggggctcctgctctttctccttcctgggagcctgggcgcagaaagccacctctccctcctgtaccaccttaccgcggtgtcctcgcctgccccggggactcctgccttctgggtgtccggctggctgggcccgcagcagtacctgagctacaatagcctgcggggcgaggcggagccctgtggagcttgggtctgggaaaaccaggtgtcctggtattgggagaaagagaccacagatctgaggatcaaggagaagctctttctggaagctttcaaagctttggggggaaaaggtccctacactctgcagggcctgctgggctgtgaactgggccctgacaacacctcggtgcccaccgccaagttcgccctgaacggcgaggagttcatgaatttcgacctcaagcagggcacctggggtggggactggcccgaggccctggctatcagtcagcggtggcagcagcaggacaaggcggccaacaaggagctcaccttcctgctattctcctgcccgcaccgcctgcgggagcacctggagaggggccgcggaaacctggagtggaaggagcccccctccatgcgcctgaaggcccgacccagcagccctggcttttccgtgcttacctgcagcgccttctccttctaccctccggagctgcaacttcggttcctgcggaatgggctggccgctggcaccggccagggtgacttcggccccaacagtgacggatccttccacgcctcgtcgtcactaacagtcaaaagtggcgatgagcaccactactgctgcattgtgcagcacgcggggctggcgcagcccctcagggtggagctggaatctccagccaagtcctccgtgctcgtggtgggaatcgtcatcggtgtcttgctactcacggcagcggctgtaggaggagctctgttgtggagaaggatgaggagtgggctgccagccccttggatctcccttcgtggagacgacaccggggtcctcctgcccaccccaggggaggcccaggatgctgatttgaaggatgtaaatgtgattccagccaccgcctga

SEQ ID NO: 6 - DNA, 80 bp, Artificial Sequence
  connection sequence
agtcacgtgtcccttccactccgggtcaccctgtcggaatgggggtcccgcggcctcagccctgggcgctggggctcctg

SEQ ID NO: 7 - DNA, 81 bp, Artificial Sequence
  connection sequence
gctgatttgaaggatgtaaatgtgattccagccaccgcctgatgcagactcgggccccctgcccactgcagcctttcgggc

SEQ ID NO: 8 - DNA, 80 bp, Artificial Sequence
  connection sequence
agttgtgtatgtgatacctacactgtaccaggcattgcttagtactcgagctagagctcgctgatcagcctcgactgtgc

SEQ ID NO: 9 - DNA, 89 bp, Artificial Sequence
  connection sequence
aggcggaaagaaccagctggggctcgactagagcttgcggaacccttcgaattccgaagttcctattctctagaaagtataggaacttc

SEQ ID NO: 10 - DNA, 111 bp, Artificial Sequence
  connection sequence
gaagttcctattctctagaaagtataggaacttcatcagtcaggtacataattaggtggatcctagatctccacagccatgctcaaagcccacccagcatcagtacaaccc

SEQ ID NO: 11 - DNA, 1738 bp, Artificial Sequence
  the mRNA sequence of the transformed humanized mouse FcRn
aggagctagtgggtggagttggatgccctcagagttctccagtcctaactgtgtacagacaggatgtaagagaagaactggaggctctaagcagaggatccatcggctgcaggcagagggaagagggcctctgtgaggaacaggctgagcgtcagaggaggaggcccaggcctggttctctagctctgtaattaattaactaaagtggatcaaatgagaaggtgaaagttcacagaggaacactcctgtctgtcgtcttggactgggtctccatcccaccatccagcgtcctggtctacgaagagtccacagggaccttgtgaagaatcaacaaggcggggtccagaggagtcacgtgtcccttccactccgggtcaccctgtcggaatgggggtcccgcggcctcagccctgggcgctggggctcctgctctttctccttcctgggagcctgggcgcagaaagccacctctccctcctgtaccaccttaccgcggtgtcctcgcctgccccggggactcctgccttctgggtgtccggctggctgggcccgcagcagtacctgagctacaatagcctgcggggcgaggcggagccctgtggagcttgggtctgggaaaaccaggtgtcctggtattgggagaaagagaccacagatctgaggatcaaggagaagctctttctggaagctttcaaagctttggggggaaaaggtccctacactctgcagggcctgctgggctgtgaactgggccctgacaacacctcggtgcccaccgccaagttcgccctgaacggcgaggagttcatgaatttcgacctcaagcagggcacctggggtggggactggcccgaggccctggctatcagtcagcggtggcagcagcaggacaaggcggccaacaaggagctcaccttcctgctattctcctgcccgcaccgcctgcgggagcacctggagaggggccgcggaaacctggagtggaaggagcccccctccatgcgcctgaaggcccgacccagcagccctggcttttccgtgcttacctgcagcgccttctccttctaccctccggagctgcaacttcggttcctgcggaatgggctggccgctggcaccggccagggtgacttcggccccaacagtgacggatccttccacgcctcgtcgtcactaacagtcaaaagtggcgatgagcaccactactgctgcattgtgcagcacgcggggctggcgcagcccctcagggtggagctggaatctccagccaagtcctccgtgctcgtggtgggaatcgtcatcggtgtcttgctactcacggcagcggctgtaggaggagctctgttgtggagaaggatgaggagtgggctgccagccccttggatctcccttcgtggagacgacaccggggtcctcctgcccaccccaggggaggcccaggatgctgatttgaaggatgtaaatgtgattccagccaccgcctgatgcagactcgggccccctgcccactgcagcctttcgggctgtgtgacctcctgaactgtctccgagcctcctgagggagcctgggcccgatgtcctcccatggatccctgcttttgtggcctgcttcagtttcccttcttaatgtacatggttgttttccatctccacataaatttggccccaaatctgtgtgtgcatcgttattctcaagtttcaagcagctggaataaattgaacgcgtctgggaaagatc

SEQ ID NO: 12 - DNA, 22 bp, Artificial Sequence
  primer
tgtcaagtacgaatgtgtatgc

SEQ ID NO: 13 - DNA, 20 bp, Artificial Sequence
  primer
atgacgattcccaccacgag

SEQ ID NO: 14 - DNA, 20 bp, Artificial Sequence
  probe
aggcaggcagatttctgaca

SEQ ID NO: 15 - DNA, 21 bp, Artificial Sequence
  probe
tccagaagcctgagtttagct

SEQ ID NO: 16 - DNA, 25 bp, Artificial Sequence
  probe
cggttcttatattcagtgaggaaac

SEQ ID NO: 17 - DNA, 20 bp, Artificial Sequence
  probe
ccaatcctgcacacagctac

SEQ ID NO: 18 - DNA, 20 bp, Artificial Sequence
  primer
ccagcgtcctggtctacgaa

SEQ ID NO: 19 - DNA, 20 bp, Artificial Sequence
  primer
ggtggcctcttacctgagcc

SEQ ID NO: 20 - DNA, 23 bp, Artificial Sequence
  primer
ggctattgtagctcaggtactgc

SEQ ID NO: 21 - DNA, 3790 bp, Artificial Sequence
  5i - homology arm
caagccagggcatccccatagtcccagcactcaggagggagaaggttaaggttagtgtcttctgcctctctctttttctaccccctcccctttccccctctcccctccctccatctttgtatgggttccaggggtcaaactcagatcatcaggccttctgtcgcagatgcctttagctgctgagccgtctccatggtccccggaaggggttcaaacacaaattggtgcacagggaggttcatagaaaaagaggcattaagggggcgggcccaagcagggacctgggcttctcaaaggggagtctcacgtaagtgtcttaactgtaaccatacacaggggttgggaggctgccttccccacctcgttcttagttaagtgaagtcagagtgcttcctggagggctctggggaggatggcagctgaagaggtaaactggagactgtagggttgcacaagggaagaaagccagcttcttgccgtggacaaaggttccggactgatttgtaagtttggggtttccatatgggaagaaaaaggttattggtgacagccattgagtggagctgattgctgtggtgttagtattcttgtttgaaatctctctccaccatcacaaaggccacagcaatccctgctaactttttttttttttttttttttttttttttttggtttttcaagacagggtttcctcgaactcagaaatctgcctgcctctacctccgaagtgctgggattaaaggcgtgtgccaccatgcccagcccctgctaacttcattgtggcttgactctcagctgtccgcagactttagactctggtgtgacaggctttccatgaacacttccctgccaatccttctttcttcttccctccttccctccttctctcctcccttcctcccttccttctttccttcttccctccttcctttgacagggtcctttgtcgcccagcctggttttaaactttctatgtaattgaggatgaccttgaactctgttgtcctgcgtgctagacaagtagatttacttcattaaatgaaccaaagcccccagacctcatatggtttttgctgttgtggaaacctgactcccgctcactgtgtagatcaggctagtcttgagcacacagggatctgcctacctctgtgtcctgagtgctaaggttaaaggcgtgagcggccactcctgttctttttcagttttacttcctggatttgcttggttctctgtgtccctgcgtgttgagtacagtgccctctgaagccggtagctcccccgtagctggagttacagatggttgtgagcatcgtgtggatgctggcagccaaactcaggtcttctacaagagcagccagtgtttttttttgttgttgttgttgtttggtttttcaagacagagtttctctacgtgaccctggctgtcctggaactctctctatagaccaggctggcctcgaactcagagattcacctgcctctgcctcctgagtgctgggactaaaggtgtgagccaccaccagcacagacagtgtctcttaatcacctagccatctctctagccacagacgcagctttttacaggggaggctagcatcagaagatcaggactcaggtcctcattcttggcaggcaaacacttactgactgagccctctccccagtccgtaggaggcctgcaagcccagcacttaagagctggaggctgggcttcaggagttcatggtcgacctgggctgcatgagttcctgtcctgacacatctaccctacccctctgctgcctccacgctaccaccaccaccaccattattacacacgcacacacttgtacacacacttgatatagaccagagggagagggaggtcaaggcaggtaggagtggttgactttctattctctcttaaaaaggttttttttttaatttatttattggttatatgtaagtacactgtagctgtcttcagacactccagaagagggaatcagatctcgttacggatggttgtgagccaccatgtggttgctgggatttgaactccagaccttcagaagagcagtcgggtgctcttacccactgagccatctcaccagcccttaaaaaggttttttagatttatttattttattcatgcctgtgtgtatacacgtgccccacatccctgcctggtgttcaatgagaaattgtcagaccccctagaactggagttatgggcagctgtgagccaccatgtgggtgccgggaaccaaactcggatcctctgcaagagcagcaggtgctcttaactgctgagctgtctcgtcagctccagactttccagactcttgcttctgtctttcaagtgtgtgggttccaggcctgccccatcacaccggttttgcccatctacttcagcctgttggtccatctgctgaccaccagtcgaccactcacggctctgtcagtctccctctctcggctgtttatattccctgccgcccaggccggccatgaacccctggcctcccggcctccatccttagtgctggaattacaggcatgtgctgccgcccaccccctttcttctttgctgtctatctaccctgtcaacctacctgtctgtcagtgtctctctgtgtgtctgtgtgttttttctggcaagatcttggggtgggcagcagggtgggagacaggggccaaaggcaaaggctctcatcacagagtgtgtaggcagggttggggccacctcaagtgctggcacatggaagaaatcagagcaggcttacagctgggagtgggctgctgccaaaccttgtgtgttaggcaaccatgccatgaccaagccacacccacagcccacaaatcctggggtgtcttaaagtcacattcagcgtgcaggcctttccagggctgcgagtctactccaaaaatgctccaaacgagtaaacatagaagctgttttgggaggcaggcctaatgacgtttgccccgggaggcttccaaccagatacagcaggaacttcaagagaggctggacagaggcccaaggggtgtgcttaggagctagtgggtggagttggatgccctcagagttctccagtcctaactgtgtacagacaggatgtaagagaagaactggaggctctaagcagaggatccatcggctgcaggcagagggaagagggcctctgtgaggaacaggctgagcgtcagaggaggaggcccaggcctggttctctaggtatgagggaccagcaccccaagaggaaagagggttggggcctggagctccaggtctgagggaggagtgttatgacctgagggggagagaggaggctggggtctgaggaaggagagcaggaccccgggcacctgagtctgagggatgaagccggagcctagggttttgggtttgaggaaagatcttggaactggactcctggatctgagggaggtggaactggactcctggcttgggtttgagggctcctgaggctgaaatctgagcccctgggaatgagggaggaggagaaggcctggatcctctggtctgagggaggagaaaaaggaaataacagaagtctgagacaaattttggtctgtctagctctgtaattaattaactaaagtggatcaaatgagaaggtgaaagttcacagaggaacactcctgtctgtcgtcttggactgggtctccatcccaccatccagcgtcctggtctacgaagagtccacagggaccttgtgaagaatcaacaaggcggggtccagaggagtcacgtgtcccttccactccgggtcaccctgtcgga

SEQ ID NO: 22 - DNA, 4540 bp, Artificial Sequence
  3 homology arm
gctgggacttgaactctggtcctctggaagaacagtcagtgctcttaattgcagagccatctctccagccgcgctccccccccaccccccccttttttttttaaagacaagggtctctctacgtcgtcctggctggcggcaaactcatagagacagactgcttctgtctcccaagtgagccaaccaaacccgacctcctcttttctgaaacggatctaagaaatggaaagagtttttaaaaaatacattccccttaatcccctggggccagagactctggaggtggtcgtgcaagtcggcccatatgtttgctgggagttgtagttcaatatcagccttatcgcccaccacgccttcgggtctggagctcggccttgtggacgtggcaagggggtcgtcattggctccgcccctgaggctttcctgcgcagtcattggtcagagggcggcagcctctggacggctggtggaggagcttaactggaacctggccaggttgggcatctttgtgcgaaagcagagctcagcgtagggagctaactcaggccgggtaacagaggtgtggtggggtgatggtggggtgaaggtggggtgggtggaagggtgggggtcgcggtccagcctgggggcagcacagaggagcctgcatgatgctttcggccaccaccatgccctgaactttgggtgggacatgcgccacgtctctaactgaaataccccctttctgctctgagactcctctgaggctcggattccacccccagaggttgttttgcctttaaattctctattctttttgccgacttttcttgagcccaaaactttttgcagcctgcgcttttatcggccctgccttaggcagcctcctctactccgcccccttcaaagttctcccacattatctctggttcagacaccccccgcccccagtcttatccctgaccgctggtcctcacctctcctcaggaaccgatgatgtggcgatggtcgtttctgttgcttctcttactgctgaggcactgggccctcgggaagccatcccctgatgcaggacctcatggccaggatagggtccatcatggaacccccctgagcgaggccccccacgatgacgcccacgggaatttccagtacgaccatgaggcgttcctgggacgagacgtggccaaggaatttgacaaactcagcccagaggaaagccaggcccgacttgggtaggaggatggcgggggagcggggcagaagtgggaggtggggagagagaagttaactgaaaagacaaaactgtggcactcacctcctgggagaaggcggagctaaacctgaggctgtgggggacctgcgtcaccaacccctgggttcccctctacctcagtcccccctatgcctctgtctggacttccctcttctctcttctaaacgggacgccgactctcggtagagactgaagacaagggatttgaggaagatgccaaggtcagagatcaggttgacctgggatgagtgatgccaactggactggacgggcttgccccgtttctaggtgacagatacagtcagcatgggcccgggacaagaaggccactcgtgcaaatatgcttgattattttcattgttattagtactttgagatcgaatttcatttgtggaactagctagtctgaaattcactctgtagacgagactggtcctgaattcagaaatccgtctgcctctgcctcccgagtgctgagattaaagatctgcgtctttaatgtgatggcaatttctaattttaaactgggcaaatagtagcagggcagagctggagagagaggagccccaaattgatcagcccatctagggaagtgggcagactgcacttgaaagaggggcgggccctgagatggtgagaggcggagcttcaaggttaatagagacagtggcactctaggattggggtaaagttagaggccagacaccctgttcacacttaagctcaagtccagctagggtctgtgcagcgcttagaggtcggggctagagtagagggtcttgatttgtgtcaggtgctaatccaactaactcctctaggactcctgtaggggacaggctaggcttttagatgggcctggtactaagattaatagatgcagctccctcagttcacctagcatttctactctttcctagcctgtgcacccatgtctctgaggggcgtggctagggcaaggggcggagtcagggttggcaaagacctaggggtgggtgaaacctatcaacggtaaagcttctcctggtcctgcactggctcacagttttaatcccaggacttggaaggcagaggcaggcggatctccgtgagttcaggaccagtctggtctacagagtaagtttcaagctaaccaggagtacataatgataccccctctctcaaacaaaaccccccaataatacgttgaaatctaccaaacagaaaacagacagttcctctcctgggttcatttggagtcccaccccagtttgcttgcagaccttgggtgacatttttctcccttatctttctttctttctttttaagatttattattatatgtaagtacactgtaagctgtcttcagaccagaaaagggcgtcagatctcattactgatggttgtgagccaccatgtggttgctgggttttgaactcaggacccctggaagagcagtcagtgctcttaacctttgagccatctctccagccctctatctttatttctctctctctctctctctctctctctctctctctctctctctttttcttttttggttttttgagacagggtttctctgtgtagccctggctgtcctggaattcacgctgtagaccaggctggcctcgaactcagaaatctgcctgcctctgcctcccaagtgctgggattaaaggcgtgcaccaccatgcccagctctttttttgtttttgtttttctatctttatttctaaataattgtttcattttatttaagtatatggatatttggcctgcatgacatgtgtgtgcaccacacgcatgcctggtgccatggatcccctggaactggagttacagatagtcatgggtgctggagttgaacctgggtaccctaaaggagcacccagcgcttctaactacagagctgtttcttcagcttccttatttttcttttattattttttttcttttttgagacagtttatctaaatagtccttgctgtcttgtagactaggttaatcttgaactcacagagatctgcctgcctatacatcccaagtgctgagattaaaggtctacacaactatgctctgctctttattattattattattattattattattattattattattatcatcatcatcatctaattaaaaaagatttatttatatgagtgtgggtattttttttctttttctaaagatttattattatatctaagtacactgtagctgtcttcagacacaccagaagagggtgtcagatctcattgcagatggttgtgaaccaccatgtggaaattgaaggaactcaggacccctggaagagtagttagtgctcttaaccactgagccctatctccagccttctttttctttttttgtgtgggtatttttcatgtgtgtccattaaccattaacatgtcttgtgcccacaagggccaaaagaggataatgggttcctgcatctagagttagagatgcttgtgacctgcctcgtggggctcatgggaatcaaacccaggttcctacaagagcagccagggttcttaaccgctgagccatcaccaagtccccatttttgtcttatataggtacaagggtaactctatatatgacagggaggggcctaggtgggcggggtcagggttgagattgactgcgtctctaactgtaggggcagggcctcattgggcagagccagagactgacattcattcccctcgcaggcggatcgtagaccgcatggatcttgctggggacagcgacggctgggtgtccttagctgagctccgcgcgtggatcgcgcacacacagcagcggcacatccgtgactcggtgagcgcagcctggcacacctacgacacggaccgcgacgggcgcgtgggttgggaggagttgcgcaatgccacctatggccattatgagcccggtatgcccacctcgcctttgccctttgacctcgcttttaggatcttatccctgataccttccctccatttcaaatgtcatttgctcttccttctgaaatccaatctatgtgacaagttgactggagacttcacattcgtggcaggaatacttaatgtttaaccctgttatccagacccatatgtggcactggtgtcttagctagggttaatattgctatgatgagacactgtgaccaaaagcaagtgggagaggaaagggtttgcttggcttacgcttctacgtctgcggtccatcactgaagaattccctatagcactggttccccaccagtgggtcacaacccctttgtgagatactgtgtcagagatactgtgtttcaga

SEQ ID NO: 23 - DNA, 12719 bp, Homo sapiens
atgggggtcccgcggcctcagccctgggcgctggggctcctgctctttctccttcctgggagcctgggcgcaggtgagggccgctccgggccagggccctgctgcaggcgggcggcgggaggcggccccaggcaggcaggggcgaagccagcgggacccgagttccccgcgagcccctggcgctgccttctccgctcaattaactttctcaacgtctcctgctgggcctgaggctgggaaccacctgtcgcctcttgtgtctgtttccctctctctctctgggtctctgtccccctctctttctgggtctcttgtctctctctctctgggtctctgtccccccccccccgggtttctgtcccctctctctgaatctgtccccctccctccataatagattcttctccctccctgggtatctgtcccactgcagtctagttccccgcccgtgtgctcccttcagctctgtttctgtctgcagaaagccacctctccctcctgtaccaccttaccgcggtgtcctcgcctgccccggggactcctgccttctgggtgtccggctggctgggcccgcagcagtacctgagctacaatagcctgcggggcgaggcggagccctgtggagcttgggtctgggaaaaccaggtgtcctggtattgggagaaagagaccacagatctgaggatcaaggagaagctctttctggaagctttcaaagctttggggggaaaaggtgagattccggtctggaggggcaaggggccgggtccatgctccggggccccgcttacctgtgtttgggcgccccaggtccctacactctgcagggcctgctgggctgtgaactgggccctgacaacacctcggtgcccaccgccaagttcgccctgaacggcgaggagttcatgaatttcgacctcaagcagggcacctggggtggggactggcccgaggccctggctatcagtcagcggtggcagcagcaggacaaggcggccaacaaggagctcaccttcctgctattctcctgcccgcaccgcctgcgggagcacctggagaggggccgcggaaacctggagtggaagggtgagccggatctgcagccgcaggctgttctgtcctctctcccgtcatgcccacctgcctcagttcccctgccaggaccctccatcagcctcccactgcagcccacgctctgcccccccattcctcaggggtccttctacactcagccctcccatggctccccagcgccccccaggacaaactcctggcttccttccttgccttctttttttttttttttttgtgagacggagtttcgctcttgttgcccaggctggagtgtaatggcgcgatctcggctcaccacaacctccgcctcccgggttcaagtgatctcctgcctcagcctcccgagtggctgggattacaggcatgcaccaccatgcccggctaattttgtatttttagtagagacggggtttcttcatgttggtcaggctggtctcaaacccctgacctcaggtgatccgcccacctcagcctcccaaagtgctgggattacaggcatgagctactgctcctggctccccccttttttttttttttgagacagattctcgctctgtcaccccaggctggaatgtggtggcaccatctcggctcactgcagcctgaacctcccaggctcaagcagtcctcccacctcagccaaccaagtagctgggaccacagcccagcatcatcacacctggctaactttatattttgtagacacggtgtctcactatgttgcccaggctggtcttgaactcctaggctccagcgattctcctgctttggtctcctgagtagctgagattacaggtgcataccaccataactggctaatttttttttttccgcacaatctctgttgcccaggctagtgtgaagcagggtgatctcagctcgctgcaacctccgcctcctgggtttaagggattccgctgcttcagcctcatgagtagctggcactataggcacatccaccatgtctggctaatttttatacttttagtagagatggggtttcgtcatggtagctaggctggtctcaaactcttgacctcaagtgatccacccacctccgcctcccaaagtgctgggattacaggagtgagctaccgcgtctggccatacctggctaatttttaaaatttgttgtagaggtggcatctcactatgctgcccaggccagactcaaactcctggcctcaagtgattctctcactttggcctcccagggtgctgggatgacaggtgtgagccactgtgcttggccttccttgccttaatgtcctcatcagacccacgcagcagcacttgtctctacgcagccatcagagtctacgctccagccacccaacataagcaatctcctccctctgggcatttgctcatccagtttcccctggcacctctctgttccacccttccccctctctgcatatactccagccctccttattcttcaaggtcagggctggatatcacttcctctgagaagcctttcctcagctctgtagggcagagttcactgtttcctccccggaggcccccagatccccactcattcaactgttggccaagagcatttgcactgccaggcgctgctctgggccttgagcaaagagggaagtgacacacattcaccctctgtggctcccaggctgctggacaaagatagataccagctggggatgtgcacagatattcattccaagtgccacaaaggagaagttgagactcaaccaggtagtttttctttaattgtcttctttctttcctaagtagggaaaatttctttctttttttgagacagagtttcgctcttgtcgcccaggctggagtgcaatggcgcgatctcggctcactgcaacctccgcctcccaggttcaagtgattctcctgcctcagcctcccgagtagctgggactacaggcgtgcaccaccacgcctggctaatttttgtttgtttttttttttttagtaaagacagggttttaccatgttggccgggatggtctcgatcttttgacctcgtgatctgcctgcctcggcctcccaaagtgctggaattacagacgtgagccactgctcctggcctttgttgttgttgttgttgtttttgagacagactcttgcactgtcgcccagactgcagtactgtggcgcaatcttggctcactgcaacctccgcctcccaggttcaagcaattctcctacctcagcctcccgagtagctgggattacaggcacacaccaccatgcctggctaatttttgtatttttagtagagacgggggcttcaccatgctggccaggctggtctcgaactcctgatctcgtgatctgcctaccttggcctcccaaagtgctgggattacaggcatgagccaccactcctggcttaaggagggaaagtttcaaaccacacttaagtagaaacaatagtataaaacggaccagacacggtggctcacgcctataatcccagaactttgggaggccgaggcaggtggattgcttgatcccaggagttcaagaccagcctgggcaacatagtaagaccgcatctctactaaaaactcaaaaaattagcctggcatggtggtatgtgcctctagtcccagccactggggaggctatggcaggaggatcgcttgagctcaggagtttgaggctgtagtgaactgattgcaactctgcactccagcccagatgacagagccagaccctgtctcaaaaagaaaaaaaatatggtggccaggtgcagtggctcacgcctgtaattccaacactttgggaggccgaggtgggcagatcacttaaggtcaggagtttgagataagcctggccaacagagtgaaaccccatctctactaaacatacaaaaattagccgggtgcggtggtgggtgcttgtaattccagctattcgggaggctgaggcaggagaatcacttgatcccgggaagcagaggttgcagtgagccgagatcaagccactgcactccagcctgggcaacaaagcaagactctgtctcaaagaaagaaggaaagaaagagagaggagagggagagagaaagaggaaggaaaggaaggaaggaaggaaggaagcgagcatcccatcactgagacttaataattatcaacccatggttttttgaatttcacgcacatcctcactcacttccccaccccacttcacatcaagctttggtaaatctcagacatcacagtgtttttttctttgtttgtttgtattttgagatggagtctcactctgtcatgcgggagtgtagtggctcagtctcggctcactgcaagcttcgcctcccaggttcaaacaattctcctgcctcagcctcctgagtagctgggattacaggtgcccgccacctcgcccagccaatttttgtatttttagtagagatgaggtttcaccatattggccaggctggtcttgaactcctgacctcgtgatccacccaccttggcctcccaaagtgctgggattacaggcataagccaccgcgcctggccaatttttgtatttttagtagagacgggatttcactatgttggccaggctggtctcgaaccctccacctcaagtgatctgccctcctcagcctcccaaagtgcgggagtcaccgtgcccagccagactgttttgttgataagtcctttagtatgtaactctactaggtaagggcatttaaaaaaaattaataccaaaaccatcatcacacctacaaccaattaactctttaatggcatcagatgtccagtttttgttcccatattcctgactgtaaatgttttttaaagtttgaatcagcatccagcaaggtccacacattttgttgtcttttcagtgtctttttctgattgttgaatttttctttctttctttttttttttttttttcctttttgagacagggtcttgctctgtcaccagactggagtccagtggtgcaatcatagctcactgcagtctgaacttcctgggctcaagtaattctcccacttcagcctccccagtaggtaggaccacaggtgcacgccatcaggcctagctatttttgtttgtttgttttttgagacaaagtcttactctgtcgcccagactggagtgcagaggcacgaactcggctcactgcaacttctgcctcccgggttcaagcaattctcctgcctcagcgtcccaagtagctgggattacaggcgcaagccaccacgccctgctaattttttgtatctttagtagagacagagtttcaccatgttggccaggctggtttcgaactcccgaccccaggtgatccacccacctcggcctcccaaagtgctgggattacaggcgtgagccaccgcgctcggccgtatttttgtttttttcttttttttggagacggagtctcgctctgtcacccaggctggggtgcagtggcgcgatctcagctcattgcaacctccacctcccaagttgcaccaattctcctgcctcagcctcccgagtggctgggactacaggcgcccaccaccacgcccggctaattttttgtatttttagtagagacagagtttcactgtgttaggcaggatggtctcgatctcctgacctcctgatccgcctgcctcagcctcccaaagtactgggattacaggcttgagccaccgcgcccggccggattccaattttttaccagataggtgttttgcaaatatttcttccactctctaggttgtcttttcattgttttaactgtgcctttcacaaagcaaaagtttttaactttgatgaaatccagtttattcttttttttttttctatcgtggattatgcttttggtgtttaatctcagaagtaatcaccaaacccaaagtcagctagatcttctcctgtgtattttaagagttttaaagttttgcattttacagttaggtctgtgattcattttcaggtaatttgagttacttgtgatcttgagtgtcttttgatttacaggtttcccattttctttttgttttttttccccctgtaattgtggtttgaagaaagtcagtttatgtcctattgagtttctcctagactggattttgccctttgcatcctgtgatgttgctggaaatagtcctccttctcttctttaaaggagcagctccatctagaagcttggtcagattctggttcaatttttatggcaagaaaactttctgggtagttggtgcgtatgtctgtgtgcataggctgcaaacatctgcttgcttctgtgctgttctgtggaccactggattcgggtgttgttatgaagttctttgtcaggttttctcctgatggttttagcggatagcaatggccattgccttaatcttttttttttttttttttttttgagatggagtcttgctctgtcgcccaggctggagtgcagtgttgcgatcttggctcactgcaacctccgcctcccaggttcaagcgattctgctgcctcagcctcctgagtagctgggattacaggtgcgtgccaccatgcctggctaatttttgtattttttttggagagatggggtttcactatgttgggcaggctggtcttgaactcctgaccttgtgatccacttgcctcggcctcccaaagtgctgggattacaggcgtgagccaccgcgcccgggcttttttttttttttttttgagagggcgtctccctctgtcaccaggctggagtgcagtagcacgatctcggctcactgcaacctccacctcccaggttcaagtgatcctcctgcctcagcctccttagtagctggaattacaggcacacaccaccacacccagctaatttttgtatttttagtacagacagggtttcaccatgttggccaggatggattttttttttttttttgagatggagtttcactcttattgcgcaggctggagtacaatggcatgatctcagctcactgcaacctccgcctcctggattcaagtgatcctcctgcctcagcctccagggtagctgggactacaggcatgcgctgccatgcctagctaatttttatatgtttagtagagatggagtttcaccatgttggcgaagctggtcttgaactcctgacatcaggtgatctgcctgcctcagcctcccaaagtgctggggttacagacgtgagccaccatgcctgacctggtttggtattttgttaaacagaaccaaaggccagacagattttctgtgcttggatttagtctatattggaagtaccttgaggctgaaggcacgtggctgtgagcacgacacaggaagatacagatttgctgaaaagaatagtgaacataaagcagaagccagaactgctgatggccgtcagcccggtccctccattccacctgacggtgcagcaaattctgccgacttgccagagaggcagctccaggagggagaagctgctgtgctttcctgaagcagcaaatgtgttttctgctgagcaaccaaaaccaagatcccagccaagacccacgattgcccagatccaccctccctgaaaggaacttgggcagtaatgcccagtcctaggcagttgctgaaatctttgccttttattttcttttttgagctgggatcttgctctgtcgcccagactggagtggagtgcagtggcacagtcatagcgccatgtagccttggactcccaagctcaagagatcctcccgcctcagccttacaagtagctgggactataggtacacgtcaccatgcctggctaacattttctattttttgtagagatggggtatttgttgccaaggctggtctagaagtcctgggcttggccgggcacagtgactcacgcctgtaatcccagcactttgggaggctgaggcaggcggatcacgaggtcaggagttcaagaccagcctggccaatatggtgaaaccctgtttctactaaaaatagaaaaattagctgggcgtggtggtgtgcgcctgtaatcccagctactctggaggctgaggcaggagaatcgcttgaccccaggaggcggaggttgcagtgacccaagattgtgccactgtactccagcctgggcgacagagactccgtctcaaaaaaaaaaaaaaaaaaaggaaaagaaagcctgggcgtaaggatccttctgccttgacctcccaagtgctggaattgcaggcatgagccattgcacccggcttttttttttttttttctttgagacaggcacttgcactgttgcctagactggagtgcagtagcgggattagagttcactgcagcctcaacctcccaagctcaagcgatcctcccaccttacccttcctgaatggatggggccacaggtgtgtaccatcacacgtagttaatttttaaactttttcagagacggggtttctccatgttgcccaggctggtcttgaactcccggcttccagtgatcctcctgcctcagcttcccaaagtgctgggattacaggcatgaaccactgcacccaaccttttttcattcccttaacgaaacctttggatgaacaaaaattcttaatttcagtgaagttcagtttatcgacttcaatttatacattttataaagtttatgaatttttttttttttttttagacggagtcttgctctgtcgcccaggctggagtgcagtggctatacccaggtgtgatcataactcactccagcctcaaactcctgggctcaaggattcctcctgcctcagtctccagagtaggtgggaccacaggcacatccaccacactggctaattttttttatttgtagtggagactacgtcttactgtttgcccaggctggtctccaactcctgggttcaagtgatccacctcattctcccaaagcgctgggattgtaggagtgagccactgcacccagcctgtctgctaatattatgtttaagagttctgaatctttttttaaattttttaaaatttttaattttttattttttttgagacagtcttgctctgtcgcccaggctggagtgcaatggtgcaatcttggctcaatgcaacctccgccttctgggttcaagcgattctcctgccttagcctcctgagtagctgggattacaggtacacgccaccatgcctggctaatttttttgtatttttagtagagatgggatttcaccatgttggtcaggctggtctcgacctcctgacctcgtgatcctcctgcctccaccccccaaagcgccaggattctaggtgtgagccaccacacctgtcctgaatctatatttatgagagacattggccttaagctctctttttttttttttttttttttttttgagatggagtctcgctctgtcacccaggctggagtgcagtggcgcaatctcggctcactgcaagctccgcctcccgggttcacgccattctcctgcctcagcctcccaagtggctgggactacaggcgccggccaccacgcccggctaattttttgtatttttagtagagatggggtttcaccgtgttagccgggatagtctcaatctcctgacctcgtgatccgcctgcctcggcctcccaaagtgctgggattacaggcgtgagccaccgcgcctggccaagttctctttcttataatgtgctttgttgtcaagatgatgctgacctcataaaatatgctgggagagtgtttcctctgttttatggcctgaagagttagtgtaggaatagaatcatttctacattggaagaattcactaattgatgtcttctggacctggaattttagtttttggaaagttttaaactatggattaaaaattttaaataggccgggtgtggtgcatcatgcctgtaatcccagcactttgggaggctgaggcaggtagatcacaatgtcaggaatttgagaccagcctggccaatatggtgaaacaccgtctctactaaaaataaaaaaattacccggcatggtggtgcatgcacctgtagtcccagccactcgggaggctgtggaagaagaaactcttgaacccaggaagtgggggttgcagtgagtcgagatcgtgccactgcactccagcctgggtgacagagcgagactcttatctcaaaaacaaaaggctaggcgtggtggctcacacctgtaatcccaacactttgggaggcccaggcggatggatcatgaggtcaggagattgagaccatcctggctaagacggtgaaaccccgtctctactaaaaatacaaaaaattagccaagtgtggtggtgggcacctatagtcccagctactcaggaggctgaggcaggagaatggcgtgaacccaggaggcggagcttgcagtgagccgagatcgcgccactgcactccagcctgggagacagagcaagattccatctcaaaaaaaaaaaaaaaatttaaatagttataggactacagacgttttaaatttcctcttgtgtcagtttagaaggttgtatttatcaggaatgtgttcagttcatctaaattttcaaatgtgttgctgtggaatttttcataattatcctctatctttttttttttttctttttttctgagacggggtctcactctgtcgcccaggctggagtgcagtggcacgatcctggctcactgcaacatctgcctcccaggttcaagcaattctcctgcctcagcctcctgagtagctgggactacaggtgcccggctaatttttgtatttttagtagagatggggtttcaccatgttggccagtgtggtctcgaactcctgacctcaggtgatccacccactttggcctcccaaagtgttgcgattacaggcgtgagccaacatgcccagcccaaggaggtgacatcttgaggggacaccttcggggtggacaggttgagtctctgtcacctaggaagtgcctccccttttaccactgtattatctgcggttagcacagtgcaaacatggaggcctctttctgtccctatccagccctggctctgggagtggtgggctcgcgacttaggcctgtctgcctgatttcctgcagagcccccctccatgcgcctgaaggcccgacccagcagccctggcttttccgtgcttacctgcagcgccttctccttctaccctccggagctgcaacttcggttcctgcggaatgggctggccgctggcaccggccagggtgacttcggccccaacagtgacggatccttccacgcctcgtcgtcactaacagtcaaaagtggcgatgagcaccactactgctgcattgtgcagcacgcggggctggcgcagcccctcagggtggagctgggtgaggtcccgccaggtggtgatgctcctggtttcccgttgccttgtctcactgctgcgccggtccttcctgagtctgaccttcctccccactgctgccacctccttgaatctgactgccttgaacctcacgcctgtcagtgcccccaaaacctgatggcttgtccttcccaaggccaactgccttccgtctcctgctgcttctggcctcactgagtctgaagagctgttaactaccatggccagtcctccctgagtctgaccatcttccatcctgctgctgctgctgctgctgctgcgggtcttcctggaatctgaccattcgttgtctgctatgcccgtcctcaccaagactgactgcctgctgctttgctactgcccgggcccatgagactgacttcccactgctctgcctgcctctccccactgcactggcacagccccgccttgccgctgctgatccattgccggtgtgaccgcggccgctcgtgctgtagctggttcttacgtccaacctgggggcagtctgcccagatcgcctgcctggcctcgcctctgcccatcagacacttggtgctggaatctccgaggctgggagggactgggagcgccccagttctgtgtcctgactgggtggggtggaggggctgctccacatctcacagcgttctctggctgcagaatctccagccaagtcctccgtgctcgtggtgggaatcgtcatcggtgtcttgctactcacggcagcggctgtaggaggagctctgttgtggagaaggatgaggagtgggctgccaggtggggggcagcgggaggaagagcctctgagagagggacagagaccccaagagaggggcacacagacctgggaggacagagacccagagaggggggacagagatcagagagaggtggagacagaaacccagagatgggagaacagaggtgcagagagagggggacggagacagagacccagagacgggggaacagagacccagaggggacagagagaggtgcggacagagggagggggagacagacccagaggagggaggcaaagatgtagaaagaggggggacggaaaattgggagaaggggagacaaaggcccagaaggaaggggatagagactgaggaagagtgtgtgagacacacacacaaatgggggacgccagtcagacccagagcgcctcagagattctgatgacctctccctctctctctctcctcagccccttggatctcccttcgtggagacgacaccggggtcctcctgcccaccccaggggaggcccaggatgctgatttgaaggatgtaaatgtgattccagccaccgcctga

SEQ ID NO: 24 - DNA, 80 bp, Artificial Sequence; identical to SEQ ID NO: 6
  connection sequence

SEQ ID NO: 25 - DNA, 81 bp, Artificial Sequence; identical to SEQ ID NO: 7
  connection sequence

SEQ ID NO: 26 - DNA, 105 bp, Artificial Sequence
  connection sequence
attggattccattacagatggttagccaccatgtggttgtcgacggtatcgataagcttgatatcgaattccgaagttcctattctctagaaagtataggaactt

SEQ ID NO: 27 - DNA, 96 bp, Artificial Sequence
  connection sequence
aagttcctattctctagaaagtataggaacttcatcagtcaggtacataatggtggatccgctgggacttgaactctggtcctctggaagaacagt

SEQ ID NO: 28 - DNA, 23 bp, Artificial Sequence
  primer
cagatagagcaagccgtggagag

SEQ ID NO: 29 - DNA, 24 bp, Artificial Sequence
  primer
ctcccaggaaggagaaagagcagg

SEQ ID NO: 30 - DNA, 20 bp, Artificial Sequence
  primer
aactgttcgccaggctcaag

SEQ ID NO: 31 - DNA, 24 bp, Artificial Sequence
  primer
cacctctcaagtgctgagactgag

SEQ ID NO: 32 - DNA, 25 bp, Artificial Sequence
  probe
aacctggagctcactggtcagcaaa

SEQ ID NO: 33 - DNA, 24 bp, Artificial Sequence
  probe
cacgggcagactctcagttccatc

SEQ ID NO: 34 - DNA, 25 bp, Artificial Sequence
  probe
atagcccagagctcaaggataggca

SEQ ID NO: 35 - DNA, 25 bp, Artificial Sequence
  probe
cccagtctgctttctgtcctgtcac

SEQ ID NO: 36 - DNA, 21 bp, Artificial Sequence
  probe
ggatcggccattgaacaagat

SEQ ID NO: 37 - DNA, 22 bp, Artificial Sequence
  probe
cagaagaactcgtcaagaaggc

SEQ ID NO: 38 - DNA, 25 bp, Artificial Sequence
  primer
tgagaaggtgaaagttcacagagga

SEQ ID NO: 39 - DNA, 25 bp, Artificial Sequence
  primer
cccacagtgagatttccatgaaggc

SEQ ID NO: 40 - DNA, 28 bp, Artificial Sequence
  primer
tcgataagcttgatatcgaattccgaag

SEQ ID NO: 41 - DNA, 25 bp, Artificial Sequence
  primer
tccgtttcagaaaagaggaggtcgg

SEQ ID NO: 42 - DNA, 25 bp, Artificial Sequence
  primer
gacaagcgttagtaggcacatatac

SEQ ID NO: 43 - DNA, 24 bp, Artificial Sequence
  primer
gctccaatttcccacaacattagt

SEQ ID NO: 44 - DNA, 20 bp, Artificial Sequence
  primer
atctacggggcttccctctt

SEQ ID NO: 45 - DNA, 20 bp, Artificial Sequence
  primer
gcaacaggtcaccagagtca

SEQ ID NO: 46 - DNA, 20 bp, Artificial Sequence
  primer
ctcctgtaccaccttaccgc

SEQ ID NO: 47 - DNA, 20 bp, Artificial Sequence
  primer
acaccgatgacgattcccac

SEQ ID NO: 48 - DNA, 22 bp, Artificial Sequence
  primer
tcaccatcttccaggagcgaga

SEQ ID NO: 49 - DNA, 21 bp, Artificial Sequence
  primer
gaaggccatgccagtgagctt

SEQ ID NO: 50 - DNA, 920 bp, Artificial Sequence
  poly A
ctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttcactagtctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttctgcagctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttc

(19) United States
(12) Patent Application Publication
(10) Pub. No.: US 2024/0081301 A1
(43) Pub. Date: Mar. 14, 2024

(54) GENETICALLY MODIFIED NON-HUMAN ANIMAL WITH HUMAN OR CHIMERIC FCRN

(71) Applicant: Biocytogen Pharmaceuticals (Beijing) Co., Ltd., Beijing (CN)

(72) Inventors: Chengzhang Shang, Beijing (CN); Chong Li, Beijing (CN); Chang Liu, Beijing (CN); Dirui Li, Beijing (CN)

(21) Appl. No.: 18/273,631

(22) PCT Filed: Jan. 29, 2022
(86) PCT No.: PCT/CN2022/075057

§ 371 Date: Jul. 21, 2023

(30) Foreign Application Priority Data
Feb. 2, 2021 (CN) 202110140651.0

(51) Int. Cl. A01K 67/027; A61K 49/00; C07K 14/74; C07K 16/28; C12N 15/85

(52) U.S. Cl. CPC A01K 67/0278; A61K 49/0008; C07K 14/70539; C07K 16/2818; C07K 16/2827; C12N 15/8509; A01K 2207/15; A01K 2217/052; A01K 2227/105; A01K 2267/01; A01K 2267/0325; C07K 2317/21; C07K 2317/24

(57) ABSTRACT

Provided are genetically modified animal expressing human or chimeric (e.g., humanized) FcRn, and methods of use thereof.

### CLAIM OF PRIORITY

This application claims the benefit of Chinese Patent Application No. CN202110140651.0, filed on Feb. 2, 2021. The entire contents of the foregoing are incorporated herein by reference.

### TECHNICAL FIELD

This disclosure relates to a genetically modified animal expressing human or chimeric (e.g., humanized) FcRn, and methods of use thereof.

### BACKGROUND

FcRn (neonatal Fc receptor for IgG), also known as FCGRT (Fc fragment of IgG receptor and transporter), collectively referred to as FcRn in different species, is an intracellular transport receptor for MHC class I molecules with a variety of key immune system functions. By binding to the Fc fragment of IgG, FcRn can prolong the half-life of IgG in the circulation and serum. Due to the key role of FcRn in controlling the pharmacokinetics (PK) of IgG, it has a very important role in the development and testing of therapeutic antibodies.

There is a need to develop an animal model with humanized FcRn.

### SUMMARY

This disclosure is related to an animal model with human FcRn or chimeric FcRn. The animal model can express human FcRn or chimeric FcRn (e.g., humanized FcRn) protein in its body. It can be used in the studies on the function of FcRn gene, and can be used in the screening and evaluation of various drugs.

In one aspect, the disclosure provides a genetically-modified, non-human animal whose genome comprises at least one chromosome comprising a sequence encoding a human or neonatal Fc receptor for IgG (FcRn). In some embodiments, the sequence encoding the human or chimeric FcRn is operably linked to an endogenous regulatory element at the endogenous FcRn gene locus in the at least one chromosome.

In one aspect, the disclosure is related to a genetically-modified, non-human animal whose genome comprises at least one chromosome comprising a sequence encoding a human or chimeric neonatal Fc receptor for IgG (FcRn).

In some embodiments, the sequence encoding the human or chimeric FcRn is operably linked to an endogenous regulatory element at the endogenous FcRn gene locus in the at least one chromosome.

In some embodiments, the sequence encoding the human or chimeric FcRn comprises a sequence encoding an amino acid sequence that is at least 70%, 75%, 80%, 85%, 90%, 95%, 99%, or 100% identical to SEQ ID NO: 2.

In some embodiments, the sequence encoding the human or chimeric FcRn is operably linked to an endogenous 5′-UTR (e.g., immediately after 5′-UTR).

In some embodiments, the animal is a mammal, e.g., a monkey, a rodent or a mouse.

In some embodiments, the animal is a mouse or a rat.

In some embodiments, the animal does not express endogenous FcRn or expresses a decreased level of endogenous FcRn as compared to that of an animal without genetic modification.

In some embodiments, the animal has one or more cells expressing human or chimeric FcRn.

In some embodiments, the sequence encoding the human or chimeric FcRn comprises a part of exon 1, all of exon2, all of exon 3, all of exon 4, all of exon 5 and a part of exon 6 of the human FcRn nucleotide sequence. In some embodiments, the part of exon 1 contains at least 50 bp of nucleotides, and the part of exon 6 contains at least 80 bp of the human FcRn nucleotide sequence.

In some embodiments, genetically-modified non-human animal comprises all of exon 1, a part of exon 2, all of exon 5, all of exon 6 and all of exon 7 of the non-human animal's endogenous FcRn gene.

In some embodiments, the human or chimeric FcRn protein comprises an amino acid sequence that are at least 70%, 75%, 80%, 85%, 90%, 91%, 92%, 93%, 94%, 95%, 96%, 97%, 98%, 99% or 100% identical to the amino acid sequences encoded by SEQ ID NO: 5, SEQ ID NO: 11 or SEQ ID NO: 23.

In some embodiments, the sequence encoding the human or chimeric FcRn contains a nucleotide sequence that are at least 70%, 75%, 80%, 85%, 90%, 91%, 92%, 93%, 94%, 95%, 96%, 97%, 98% or at least 99% identical to the nucleotide sequence shown in SEQ ID NO: 6, SEQ ID NO: 7, SEQ ID NO: 8, SEQ ID NO: 9, SEQ ID NO: 24, SEQ ID NO: 25, SEQ ID NO: 26, or SEQ ID NO: 27.

In some embodiments, the human or chimeric FcRn gene further comprises all of exon 1, a part of exon 2 and a part of exon 7 of the non-human animal's endogenous FcRn gene.

In one aspect, the disclosure is related to a genetically-modified, non-human animal, wherein the genome of the animal comprises an insertion of a sequence encoding a region of human FcRn or chimeric FcRn at an endogenous FcRn gene locus.

In some embodiments, the inserted sequence is operably linked to an endogenous regulatory element at the endogenous FcRn locus, and one or more cells of the animal express human FcRn or chimeric FcRn.

In some embodiments, the animal does not express endogenous FcRn or expresses a decreased level of endogenous FcRn as compared to that of an animal without genetic modification.

In some embodiments, the inserted sequence is located immediately after 5′-UTR at the endogenous FcRn locus.

In some embodiments, the animal has one or more cells expressing a chimeric FcRn having a humanized extracellular region, transmembrane region, and/or cytoplasmic region. In some embodiments, the humanized extracellular region comprises a sequence that is at least 50%, 60%, 70%, 80%, 90%, 95%, or 99% identical to the corresponding extracellular region of human FcRn.

In some embodiments, the human or chimeric FcRn comprises a sequence that is at least 70%, 75%, 80%, 85%, 90%, 91%, 92%, 93%, 94%, 95%, 96%, 97%, 98% or at least 99% identical to SEQ ID NO: 2.

In some embodiments, the genome of the animal comprises at least SEQ ID NO: 6, SEQ ID NO: 7, SEQ ID NO: 24, SEQ ID NO: 25, or a nucleotide sequence that is at least 70%, 75%, 80%, 85%, 90%, 91%, 92%, 93%, 94%, 95%, 96%, 97%, 98% or at least 99% identical to the nucleotide sequence set forth in SEQ ID NO: 6, SEQ ID NO: 7, SEQ ID NO: 24 or SEQ ID NO: 25.

In some embodiments, the animal further comprising a deletion of one or more nucleotide from the endogenous FcRn gene.

In some embodiments, the animal FcRn further comprises an endogenous FcRn 3′-UTR and a polyA sequence.

In some embodiments, the animal is heterozygous or homozygous with respect to the insertion at the endogenous FcRn gene locus.

In one aspect, the disclosure is related to a method for making a genetically-modified, non-human animal, comprising: inserting in at least one cell of the animal, at an endogenous FcRn gene locus, a sequence encoding at least a region of human FcRn gene.

In some embodiments, the sequence encoding the region of human FcRn gene comprises exon 1, exon 2, exon 3, exon 4, exon 5, exon 6, or a part thereof, of a human FcRn gene.

In some embodiments, the sequence encoding a region of human FcRn gene encodes a sequence that is at least 90% identical to SEQ ID NO: 2.

In some embodiments, the sequence encoding a region of human FcRn gene is 90% identical to SEQ ID NO: 5 or SEQ ID NO: 23.

In some embodiments, the sequence encoding a region of human FcRn gene is 100% identical to SEQ ID NO: 5 or SEQ ID NO: 23

In some embodiments, the method further comprises deleting one or more nucleotides of the endogenous FcRn gene.

In some embodiments, a part of exon 1, all of exon 2, all of exon 3, all of exon 4, all of exon 5 and a part of exon 6 of the human FcRn gene are inserted or substituted into the non-human animal's endogenous FcRn gene locus. In some embodiments, the part of exon 1 of the human FcRn gene comprises at least 50 bp contiguous human nucleotides. In some embodiments, the part of exon 6 of the human FcRn gene comprises at least 80 bp contiguous human nucleotides.

In some embodiments, a part of exon 2, exons 3-6 and a part of exon 7 of the endogenous mouse FcRn gene are replaced with a nucleotide sequence encoding the human or chimeric FcRn.

In some embodiments, a part of exon 2, all of exon 3, all of exon 4 of the endogenous mouse FcRn gene are replaced with a nucleotide sequence encoding the human or chimeric FcRn.

In one aspect, the disclosure is related to a non-human animal comprising at least one cell comprising a nucleotide sequence encoding a humanized FcRn polypeptide. In some embodiments, the humanized FcRn polypeptide comprises at least 50 contiguous amino acid residues that are identical to the corresponding contiguous amino acid sequence of a human FcRn. In some embodiments, the animal expresses the humanized FcRn.

In some embodiments, the humanized FcRn polypeptide has at least 10 contiguous amino acid residues that are identical to the corresponding contiguous amino acid sequence of a human FcRn extracellular region.

In some embodiments, the humanized FcRn polypeptide comprises a sequence that is at least 90%, 95%, or 99% identical to SEQ ID NO: 2.

In some embodiments, the nucleotide sequence is operably linked to an endogenous FcRn regulatory element of the animal (e.g., 5′-UTR).

In some embodiments, the humanized FcRn polypeptide comprises a humanized extracellular region, a humanized FcRn transmembrane region and/or a humanized FcRn cytoplasmic region.

In some embodiments, the nucleotide sequence is integrated to an endogenous FcRn gene locus of the animal.

In one aspect, the disclosure is related to A method of making a genetically-modified mouse cell that expresses a human FcRn or a chimeric FcRn, the method comprising: inserting at an endogenous mouse FcRn gene locus, a nucleotide sequence encoding a human FcRn or a chimeric FcRn, thereby generating a genetically-modified mouse cell that includes a nucleotide sequence that encodes the human FcRn or the chimeric FcRn. In some embodiments, the mouse cell expresses the human FcRn or the chimeric FcRn.

In some embodiments, the entire coding sequence of human FcRn gene is inserted at the endogenous mouse FcRn gene locus.

In some embodiments, the chimeric FcRn comprises: the extracellular region of human FcRn; and/or the transmembrane region; and/or the cytoplasmic region of mouse FcRn.

In some embodiments, the animal further comprises a sequence encoding an additional human or chimeric protein.

In some embodiments, the additional human or chimeric protein is H2-D, B2M, PD-1, PD-L1, CTLA4, B7H3, B7H4, CD47 or IL23A.

In some embodiments, the animal or mouse further comprises a sequence encoding an additional human or chimeric protein.

In some embodiments, the additional human or chimeric protein is H2-D, B2M, PD-1, PD-L1, CTLA4, B7H3, B7H4, CD47 or IL23A.

In one aspect, the disclosure is related to A method of determining effectiveness of a therapeutic agent targeting FcRn for the treatment of an immune-related disease, comprising: administering the therapeutic agent targeting FcRn to the animal of any one of claims 1-23 and 33-38; and determining the effects of the therapeutic agent targeting FcRn to the immune-related disease of the animal.

In some embodiments, the immune-related disease is an autoimmune disease.

In some embodiments, determining the effects of the therapeutic agent targeting FcRn to the immune-related disease of the animal comprises measuring the pharmacokinetic parameters of an antibody.

In some embodiments, the pharmacokinetic parameters include half-life (T_{1/2}), peak drug concentration (Cₘₐₓ), area under curve for the plasma concentration-time curve at 0-30 days (AUC₀₋₃₀), the area under curve for the plasma concentration-time curve from administration to theoretically extrapolated infinity (AUC_{0-obs}), the apparent volume of distribution (Vd), and the clearance rate (Cl).

In one aspect, the disclosure is related to A method for evaluating pharmacokinetics of an antibody, comprising administering the antibody to the animal of any one of any one of claims 1-23 and 33-38; and determining one or more pharmacokinetic parameters of the antibody.

In some embodiments, the pharmacokinetic parameters include half-life (T_{1/2}), peak drug concentration (Cₘₐₓ), area under curve for the plasma concentration-time curve at 0-30 days (AUC₀₋₃₀), the area under curve for the plasma concentration-time curve from administration to theoretically extrapolated infinity (AUC_{0-obs}), and the apparent volume of distribution (Vd), and the clearance rate (Cl).

In some embodiments, the antibody is a human or humanized antibody.

In one aspect, the disclosure is related to A method of determining effectiveness of a human or humanized antibody for the treatment of a disease, comprising
administering the antibody to the animal of any one of claims 1-23 and 33-38; and
determining the effects of the antibody on the disease.

In some embodiments, the disease is a tumor.

In some embodiments, the animal further comprises a sequence encoding a human or chimeric PD-1, PD-L1, CTLA-4, BTLA, CD27, CD28, CD40, CD47, CD137, CD154, TIGIT, TIM-3, GITR, or OX40.

In some embodiments, the antibody is an anti-PD-1 antibody or an anti-PD-L1 antibody.

In some embodiments, the human or chimeric FcRn nucleotide sequence comprises:
(A) all or part of the nucleotide sequence shown in SEQ ID NO: 5 or SEQ ID NO: 23;
(B) a nucleotide sequence that is at least 70%, 75%, 80%, 85%, 90%, 91%, 92%, 93%, 94%, 95%, 96%, 97%, 98% or at least 99% identical to the nucleotide sequence set forth in SEQ ID NO: 5 or SEQ ID NO: 23;
(C) a nucleotide sequence that is different from the nucleotide sequence set forth in SEQ ID NO: 5 or SEQ ID NO: 23 by no more than 10, 9, 8, 7, 6, 5, 4, 3, 2, or no more than 1 nucleotide;
(D) a nucleotide sequence that comprises a substitution, a deletion and/or insertion of one, two, three, four, five or more nucleotides to the nucleotide sequence set forth in SEQ ID NO: 5 or SEQ ID NO: 23.

In some embodiments, the non-human animal comprises an RNA that comprises:
(A) all or part of the nucleotide sequence shown in SEQ ID NO: 11;
(B) a nucleotide sequence that is at least 70%, 75%, 80%, 85%, 90%, 91%, 92%, 93%, 94%, 95%, 96%, 97%, 98% or at least 99% identical to the nucleotide sequence set forth in SEQ ID NO: 11;
(C) a nucleotide sequence that is different from the nucleotide sequence set forth in SEQ ID NO: 11 by no more than 10, 9, 8, 7, 6, 5, 4, 3, 2, or no more than 1 nucleotide;
(D) a nucleotide sequence that comprises a substitution, a deletion and /or insertion of one, two, three, four, five or more nucleotides to the nucleotide sequence set forth in SEQ ID NO: 11.

In one aspect, the disclosure relates to a protein comprising an amino acid sequence, wherein the amino acid sequence is one of the following:
(a) an amino acid sequence set forth in SEQ ID NO: 2;
(b) an amino acid sequence that is at least 90% identical to SEQ ID NO: 2;
(c) an amino acid sequence that is at least 91%, 92%, 93%, 94%, 95%, 96%, 97%, 98%, or 99% identical to SEQ ID NO: 2;
(d) an amino acid sequence that is different from the amino acid sequence set forth in SEQ ID NO: 2 by no more than 10, 9, 8, 7, 6, 5, 4, 3, 2 or 1 amino acid; and
(e) an amino acid sequence that comprises a substitution, a deletion and/or insertion of one, two, three, four, five or more amino acids to the amino acid sequence set forth in SEQ ID NO: 2.

In one aspect, the disclosure relates to a nucleic acid comprising a nucleotide sequence, wherein the nucleotide sequence is one of the following: (a) a sequence that encodes the protein as described herein; (b) SEQ ID NO: 3 or 21; (c) SEQ ID NO: 4 or 22; (d) SEQ ID NO: 5 or 23; (e) SEQ ID NO: 6; (f) SEQ ID NO: 7; (g) SEQ ID NO: 8; (h) SEQ ID NO: 9; (i) SEQ ID NO: 10; (j) a sequence that is at least 90% identical to SEQ ID NO: 3 or 21, SEQ ID NO: 4 or 22, SEQ ID NO: 5 or 23, SEQ ID NO: 6, SEQ ID NO: 7, SEQ ID NO: 8, SEQ ID NO: 9, or SEQ ID NO: 10; (k) a sequence that is at least 91%, 92%, 93%, 94%, 95%, 96%, 97%, 98%, or 99% identical to SEQ ID NO: 3 or 21, SEQ ID NO: 4 or 22, SEQ ID NO: 5 or 23, SEQ ID NO: 6, SEQ ID NO: 7, SEQ ID NO: 8, SEQ ID NO: 9, or SEQ ID NO: 10.

In one aspect, the disclosure provides a cell comprising the protein as described herein and/or the nucleic acid as described herein.

In one aspect, the disclosure provides an animal comprising the protein as described herein and/or the nucleic acid as described herein.

In one aspect, the disclosure is related to a screening method for a modulator, wherein the screening method comprises applying a modulator to an animal implanted with tumor cells to detect tumor inhibition, wherein the animal is selected from the animal as described herein.

In some embodiments, the modulator is selected from CAR-T and a drug.

In some embodiments, the method further comprises determining the pharmacokinetic parameters of the modulator.

In some embodiments, the pharmacokinetic parameters include half-life (T½), peak drug concentration (Cmax), area under curve for the plasma concentration-time curve at 0-30 days (AUC0-30), the area under curve for the plasma concentration-time curve from administration to theoretically extrapolated infinity (AUC0-obs), and the apparent volume of distribution (Vd), and the clearance rate (Cl).

In addition, the animal models prepared by the methods described herein can be used in drug screening, pharmacodynamics studies, testing treatments for FcRn related diseases. The disclosure also provides a powerful tool for studying the function of FcRn protein and a platform for screening drugs.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF THE DRAWINGS

FIG. 1 is a schematic diagram of the comparison between (1) mouse FcRn gene and (2) human FcRn gene locus (not to scale).

FIG. 2 is a schematic diagram of the humanization of mouse FcRn gene (not to scale) according to Example 1. chiExon1 corresponds to mExon 1. chiExon2 corresponds to the human or humanized FcRn sequence. chiExon3, chiExon4 and chiExon5 correspond to mExon5, mExon6 and mExon7, respectively.

FIG. 3 is a schematic diagram of FcRn gene targeting strategy and targeting vector design (not to scale) according to Example 1.

FIG. 4 shows the Southern Blot results of FcRn humanized mouse cells prepared according to Example 1. WT is the wild-type control. 1-G2, 2-A7, 2-A8, 2-D3, 3-B2, 3-D4, 3-G11, 4-B9 and 4-D9 are the clone numbers.

FIG. 5 is a schematic diagram of FRT recombination in FcRn humanized mice (not to scale) prepared according to Example 1.

FIG. 6 shows the PCR genotype identification results of FcRn humanized mice (F1 generation) prepared according to Example 1. WT means wild type control. H₂O means water control. PC means positive control. F1-01 is the mouse number.

FIG. 7 is a schematic diagram of the humanization of mouse FcRn gene (not to scale) according to Example 2. chiExon1, chiExon2, chiExon3, chiExon4, chiExon5, chiExon6 and chiExon7 correspond to mExon1, a combination of a part of mExon2 and a part of hExon2, hExon3, hExon4, hExon5, hExon6, and a combination of a part of mExon7 and a part of hExon7, respectively.

FIG. 8 is a schematic diagram of FcRn gene targeting strategy and targeting vector design (not to scale) according to Example 2.

FIG. 9 shows the Southern Blot results of FcRn humanized mouse cells prepared according to Example 2. WT is the wild-type control. 2-D03, 2-D08, 2-E05, 2-F09, 2-G08, 3-B12 and 3-H08 are the clone numbers.

FIG. 10 is a schematic diagram of FRT recombination in FcRn humanized mice (not to scale) prepared according to Example 2.

FIG. 11 shows the genotype identification results of FcRn humanized mice (F1 generation) prepared according to Example 2. WT means wild type control. H₂O means water control. PC means positive control. F1-01, F1-02, F1-03, F1-04, F1-05, F1-06, F1-07, F1-08 and F1-09 are the mouse numbers.

FIG. 12 shows the concentrations of anti-human PD-1 antibodies at various times in FcRn humanized mice and C57BL/6 wild-type mice.

FIG. 13 shows the concentrations of anti-human CTLA4 antibodies at various times in FcRn humanized mice and C57BL/6 wild-type mice.

FIG. 14 shows the amount of FcRn mRNA in kidney tissue of C57BL/6 wild-type mice (+/+) and FcRn humanized homozygous mice (H/H). H₂O is water control. GAPDH is glyceraldehyde-3-Phosphate dehydrogenase internal reference.

FIG. 15 shows the western blot results (protein expression levels) in C57BL/6 wild-type mice (+/+) and FcRn humanized homozygous mice (H/H) prepared according to Example 1 (H/H(V1)) and Example 2 (H/H(V2)). β-Actin is the β-actin protein reference.

FIG. 16 shows the western blot results (protein expression levels) in different tissues of FcRn humanized homozygous mice prepared according to Example 1 (H/H(V1)) and Example 2 (H/H(V2)). β-Actin is the β-actin protein reference.

FIG. 17 shows the western blot results (protein expression levels) in different tissues of FcRn humanized homozygous mice prepared according to Example 1.

FIG. 18 shows the concentrations of antibodies (including mutant antibodies) at various times in FcRn humanized homozygous mice prepared according to Example 1 (H/H(V1)) and C57BL/6 wild-type mice.

FIG. 19 shows the concentrations of anti-human antibody AB4 at various times in FcRn humanized homozygous mice prepared according to Example 1 (H/H(V1)).

FIG. 20: Alignment between mouse FcRn amino acid sequence (NP_034319.2; SEQ ID NO: 1) and human FcRn amino acid sequence (NP_004098.1; SEQ ID NO: 2).

### DETAILED DESCRIPTION

This disclosure relates to transgenic non-human animal with human or chimeric (e.g., humanized) FcRn (Neonatal Fc receptor for IgG), and methods of use thereof.

FcRn (Neonatal Fc receptor for IgG), also known as FCGRT (Fc fragment of IgG receptor and transporter) is an intracellular transport receptor for MHC class I molecules with a variety of key immune system functions. By binding to the Fc fragment of IgG, FcRn can prolong the half-life of IgG in the circulation and serum.

There are many receptors (Fc receptors, FcR) that can bind to the Fc region of an antibody. According to the type of the antibody they bind, they can be divided into FcgR (binding to IgG), FceR (binding to IgE), FcaR (binding to IgA) and FcA/MR (binding to IgA and IgM), each class including one or more molecules. FcRn is an FcR that specifically binds IgG, but is not technically in the same family as other FcgRs. Compared with other FcRs, the main role of FcRn is to maintain the homeostasis of IgG in serum. In addition, FcRn can also bind to serum albumin, protect it from degradation in lysosomes, and prolong its half-life in vivo. FcRn is an important factor in the self-regulation and conservation of IgG and albumin in the body.

The present disclosure provides a genetically modified animal expressing human or chimeric (e.g., humanized) FcRn, and methods of use thereof and demonstrates that the genetically modified animals as described herein can be properly used in drug screening. The humanized animal models have various important applications. For example, due to the presence of human or humanized genes, the animals can express or express in part of the proteins with human functions, so as to greatly reduce the differences in clinical trials between humans and animals, and provide the possibility of drug testing at animal levels.

### FcRn

FcRn is a neonatal Fc receptor. It is similar in structure to major histocompatibility complex I (MHC I). Human FcRn is very stringent regarding its specificity and binds human Fc, but not mouse, rat, bovine, or sheep Fc. The FcRn can bind to two sites of the IgG. Although mouse IgGs do not bind efficiently to human FcRn and therefore have a short half-life in humans, mouse IgG as an immune complex is capable of binding human FcRn and inducing signaling.

Most serum proteins have a short serum half-life (about 1-2 days). However, two types of serum proteins, namely albumin and antibodies of the IgG class, have greatly extended serum half-lives. For example, most subclasses of IgG have a half-life of about 10-20 days in humans. The Fc region of IgG is required for this extension of half-life. Thus, truncated IgG polypeptides carrying only the Fc region, and potentially also proteins carrying a short FcRn binding peptide sequence (FcBP), also show such extended serum half-life. Moreover, when the Fc region is fused with a fusion partner (e.g., a biologically active protein), this Fc fusion protein shows an extended serum half-life due to its interaction with FcRn.

The mechanism by which FcRn extends the serum half-life of IgG and IgG Fc fusion proteins is well established. FcRn is localized in the endosomal compartments of many cell types, including vascular endothelium. Serum proteins are constantly being endocytosed and directed to the early endosomal vesicles. FcRn is harbored primarily in this acidified vesicle. In this acidified environment, the Fc region binds FcRn, and the IgG/FcRn complex is then recycled either apically or basolaterally back to the plasma membrane, whereupon exposure to the neutral pH 7.2 extracellular environment results in its release into the circulation. In contrast, other endocytosed proteins that do not bind FcRn are not rescued, and thus continue through the endosomal route to catabolic elimination, resulting in their short half-life. The CH2-CH3-hinge region of the Fc region contains solvent exposed histidine residues, which when protonated, engage residues on FcRn with sufficient affinity to permit IgG to exploit the FcRn recycling pathway to escape catabolic elimination.

A detailed review of FcRn and its functions can be found in Martins, Joao Pedro, et al. “A comprehensive review of the neonatal Fc receptor and its application in drug delivery.” Pharmacology & therapeutics 161 (2016): 22-39 and Kuo, Timothy T., and Victoria G. Aveson. “Neonatal Fc receptor and IgG-based therapeutics.” MAbs. Vol. 3. No. 5. Taylor & Francis, 2011, which are incorporated by reference in its entirety.

In human genomes, FcRn gene (Gene ID: 2217) locus has 6 exons, exon 1, exon 2, exon 3, exon 4, and exon 5, and exon 6. The FcRn protein also has an extracellular region, a transmembrane region, and a cytoplasmic region. The nucleotide sequence for human FcRn mRNA is NM_004107.5, and the amino acid sequence for human FcRn is NP_004098.1 (SEQ ID NO: 2). The location for each exon and each region in human FcRn nucleotide sequence and amino acid sequence is listed below:

TABLE 1

| Human FcRn | NM_004107.5 | NP_004098.1 (365 aa) |
|---|---|---|
| (approximate location) | 1542 bp | (SEQ ID NO: 2) |
| Exon 1 | 1-208 | 1-24 |
| Exon 2 | 209-460 | 25-108 |
| Exon 3 | 461-736 | 109-200 |
| Exon 4 | 737-1006 | 201-290 |
| Exon 5 | 1007-1123 | 291-329 |
| Exon 6 | 1124-1542 | 330-365 |
| Signal peptide | 136-204 | 1-23 |
| Extracellular | 205-1026 | 24-297 |
| Transmembrane | 1027-1098 | 298-321 |
| Cytoplasmic | 1099-1230 | 322-365 |
| Donor region in | 136-1233 | 1-365 |
| Example 1 | (including the | (insertion of CDS) |
| | stop codon) | |
| Donor region in | 136-1233 | 1-365 |
| Example 2 | | (genomic sequence |
| | | replacement) |

The human FCRN gene (Gene ID: 2217) is located in Chromosome 19 of the human genome, which is located from 49512661 to 49526428 of NC_000019.10 (GRCh38.p13 (GCF_000001405.39)). The 5′-UTR is from 49513154 to 49513400, exon 1 is from 49513154 to 49513473, the first intron is from 49513474 to 49513881, exon 2 is from 49513882 to 49514133, the second intron is from 49514134 to 49514210, exon 3 is from 49514211 to 49514486, the third intron is from 49514487 to 49524506, exon 4 is from 49524507 to 49524776, the fourth intron is from 49524777 to 49525456, exon 5 is from 49525457 to 49525573, the fifth intron is from 49525574 to 49526009, exon 6 is from 49526010 to 49526333, the 3′-UTR is from 49526120 to 49526333, based on transcript NM_004107.5. All relevant information for human FcRn locus can be found in the NCBI website with Gene ID: 2217, which is incorporated by reference herein in its entirety.

In mice, FcRn gene (Gene ID: 14132) locus has 7 exons, exon 1, exon 2, exon 3, exon 4, and exon 5, exon 6 and exon 7. The mouse FcRn protein also has an extracellular region, a transmembrane region, and a cytoplasmic region. The nucleotide sequence for mouse FcRn mRNA is NM_010189.3, the amino acid sequence for mouse FcRn is NP_034319.2 (SEQ ID NO: 1). The location for each exon and each region in the mouse FcRn nucleotide sequence and amino acid sequence is listed below:

TABLE 2

| Mouse FCRN | NM_010189.3 | NP_034319.2 (365 aa) |
|---|---|---|
| (approximate location) | 1738 bp | (SEQ ID NO: 1) |
| Exon 1 | 1-183 | — |
| Exon 2 | 184-454 | 1-22 |
| Exon 3 | 455-712 | 23-108 |
| Exon 4 | 713-988 | 109-200 |
| Exon 5 | 989-1258 | 201-290 |
| Exon 6 | 1259-1375 | 291-329 |
| Exon 7 | 1376-1738 | 330-365 |
| Signal peptide | 388-450 | 1-21 |
| Extracellular | 451-1278 | 22-297 |
| Transmembrane | 1279-1350 | 298-321 |
| Cytoplasmic | 1351-1482 | 322-365 |
| Insert site in | 388-1482 | 1-365 |
| Example 1 | (insert site at | (insertion of CDS) |
| | start codon) | |
| Replaced region | 388-1482 | 1-365 |
| in Example 2 | (insert site at | (genomic sequence |
| | start codon) | replacement) |

The mouse FcRn gene (Gene ID: 14132) is located in Chromosome 7 of the mouse genome, which is located from 45092993 to 45103822 of NC_000073.6 (GRCm38.p6 (GCF_000001635.26)). The 5′-UTR is from 45103851 to 45103070, exon 1 is from 45103851 to 45103640, the first intron is from 45103639 to 45103274, exon 2 is from 45103273 to 45103003, the second intron is from 45103002 to 45102672, exon 3 is from 45102671 to 45102414, the third intron is from 45102413 to 45102186, exon 4 is from 45102185 to 45101910, the fourth intron is from 45101909 to 45095430, exon 5 is from 45095429 to 45095160, the fifth intron is from 45095159 to 45093899, exon 6 is from 45093898 to 45093782, the sixth intron is from 45093781 to 45093356, exon7 is from 45093355 to 45092990, the 3′-UTR is from 45093245 to 45092990, based on transcript NM_010189.3. All relevant information for mouse FCRN locus can be found in the NCBI website with Gene ID: 14132, which is incorporated by reference herein in its entirety.

FcRn genes, proteins, and locus of the other species are also known in the art. For example, the gene ID for FcRn in Rattus norvegicus is 29558, the gene ID for FcRn in Macaca fascicularis (crab-eating macaque) is 102128913, the gene ID for FcRn in Canis lupus familiaris (dog) is 476414, and the gene ID for FcRn in Bos taurus (cattle) is 338062. The relevant information for these genes (e.g., intron sequences, exon sequences, amino acid residues of these proteins) can be found, e.g., in NCBI database, which is incorporated by reference herein in its entirety.

The present disclosure provides human or chimeric (e.g., humanized) FcRn nucleotide sequence and/or amino acid sequences. In some embodiments, the entire sequence of mouse exon 1, exon 2, exon 3, exon 4, exon 5, exon 6, exon 7, signal peptide, extracellular region, transmembrane region, and/or cytoplasmic region are replaced by the corresponding human sequence. In some embodiments, a “region” or “portion” of mouse exon 1, exon 2, exon 3, exon 4, exon 5, exon 6, exon 7, signal peptide, extracellular region, transmembrane region, and/or cytoplasmic region are replaced by the corresponding human sequence. The term “region” or “portion” can refer to at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 250, 300, 350, 400, 500, or 600 nucleotides, or at least 1, 2, 3, 4, 5, 6, 7, 8, 9, 10, 20, 30, 40, 50, 60, 70, 80, 90, 100, 110, 120, 130, 140, 150, 160, 170, 180, 190, or 200 amino acid residues. In some embodiments, the “region” or “portion” can be at least 50%, 55%, 60%, 65%, 70%, 75%, 80%, 85%, 90%, 95%, or 99% identical to exon 1, exon 2, exon 3, exon 4, exon 5, exon 6, exon 7, signal peptide, extracellular region, transmembrane region, or cytoplasmic region. In some embodiments, a region, a portion, or the entire sequence of mouse exon 1, exon 2, exon 3, exon 4, exon 5, exon 6, and/or exon 7 are replaced by the human exon 1, exon 2, exon 3, exon 4, exon 5, and/or exon 6 sequence.

In some embodiments, the present disclosure also provides a chimeric (e.g., humanized) FcRn nucleotide sequence and/or amino acid sequences, wherein in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from mouse FcRn gene, mouse FcRn amino acid sequence (e.g., SEQ ID NO: 1), or a portion thereof (e.g., exon 1, exon 2, exon 3, exon 4, exon 5, exon 6, and/or exon 7); and in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from human FcRn gene sequence, human FcRn amino acid sequence (e.g., SEQ ID NO: 2), or a portion thereof (e.g., exon 1, exon 2, exon 3, exon 4, exon 5, and/or exon 6).

In some embodiments, the nucleic acids as described herein are operably linked to a promotor or regulatory element, e.g., an endogenous mouse FcRn promotor, an inducible promoter, an enhancer, and/or mouse or human regulatory elements.

In some embodiments, the nucleic acids as described herein are operably linked to a Woodchuck Hepatitis Virus (WHP) Posttranscriptional Regulatory Element (WPRE). In some embodiments, the nucleic acids as described herein are operably linked to a polyA (polyadenylation) signal sequence. In some embodiments, the polyA (polyadenylation) signal sequence has a sequence that is at least 70%, 80%, 90%, or 95% identical to SEQ ID NO: 50.

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are different from a portion of or the entire mouse FcRn nucleotide sequence (e.g., exon 1, exon 2, exon 3, exon 4, exon 5, exon 6, and/or exon 7).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire mouse FcRn nucleotide sequence (e.g., exon 1, exon 2, exon 3, exon 4, exon 5, exon 6, exon 7).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from a portion of or the entire human FcRn nucleotide sequence (e.g., exon 1, exon 2, exon 3, exon 4, exon 5, exon 6).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire human FcRn nucleotide sequence (e.g., exon 1, exon 2, exon 3, exon 4, exon 5, exon 6).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire mouse FcRn amino acid sequence (e.g., exon 1, exon 2, exon 3, exon 4, exon 5, exon 6, exon 7 or SEQ ID NO: 1).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire mouse FcRn amino acid sequence (e.g., exon 1, exon 2, exon 3, exon 4, exon 5, exon 6, exon 7 or SEQ ID NO: 1).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire human FcRn amino acid sequence (e.g., exon 1, exon 2, exon 3, exon 4, exon 5, exon 6, or SEQ ID NO: 2).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire human FcRn amino acid sequence (e.g., exon 1, exon 2, exon 3, exon 4, exon 5, exon 6, or SEQ ID NO: 2).

The present disclosure also provides a humanized FcRn mouse amino acid sequence, wherein the amino acid sequence is selected from the group consisting of:
a) an amino acid sequence shown in SEQ ID NO: 2;
b) an amino acid sequence having a homology of at least 90% with or at least 90% identical to the amino acid sequence shown in SEQ ID NO: 2;
c) an amino acid sequence encoded by a nucleic acid sequence, wherein the nucleic acid sequence is able to hybridize to a nucleotide sequence encoding the amino acid shown in SEQ ID NO: 2 under a low stringency condition or a strict stringency condition;
d) an amino acid sequence having a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the amino acid sequence shown in SEQ ID NO: 2;
e) an amino acid sequence that is different from the amino acid sequence shown in SEQ ID NO: 2 by no more than 10, 9, 8, 7, 6, 5, 4, 3, 2 or no more than 1 amino acid; or
f) an amino acid sequence that comprises a substitution, a deletion and/or insertion of one or more amino acids to the amino acid sequence shown in SEQ ID NO: 2.

The present disclosure also relates to a FcRn nucleic acid (e.g., DNA or RNA) sequence, wherein the nucleic acid sequence can be selected from the group consisting of:
a) a nucleic acid sequence as shown in SEQ ID NO: 5 or 23, or a nucleic acid sequence encoding a homologous FcRn amino acid sequence of a humanized mouse;
b) a nucleic acid sequence that is shown in SEQ ID NO: 5 or 23;
c) a nucleic acid sequence that is able to hybridize to the nucleotide sequence as shown in SEQ ID NO: 5 or 23 under a low stringency condition or a strict stringency condition;
d) a nucleic acid sequence that has a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the nucleotide sequence as shown in SEQ ID NO: 5 or 23;
e) a nucleic acid sequence that encodes an amino acid sequence, wherein the amino acid sequence has a homology of at least 90% with or at least 90% identical to the amino acid sequence shown in SEQ ID NO: 2;
f) a nucleic acid sequence that encodes an amino acid sequence, wherein the amino acid sequence has a homology of at least 90%, 91%, 92%, 93%, 94%, 95%, 96%, 97%, 98%, or 99% with, or at least 90%, 91%, 92%, 93%, 94%, 95%, 96%, 97%, 98%, or 99% identical to the amino acid sequence shown in SEQ ID NO: 2;
g) a nucleic acid sequence that encodes an amino acid sequence, wherein the amino acid sequence is different from the amino acid sequence shown in SEQ ID NO: 2 by no more than 10, 9, 8, 7, 6, 5, 4, 3, 2 or no more than 1 amino acid; and/or
h) a nucleic acid sequence that encodes an amino acid sequence, wherein the amino acid sequence comprises a substitution, a deletion and/or insertion of one or more amino acids to the amino acid sequence shown in SEQ ID NO: 2.

The present disclosure further relates to a FcRn genomic DNA sequence of a humanized mouse. The DNA sequence is obtained by a reverse transcription of the mRNA obtained by transcription thereof is consistent with or complementary to the DNA sequence homologous to the sequence shown in SEQ ID NO: 5 or 23.

The disclosure also provides an amino acid sequence that has a homology of at least 90% with, or at least 90% identical to the sequence shown in SEQ ID NO: 2, and has protein activity.

In some embodiments, the homology with the sequence shown in SEQ ID NO: 2 is at least about 90%, 91%, 92%, 93%, 94%, 95%, 96%, 97%, 98%, or at least 99%. In some embodiments, the foregoing homology is at least about 60%, 61%, 62%, 63%, 64%, 65%, 66%, 67%, 68%, 69%, 70%, 71%, 72%, 73%, 74%, 75%, 80%, or 85%.

In some embodiments, the percentage identity with the sequence shown in SEQ ID NO: 2 is at least about 90%, 91%, 92%, 93%, 94%, 95%, 96%, 97%, 98%, or at least 99%. In some embodiments, the foregoing percentage identity is at least about 60%, 61%, 62%, 63%, 64%, 65%, 66%, 67%, 68%, 69%, 70%, 71%, 72%, 73%, 74%, 75%, 80%, or 85%.

The disclosure also provides a nucleotide sequence that has a homology of at least 90%, or at least 90% identical to the sequence shown in SEQ ID NO: 5 or 23, and encodes a polypeptide that has protein activity. In some embodiments, the homology with the sequence shown in SEQ ID NO: 5 or 23 is at least about 90%, 91%, 92%, 93%, 94%, 95%, 96%, 97%, 98%, or at least 99%. In some embodiments, the foregoing homology is at least about 50%, 55%, 60%, 65%, 66%, 67%, 68%, 69%, 70%, 71%, 72%, 73%, 74%, 75%, 80%, or 85%.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein. In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, or 200 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

The percentage of residues conserved with similar physicochemical properties (percent homology), e.g. leucine and isoleucine, can also be used to measure sequence similarity. Families of amino acid residues having similar physicochemical properties have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The homology percentage, in many cases, is higher than the identity percentage.

Cells, tissues, and animals (e.g., mouse) are also provided that comprise the nucleotide sequences as described herein, as well as cells, tissues, and animals (e.g., mouse) that express human or chimeric (e.g., humanized) FcRn from an endogenous FcRn locus.

### Genetically Modified Animals

As used herein, the term “genetically-modified non-human animal” refers to a non-human animal having exogenous DNA in at least one chromosome of the animal's genome. In some embodiments, at least one or more cells, e.g., at least 1%, 2%, 3%, 4%, 5%, 10%, 20%, 30%, 40%, 50% of cells of the genetically-modified non-human animal have the exogenous DNA in its genome. The cell having exogenous DNA can be various kinds of cells, e.g., an endogenous cell, a somatic cell, an immune cell, a T cell, a B cell, an antigen presenting cell, a macrophage, a dendritic cell, a germ cell, a blastocyst, or an endogenous tumor cell. In some embodiments, genetically-modified non-human animals are provided that comprise a modified endogenous FcRn locus that comprises an exogenous sequence (e.g., a human sequence), e.g., a replacement of one or more non-human sequences with one or more human sequences. The animals are generally able to pass the modification to progeny, i.e., through germline transmission.

As used herein, the term “chimeric gene” or “chimeric nucleic acid” refers to a gene or a nucleic acid, wherein two or more portions of the gene or the nucleic acid are from different species, or at least one of the sequences of the gene or the nucleic acid does not correspond to the wildtype nucleic acid in the animal. In some embodiments, the chimeric gene or chimeric nucleic acid has at least one portion of the sequence that is derived from two or more different sources, e.g., sequences encoding different proteins or sequences encoding the same (or homologous) protein of two or more different species. In some embodiments, the chimeric gene or the chimeric nucleic acid is a humanized gene or humanized nucleic acid.

As used herein, the term “chimeric protein” or “chimeric polypeptide” refers to a protein or a polypeptide, wherein two or more portions of the protein or the polypeptide are from different species, or at least one of the sequences of the protein or the polypeptide does not correspond to wildtype amino acid sequence in the animal. In some embodiments, the chimeric protein or the chimeric polypeptide has at least one portion of the sequence that is derived from two or more different sources, e.g., same (or homologous) proteins of different species. In some embodiments, the chimeric protein or the chimeric polypeptide is a humanized protein or a humanized polypeptide.

In some embodiments, the chimeric gene or the chimeric nucleic acid is a humanized FcRn gene or a humanized FcRn nucleic acid. In some embodiments, at least one or more portions of the gene or the nucleic acid is from the human FcRn gene, at least one or more portions of the gene or the nucleic acid is from a non-human FcRn gene. In some embodiments, the gene or the nucleic acid comprises a sequence that encodes a FcRn protein. The encoded FcRn protein is functional or has at least one activity of the human FcRn protein or the non-human FcRn protein, e.g., binding with human or non-human IgG.

In some embodiments, the chimeric protein or the chimeric polypeptide is a humanized FcRn protein or a humanized FcRn polypeptide. In some embodiments, at least one or more portions of the amino acid sequence of the protein or the polypeptide is from a human FcRn protein, and at least one or more portions of the amino acid sequence of the protein or the polypeptide is from a non-human FcRn protein. The humanized FcRn protein or the humanized FcRn polypeptide is functional or has at least one activity of the human FcRn protein or the non-human FcRn protein.

The genetically modified non-human animal can be various animals, e.g., a mouse, rat, rabbit, pig, bovine (e.g., cow, bull, buffalo), deer, sheep, goat, chicken, cat, dog, ferret, primate (e.g., marmoset, rhesus monkey). For the non-human animals where suitable genetically modifiable embryonic stem (ES) cells are not readily available, other methods are employed to make a non-human animal comprising the genetic modification. Such methods include, e.g., modifying a non-ES cell genome (e.g., a fibroblast or an induced pluripotent cell) and employing nuclear transfer to transfer the modified genome to a suitable cell, e.g., an oocyte, and gestating the modified cell (e.g., the modified oocyte) in a non-human animal under suitable conditions to form an embryo. These methods are known in the art, and are described, e.g., in A. Nagy, et al., “Manipulating the Mouse Embryo: A Laboratory Manual (Third Edition),” Cold Spring Harbor Laboratory Press, 2003, which is incorporated by reference herein in its entirety.

In one aspect, the animal is a mammal, e.g., of the superfamily Dipodoidea or Muroidea. In some embodiments, the genetically modified animal is a rodent. The rodent can be selected from a mouse, a rat, and a hamster. In some embodiments, the genetically modified animal is from a family selected from Calomyscidae (e.g., mouse-like hamsters), Cricetidae (e.g., hamster, New World rats and mice, voles), Muridae (true mice and rats, gerbils, spiny mice, crested rats), Nesomyidae (climbing mice, rock mice, with-tailed rats, Malagasy rats and mice), Platacanthomyidae (e.g., spiny dormice), and Spalacidae (e.g., mole rates, bamboo rats, and zokors). In some embodiments, the genetically modified rodent is selected from a true mouse or rat (family Muridae), a gerbil, a spiny mouse, and a crested rat. In some embodiments, the non-human animal is a mouse.

In some embodiments, the animal is a mouse of a C57BL strain selected from C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, and C57BL/Ola. In some embodiments, the mouse is a 129 strain selected from the group consisting of a strain that is 129P1, 129P2, 129P3, 129X1, 129S1 (e.g., 129S1/SV, 129S1/SvIm), 129S2, 129S4, 129S5, 129S9/SvEvH, 129S6 (129/SvEvTac), 129S7, 129S8, 129T1, 129T2. These mice are described, e.g., in Festing et al., Revised nomenclature for strain 129 mice, Mammalian Genome 10: 836 (1999); Auerbach et al., Establishment and Chimera Analysis of 129/SvEv- and C57BL/6-Derived Mouse Embryonic Stem Cell Lines (2000), both of which are incorporated herein by reference in the entirety. In some embodiments, the genetically modified mouse is a mix of the 129 strain and the C57BL/6 strain. In some embodiments, the mouse is a mix of the 129 strains, or a mix of the BL/6 strains. In some embodiments, the mouse is a BALB strain, e.g., BALB/c strain. In some embodiments, the mouse is a mix of a BALB strain and another strain. In some embodiments, the mouse is from a hybrid line (e.g., 50% BALB/c-50% 12954/Sv; or 50% C57BL/6-50% 129).

In some embodiments, the animal is a rat. The rat can be selected from a Wistar rat, an LEA strain, a Sprague Dawley strain, a Fischer strain, F344, F6, and Dark Agouti. In some embodiments, the rat strain is a mix of two or more strains selected from the group consisting of Wistar, LEA, Sprague Dawley, Fischer, F344, F6, and Dark Agouti.

The animal can have one or more other genetic modifications, and/or other modifications, that are suitable for the particular purpose for which the humanized FcRn animal is made. For example, suitable mice for maintaining a xenograft (e.g., a human cancer or tumor), can have one or more modifications that compromise, inactivate, or destroy the immune system of the non-human animal in whole or in part. Compromise, inactivation, or destruction of the immune system of the non-human animal can include, for example, destruction of hematopoietic cells and/or immune cells by chemical means (e.g., administering a toxin), physical means (e.g., irradiating the animal), and/or genetic modification (e.g., knocking out one or more genes). Non-limiting examples of such mice include, e.g., NOD mice, SCID mice, NOD/SCID mice, IL2Rγ knockout mice, NOD/SCID/γcnull mice (Ito, M. et al., NOD/SCID/γcnull mouse: an excellent recipient mouse model for engraftment of human cells, Blood 100 (9): 3175-3182, 2002), nude mice, and Rag1 and/or Rag2 knockout mice. These mice can optionally be irradiated, or otherwise treated to destroy one or more immune cell type. Thus, in various embodiments, a genetically modified mouse is provided that can include a humanization of at least a portion of an endogenous non-human FcRn locus, and further comprises a modification that compromises, inactivates, or destroys the immune system (or one or more cell types of the immune system) of the non-human animal in whole or in part. In some embodiments, modification is, e.g., selected from the group consisting of a modification that results in NOD mice, SCID mice, NOD/SCID mice, IL-2Rγ knockout mice, NOD/SCID/γc null mice, nude mice, Rag1 and/or Rag2 knockout mice, and a combination thereof. These genetically modified animals are described, e.g., in US20150106961, which is incorporated herein by reference in its entirety. In some embodiments, the mouse can include a replacement of all or part of mature FcRn coding sequence with human mature FcRn coding sequence or an insertion of human mature FcRn coding sequence or chimeric FcRn coding sequence.

Genetically modified non-human animals that comprise a modification of an endogenous non-human FcRn locus. In some embodiments, the modification can comprise a human nucleic acid sequence encoding at least a portion of a mature FcRn protein (e.g., at least 10%, 20%, 30%, 40%, 50%, 60%, 70%, 80%, 90%, 95%, 96%, 97%, 98%, or 99% identical to the mature FcRn protein sequence). Although genetically modified cells are also provided that can comprise the modifications described herein (e.g., ES cells, somatic cells), in many embodiments, the genetically modified non-human animals comprise the modification of the endogenous FcRn locus in the germline of the animal.

Genetically modified animals can express a human FcRn and/or a chimeric (e.g., humanized) FcRn from endogenous loci, wherein the endogenous mouse FcRn gene has been replaced with a human FcRn gene and/or a nucleotide sequence that encodes a region of human FcRn sequence or an amino acid sequence that is at least 10%, 20%, 30%, 40%, 50%, 60%, 70%, 80%, 90%, 95%, 96%, 97%, 98%, or 99% identical to the human FcRn sequence. In various embodiments, an endogenous non-human FcRn locus is modified in whole or in part to comprise human nucleic acid sequence encoding at least one protein-coding sequence of a mature FcRn protein.

In some embodiments, the genetically modified animals (e.g., mice) can express a human FcRn and/or a chimeric (e.g., humanized) FcRn in the liver, lung, spleen, small intestine and kidney tissues.

In some embodiments, the genetically modified mice express the human FcRn and/or chimeric FcRn (e.g., humanized FcRn) from endogenous loci that are under control of mouse promoters and/or mouse regulatory elements. The replacement(s) at the endogenous mouse loci provide non-human animals that express human FcRn or chimeric FcRn (e.g., humanized FcRn) in appropriate cell types and in a manner that does not result in the potential pathologies observed in some other transgenic mice known in the art. The human FcRn or the chimeric FcRn (e.g., humanized FcRn) expressed in animal can maintain one or more functions of the wildtype mouse or human FcRn in the animal. For example, human or non-human FcRn ligands (e.g., IgG and/or albumin) can bind to the expressed FcRn. Furthermore, in some embodiments, the animal does not express endogenous FcRn. As used herein, the term “endogenous FcRn” refers to FcRn protein that is expressed from an endogenous FcRn nucleotide sequence of the non-human animal (e.g., mouse) before any genetic modification.

The genome of the animal can comprise a sequence encoding an amino acid sequence that is at least 70%, 75%, 80%, 85%, 90%, 95%, 99%, or 100% identical to human FcRn (SEQ ID NO: 2).

The genome of the genetically modified animal can comprise a replacement at an endogenous FcRn gene locus of a sequence encoding a region of endogenous FcRn with a sequence encoding a corresponding region of human FcRn. In some embodiments, the sequence that is replaced is any sequence within the endogenous FcRn gene locus, e.g., exon 1, exon 2, exon 3, exon 4, exon 5, exon 6, exon 7, 5′-UTR, 3′-UTR, the first intron, the second intron, the third intron, the fourth intron, the fifth intron, the sixth intron, the extracellular region, the cytoplasmic region, etc. In some embodiments, the sequence that is replaced is within the exon 2 of the endogenous FcRn gene.

In some embodiments, a sequence that encodes an amino acid sequence (e.g., human FcRn or chimeric FcRn) is inserted after 5′-UTR (e.g., immediately after 5′-URT), or immediately before the start codon (e.g., within 10, 20, 30, 40, 50, 60, 70, 80, 90, or 100 nucleic acids). The start codon is the first codon of a messenger RNA (mRNA) transcript translated by a ribosome. The start codon always codes for methionine in eukaryotes and a modified Met (fMet) in prokaryotes. The most common start codon is ATG (or AUG in mRNA).

In some embodiments, the inserted sequence further comprises a stop codon (e.g., TAG, TAA, TGA). The stop codon (or termination codon) is a nucleotide triplet within messenger RNA that signals a termination of translation into proteins. Thus, the endogenous sequence after the stop codon will not be translated into proteins. In some embodiments, at least one exon of (e.g., exon 1, exon 2, exon 3, exon 4, exon 5, exon 6, and/or exon 7) of the endogenous FcRn gene are not translated into proteins.

The genetically modified animal can have one or more cells expressing a human or chimeric FcRn (e.g., humanized FcRn) having an extracellular region and a cytoplasmic region, wherein the extracellular region comprises a sequence that is at least 50%, 60%, 70%, 80%, 90%, 95%, 99% identical to one of the extracellular region of human FcRn. In some embodiments, the extracellular region of the humanized FcRn has a sequence that has at least 10, 20, 30, 40, 50, 60, 70, 80, 90, 100, 110, 120, 130, 140, 150, 160, 170, or 180 amino acids (e.g., contiguously or non-contiguously) that are identical to one of the extracellular region of human FcRn. In some embodiments, the genome of the genetically modified animal comprises a sequence encoding an amino acid sequence that corresponds to part or the entire sequence of exon 1, exon 2, exon 3, exon 4, exon 5, and/or exon 6 of human FcRn, or part or the entire sequence of the extracellular region of human FcRn (with or without signal peptide).

In some embodiments, the non-human animal can have, at an endogenous FcRn gene locus, a nucleotide sequence encoding a chimeric human/non-human FcRn polypeptide, wherein a human portion of the chimeric human/non-human FcRn polypeptide comprises a portion of the human FcRn extracellular region, and wherein the animal expresses a functional FcRn on a surface of a cell of the animal. The human portion of the chimeric human/non-human FcRn polypeptide can comprise a portion of exon 1, exon 2, exon 3, exon 4, exon 5, and/or exon 6 of human FcRn. In some embodiments, the human portion of the chimeric human/non-human FcRn polypeptide can comprise a sequence that is at least 80%, 85%, 90%, 95%, or 99% identical to SEQ ID NO: 2.

In some embodiments, the non-human animal genome also includes other genetic modifications. In some embodiments, the other genes include one of human PD-1, PD-L1, CTLA4, LAG3, IL4, IL6, or CCR4 genes, or a combination of two or more. In some embodiments, the other genes include one or more of H2-D, B2M, PD-1, PD-L1, CTLA4, B7H3, B7H4, CD47 and IL23A.

In some embodiments, the nucleotide sequence of the humanized FcRn gene includes one of the following groups:
(1) all or part of the nucleotide sequence shown in SEQ ID NO: 5 or 23;
(2) at least 60%, 65%, 70%, 75%, 80%, 85%, 90%, 91%, 92%, 93% identity with the nucleotide sequence shown in SEQ ID NO: 5 or 23, 94%, 95%, 96%, 97%, 98% or at least 99%;
(3) no more than 10, 9, 8, 7, 6, 5, 4, 3, 2 or no more than 1 nucleotide difference from the nucleotide sequence shown in SEQ ID NO: 5 or 23; or
(4) the nucleotide sequence shown in the nucleotide sequence shown in SEQ ID NO: 5 or 23, including substitution, deletion and/or insertion of one or more nucleotides.

In some embodiments, the humanized FcRn gene further comprises an auxiliary sequence, which is connected after the human FcRn gene. Further preferably, the auxiliary sequence is selected from a stop codon, a flip sequence or a knockout sequence. More preferably, the auxiliary sequence is 3′UTR and/or polyA of a non-human animal.

In some embodiments, the non-human animal can have transcribed mRNA sequence including one of the following groups:
(1) all or part of the nucleotide sequence shown in SEQ ID NO: 11;
(2) at least 60%, 65%, 70%, 75%, 80%, 85%, 90%, 91%, 92%, 93% identity with the nucleotide sequence shown in SEQ ID NO: 11, 94%, 95%, 96%, 97%, 98% or at least 99%;
(3) no more than 10, 9, 8, 7, 6, 5, 4, 3, 2 or no more than 1 nucleotide difference from the nucleotide sequence shown in SEQ ID NO: 11; or
(4) the nucleotide sequence shown in the nucleotide sequence shown in SEQ ID NO: 11, including substitution, deletion and/or insertion of one or more nucleotides.

In some embodiments, the humanized FcRn gene also includes a specific inducer or repressor. Further preferably, the specific inducer or repressor can be a conventional inducing or repressing substance.

In a specific embodiment of the present invention, the specific inducer is selected from the tetracycline system (Tet-Off System/Tet-On System) or the tamoxifen system (Tamoxifen System).

In some embodiments, the non-human portion of the chimeric human/non-human FcRn polypeptide comprises the transmembrane and/or cytoplasmic region of an endogenous non-human FcRn polypeptide.

Furthermore, the genetically modified animal can be heterozygous with respect to the replacement or insertion at the endogenous FcRn locus, or homozygous with respect to the replacement or insertion at the endogenous FcRn locus.

In some embodiments, the genetically modified animal (e.g., a rodent) comprises a humanization of an endogenous FcRn gene, wherein the humanization comprises a replacement at the endogenous rodent FcRn locus of a nucleic acid comprising an exon of a FcRn gene with a nucleic acid sequence comprising at least one exon of a human FcRn gene to form a modified FcRn gene.

In some embodiments, the genetically modified animal (e.g., a rodent) comprises an insertion at the endogenous rodent FcRn locus of a nucleic acid sequence comprising at least one exon of a human FcRn gene to form a modified FcRn gene.

In some embodiments, the expression of the modified FcRn gene is under control of regulatory elements at the endogenous FcRn locus. In some embodiments, the modified FcRn gene is operably linked to a WPRE element.

In some embodiments, the humanized FcRn locus lacks a human FcRn 5′-UTR. In some embodiment, the humanized FcRn locus comprises a rodent (e.g., mouse) 5′-UTR. In some embodiments, the humanization comprises a human 3′-UTR. In appropriate cases, it may be reasonable to presume that the mouse and human FcRn genes appear to be similarly regulated based on the similarity of their 5′-flanking sequence. As shown in the present disclosure, humanized FcRn mice that comprise an insertion at an endogenous mouse FcRn locus, which retain mouse regulatory elements but comprise a humanization of FcRn encoding sequence, do not exhibit obvious pathologies. Both genetically modified mice that are heterozygous or homozygous for humanized FcRn are grossly normal.

The present disclosure further relates to a non-human mammal generated through the method mentioned above. In some embodiments, the genome thereof contains human gene(s).

In some embodiments, the non-human mammal is a rodent, and preferably, the non-human mammal is a mouse.

In some embodiments, the non-human mammal expresses a protein encoded by a humanized FcRn gene.

In addition, the present disclosure also relates to a tumor bearing non-human mammal model, characterized in that the non-human mammal model is obtained through the methods as described herein. In some embodiments, the non-human mammal is a rodent (e.g., a mouse).

The present disclosure further relates to a cell or cell line, or a primary cell culture thereof derived from the non-human mammal or an offspring thereof, or the tumor bearing non-human mammal; the tissue, organ or a culture thereof derived from the non-human mammal or an offspring thereof, or the tumor bearing non-human mammal; and the tumor tissue derived from the non-human mammal or an offspring thereof when it bears a tumor, or the tumor bearing non-human mammal.

The present disclosure also provides non-human mammals produced by any of the methods described herein. In some embodiments, a non-human mammal is provided; and the genetically modified animal contains the DNA encoding human or humanized FcRn in the genome of the animal.

In some embodiments, the non-human mammal comprises the genetic construct as described herein (e.g., gene construct as shown in FIG. 3 or 8). In some embodiments, a non-human mammal expressing human or humanized FcRn is provided. In some embodiments, the tissue-specific expression of human or humanized FcRn protein is provided.

In some embodiments, the expression of human or humanized FcRn in a genetically modified animal is controllable, as by the addition of a specific inducer or repressor substance.

Non-human mammals can be any non-human animal known in the art and which can be used in the methods as described herein. Preferred non-human mammals are mammals, (e.g., rodents). In some embodiments, the non-human mammal is a rodent, e.g., a mouse.

Genetic, molecular and behavioral analyses for the non-human mammals described above can performed. The present disclosure also relates to the progeny produced by the non-human mammal provided by the present disclosure mated with the same or other genotypes.

The present disclosure also provides a cell line or primary cell culture derived from the non-human mammal or a progeny thereof. A model based on cell culture can be prepared, for example, by the following methods. Cell cultures can be obtained by way of isolation from a non-human mammal, alternatively cell can be obtained from the cell culture established using the same constructs and the standard cell transfection techniques. The integration of genetic constructs containing DNA sequences encoding human FcRn protein or chimeric FcRn protein can be detected by a variety of methods.

There are many analytical methods that can be used to detect exogenous DNA, including methods at the level of nucleic acid (including the mRNA quantification approaches using reverse transcriptase polymerase chain reaction (RT-PCR) or Southern blotting, and in situ hybridization) and methods at the protein level (including histochemistry, immunoblot analysis and in vitro binding studies). In addition, the expression level of the gene of interest can be quantified by ELISA techniques well known to those skilled in the art. Many standard analysis methods can be used to complete quantitative measurements. For example, transcription levels can be measured using RT-PCR and hybridization methods including RNase protection, Southern blot analysis, RNA dot analysis (RNAdot) analysis. Immunohistochemical staining, flow cytometry, Western blot analysis can also be used to assess the presence of human or humanized FcRn protein.

In some embodiments, the humanized FcRn protein comprises all or part of the amino acid sequence encoded by exons 1 to 6 of the human FcRn gene. In some embodiments, the humanized FcRn protein comprises all or part of the human FcRn amino acid sequence encoded by any one, two, three or more, two consecutive or three or more consecutive exons of the human FcRn gene. In some embodiments, the humanized FcRn protein comprises the entire amino acid sequence encoded by exons 1-6 of the human FcRn gene.

In some embodiments, the humanized FcRn protein comprises the amino acid sequence encoded by a part of exon 1, all of exons 2 to 5 and a part of exon 6 of the human FcRn gene. In some embodiments, the part of exon 1 of the human FcRn gene contains at least the start codon to the last nucleotide of exon 1. In some embodiments, the part of exon 1 contains at least 50 bp (for example, 50, 55, 60, 65, 66, 67, 68, 69, 70, 71, 72, 73 bp) nucleotides. In some embodiments, the part of exon 1 contains 73bp nucleotides. In some embodiments, the part of exon 6 contains at least from the first nucleotide of exon 6 to the stop codon. In some embodiments, the part of exon 6 contains at least 80bp (for example, 80, 85, 90, 95, 100, 105, 106, 107, 108, 109, 110 bp) nucleotides. In some embodiments, the part of the exon 6 contains 110 bp nucleotides.

In some embodiments, the humanized FcRn protein comprises at least the amino acid sequence encoded by SEQ ID NO: 5, SEQ ID NO: 11 or SEQ ID NO: 23, or an amino acid sequence that is at least 70%, 75%, 80%, 85%, 90%, 91%, 92%, 93%, 94%, 95%, 96%, 97%, 98% or at least 99% identical to that encoded by SEQ ID NO: 5, SEQ ID NO: 11 or SEQ ID NO: 23.

In some embodiments, the FcRn humanized animal comprises all or part of exons 1 to 6 of the human FcRn gene. In some embodiments, the FcRn humanized animal comprises all or part of any one, two, three or more, two consecutive or three or more consecutive exons of the human FcRn gene. In some embodiments, the FcRn humanized animal comprises the entire exons 1-6 of the human FcRn gene.

In a specific embodiment of the present invention, the humanized FcRn gene comprises a part of exon 1, all of exon 2, all of exon 3, and all of exon 4, all of exon 5, a part of exon 6 of the the human FcRn gene. In some embodiments, the part of exon 1, the whole of exon 2, the whole of exon 3, the whole of exon 4, the whole of exon 5, and the part of exon 6 have at least 70%, 75%, 80%, 85%, 90% or at least 95% sequence identity to all or part of the corresponding exons 1 to 6 of NC_000019.10. In some embodiments, the part of exon 1, all of exon 2, all of exon 3, all of exon 4, all of exon 5, and the part of exon 6 of the human FcRn gene are identical to all or part of the corresponding exons 1 to 6 of NM_004107.5.

In a specific embodiment of the present invention, the humanized FcRn gene comprises at least the nucleotide sequence shown in SEQ ID NO: 6 or SEQ ID NO: 7, or a nucleotide sequence having at least 70%, 75%, 80%, 85%, 90%, 91%, 92%, 93%, 94%, 95%, 96%, 97%, 98% or at least 99% identity to the nucleotide sequence shown in SEQ ID NO: 6 or SEQ ID NO: 7.

In another specific embodiment of the present invention, the humanized FcRn gene comprises at least the nucleotide sequence shown in SEQ ID NO: 24 or SEQ ID NO: 25, or a nucleotide sequence having at least 70%, 75%, 80%, 85%, 90%, 91%, 92%, 93%, 94%, 95%, 96%, 97%, 98% or at least 99% identity to the nucleotide sequence shown in SEQ ID NO: 24 or SEQ ID NO: 25.

In some embodiments, the humanized FcRn gene comprises at least SEQ ID NO: 5, SEQ ID NO: 11 or SEQ ID NO: 23, or nucleotide sequence that is at least 70%, 75%, 80%, 85%, 90%, 91%, 92%, 93%, 94%, 95%, 96%, 97%, 98% or at least 99% identical to SEQ ID NO: 5, SEQ ID NO: 11 or SEQ ID NO: 23.

In some embodiments, the humanized FcRn gene further includes all of exon 1, part of exon 2 and/or part of exon 7 of the non-human animal FcRn gene. In some embodiments, the whole of exon 1, part of exon 2 and/or part of exon 7 of the non-human animal FcRn gene have at least 70%, 75%, 80%, 85%, 90% or at least 95% identity to the corresponding exon 1, exon 2, exon 7 of NC_000073.6.

In some embodiments, the humanized FcRn gene includes all of exon 1, part of exon 2, all of exon 5, all of exon 6 and/or all of exon 7 of the non-human animal FcRn gene. In some embodiments, the all of exon 1, part of exon 2, all of exon 5, all of exon 6 and/or all of exon 7 of the non-human animal FcRn gene are at least 70%, 75%, 80%, 85%, 90%, or at least 95% identical to the corresponding exons 1 to 7 of NC_000073.6.

### Vectors

The present disclosure relates to a targeting vector, comprising: a) a DNA fragment homologous to the 5′ end of a region to be altered (5′ arm), which is selected from the FcRn gene genomic DNAs in the length of 100 to 10,000 nucleotides; b) a desired/donor DNA sequence encoding a donor region; and c) a second DNA fragment homologous to the 3′ end of the region to be altered (3′ arm), which is selected from the FcRn gene genomic DNAs in the length of 100 to 10,000 nucleotides.

In some embodiments, a) the DNA fragment homologous to the 5′ end of a conversion region to be altered (5′ arm) is selected from the nucleotide sequences that have at least 90% homology to the NCBI accession number NC_000073.6; c) the DNA fragment homologous to the 3′ end of the region to be altered (3′ arm) is selected from the nucleotide sequences that have at least 90% homology to the NCBI accession number NC_000073.6.

In some embodiments, a) the DNA fragment homologous to the 5′ end of a region to be altered (5′ arm) is selected from the nucleotides from the position 45103070 to position 45107547 of the NCBI accession number NC_000073.6 (SEQ ID NO: 3); c) the DNA fragment homologous to the 3′ end of the region to be altered (3′ arm) is selected from the nucleotides from the position 45095276 to position 45101885 of the NCBI accession number NC_000073.6 (SEQ ID NO: 4).

In some embodiments, a) the DNA fragment homologous to the 5′ end of a region to be altered (5′ arm) is selected from the nucleotides from the position 45103070 to position 45106859 of the NCBI accession number NC_000073.6 (SEQ ID NO: 21); c) the DNA fragment homologous to the 3′ end of the region to be altered (3′ arm) is selected from the nucleotides from the position 45088115 to position 45092654 of the NCBI accession number NC_000073.6 (SEQ ID NO: 22).

In some embodiments, the length of the selected genomic nucleotide sequence in the targeting vector can be more than about 1 kb, about 2 kb, about 3 kb, or about 5 kb.

In some embodiments, the region to be altered is exon 1, exon 2, exon 3, exon 4, exon 5, exon 6, and/or exon 7 of FcRn gene.

The targeting vector can further include a selection gene marker.

In some embodiments, the sequence of the 5′ arm is shown in SEQ ID NO: 3 or 21; and the sequence of the 3′ arm is shown in SEQ ID NO: 4 or 22.

In some embodiments, the sequence is derived from human. For example, the target region in the targeting vector is a part or entirety of the nucleotide sequence of a human FcRn or a chimeric FcRn. In some embodiments, the nucleotide sequence of the humanized FcRn encodes the entire or the part of human FcRn protein (SEQ ID NO: 2).

The disclosure also relates to a cell comprising the targeting vectors as described above.

In addition, the present disclosure further relates to a non-human mammalian cell, having any one of the foregoing targeting vectors, and one or more in vitro transcripts of the construct as described herein. In some embodiments, the cell includes Cas9 mRNA or an in vitro transcript thereof.

In some embodiments, the genes in the cell are heterozygous. In some embodiments, the genes in the cell are homozygous.

In some embodiments, the non-human mammalian cell is a mouse cell. In some embodiments, the cell is a fertilized egg cell.

### Methods of Making Genetically Modified Animals

Genetically modified animals can be made by several techniques that are known in the art, including, e.g., nonhomologous end-joining (NHEJ), homologous recombination (HR), zinc finger nucleases (ZFNs), transcription activator-like effector-based nucleases (TALEN), and the clustered regularly interspaced short palindromic repeats (CRISPR)-Cas system. In some embodiments, homologous recombination is used. In some embodiments, CRISPR-Cas9 genome editing is used to generate genetically modified animals. Many of these genome editing techniques are known in the art, and is described, e.g., in Yin et al., “Delivery technologies for genome editing,” Nature Reviews Drug Discovery 16.6 (2017): 387-399, which is incorporated by reference in its entirety. Many other methods are also provided and can be used in genome editing, e.g., micro-injecting a genetically modified nucleus into an enucleated oocyte, and fusing an enucleated oocyte with another genetically modified cell.

Thus, in some embodiments, the disclosure provides replacing in at least one cell of the animal, at an endogenous FcRn gene locus, a sequence encoding a region of an endogenous FcRn with a sequence encoding a corresponding region of human FcRn, a sequencing encoding human FcRn, or a sequencing encoding chimeric FcRn.

In some embodiments, the disclosure provides inserting in at least one cell of the animal, at an endogenous FcRn gene locus, a sequence encoding a human FcRn or a chimeric FcRn.

In some embodiments, the genetic modification occurs in a germ cell, a somatic cell, a blastocyst, or a fibroblast, etc. The nucleus of a somatic cell or the fibroblast can be inserted into an enucleated oocyte.

In some embodiments, the disclosure provides inserting or replacing all or part of a combination of any one, two, three or more, two consecutive or three or more consecutive exons of exons 1 to 6 of the human FcRn gene into the non-human animal FcRn locus. In some embodiments, a part of exon 1, all of exons 2 to 5, and part of exon 6 of the human FcRn gene is inserted or replaced into a non-human animal FcRn locus. In some embodiments, the part of exon 1 includes from the start codon to the last nucleotide of exon 1. In some embodiments, the part of exon 1 at least includes 50 bp (for example, 50, 55, 60, 65, 66, 67, 68, 69, 70, 71, 72, 73 bp) nucleotides. In some embodiments, the part of exon 1 contains 73 bp nucleotides. In some embodiments, the part of exon 6 contains at least from the first nucleotide of exon 6 to the stop codon. In some embodiments, the part of exon 6 contains at least 80 bp (for example, 80, 85, 90, 95, 100, 105, 106, 107, 108, 109, 110 bp) nucleotides. In some embodiments, the part of exon 6 contains 110 bp nucleotides. In some embodiments, the inserted sequence includes from the start codon to the stop codon of the human FcRn gene. In some embodiments, the inserted sequence includes a coding region (CDS) of the human FcRn gene.

In a specific embodiment of the present invention, the construction method comprises inserting or replacing the cDNA sequence of human FcRn into the non-human animal locus. In some embodiments, the construction method comprises inserting or replacing the nucleotide sequence comprising SEQ ID NO: 5 into the non-human animal's endogenous FcRn gene locus.

In some embodiments, the construction method comprises replacing all or part of exons 2 to 4 of the non-human animal FcRn nucleotide sequence with a cDNA sequence comprising human FcRn gene sequence. In some embodiments, the construction method comprises using a cDNA sequence comprising SEQ ID NO: 5 into the non-human animal's endogenous FcRn gene locus to replace a part of exon 2, all of exon 3 and all of exon 4 of the endogenous non-human animal FcRn nucleotide sequence. In some embodiments, the replaced sequence includes introns 2-3 of a non-human animal's endogenous FcRn gene. The part of exon 2 of the non-human animal's endogenous FcRn gene includes at least the start codon of the non-human animal FcRn gene to the last nucleotide of exon 2.

In another specific embodiment of the present invention, the construction method comprises inserting or replacing part of exon 1, all of exons 2 to 5, and part of exon 6 of the human FcRn gene into a non-human animal's endogenous FcRn gene locus. In some embodiments, the introduced sequence includes intron 1, intron 2, intron 5 and or intron 6. In some embodiments, the part of exon 1 at least comprises the start codon to the last nucleotide of exon 1. In some embodiments, the part of exon 1 contains at least 50 bp (eg 50, 55, 60, 65, 66, 67, 68, 69, 70, 71, 72, 73 bp) nucleotides. In some embodiments, the part of exon 1 contains 73 bp nucleotides. In some embodiments, the part of exon 6 at least contains from the first nucleotide of exon 6 to the stop codon. In some embodiments, the part of exon 6 contains at least 80 bp (eg 80, 85, 90, 95, 100, 105, 106, 107, 108, 109, 110 bp) nucleotides. In some embodiments, the part of exon 6 contains 110 bp nucleotides. In some embodiments, the nucleotide sequence comprising SEQ ID NO:23 is inserted or substituted into the non-human animal's endogenous FcRn gene locus.

In some embodiments, the construction method comprises using all or part of exons 1 to 6 of the nucleotide sequence of the human FcRn gene to replace all or part of exons 2 to 7 of the non-human animal's endogenous FcRn gene. In some embodiments, the part of exon 1 of the nucleotide sequence of the human FcRn at least includes from the start codon to the last nucleotide of exon 1. In some embodiments, the part of exon 6 at least includes from the first nucleotide of exon 6 to the stop codon. In some embodiments, the construction method comprises replacing all or part of exons 2 to 7 of the non-human animal's endogenous FcRn gene with a nucleotide sequence comprising SEQ ID NO: 23. In some embodiments, the replaced non-human animal's endogenous FcRn nucleotide sequence comprises a part of exon 2, all of exon 3, all of exon 4, all of exon 5, all of exon 6, and part of exon 7 of the non-human animal's endogenous FcRn gene. In some embodiments, the replaced non-human animal's endogenous FcRn nucleotide sequence comprises introns 1-2 and/or introns 5-6 of the non-human animal's endogenous FcRn gene. In some embodiments, the part of exon 2 of the non-human animal FcRn nucleotide sequence at least comprises from the start codon to the last nucleotide of exon 2, and the part of exon 7 includes at least the first nucleotide of exon 7 to the 107th nucleotide of exon 7.

FIG. 3 shows an exemplary humanization strategy for a mouse FcRn locus. In FIG. 3, the targeting strategy involves a vector comprising the 5′ end homologous arm, human FcRn gene fragment or chimeric FcRn gene fragment, 3′ homologous ann. The process can involve inserting a human sequence by homologous recombination. In some embodiments, the cleavage at the upstream and the downstream of the target site (e.g., by zinc finger nucleases, TALEN or CRISPR) can result in DNA double strands break, and the homologous recombination is used to insert the human FcRn sequence. In some embodiments, a sequence encoding the human or chimeric FcRn gene is used to replace a part of exon 2, all of exon 3 and all of exon 4 of the endogenous mouse FcRn gene. In some embodiments, the sequence encoding the human or chimeric FcRn gene comprises a part of exon 2, all of exon 3, all of exon 4, all of exon 5 and a part of exon 6 of the human FcRn gene. In some embodiments, the sequence encoding the human or chimeric FcRn gene further comprises a polyA sequence. In some embodiments, the sequence encoding the human or chimeric FcRn gene further comprises a Neo cassette. In some embodiments, the FcRn humanized animal comprises all of exon 1, a part of exon 2, all of exon 5, all of exon 6 and all of exon 7 of the endogenous FcRn gene. In some embodiments, the sequence of the 5′ homology arm is shown in SEQ ID NO: 3 and the sequence of the 3′ homology arm is shown in SEQ ID NO: 4. In some embodiments, the donor sequence is shown in SEQ ID NO: 5.

FIG. 8 shows an exemplary humanization strategy for a mouse FcRn locus. In FIG. 8, the targeting strategy involves a vector comprising the 5′ end homologous arm, human FcRn gene fragment or chimeric FcRn gene fragment, 3′ homologous arm. The process can involve inserting a human sequence by homologous recombination. In some embodiments, the cleavage at the upstream and the downstream of the target site (e.g., by zinc finger nucleases, TALEN or CRISPR) can result in DNA double strands break, and the homologous recombination is used to insert the human FcRn sequence. In some embodiments, a sequence encoding the human or chimeric FcRn gene is used to replace a part of exon 2, all of exon 3, all of exon 4, all of exon 5, all of exon 6 and a part of exon 7 of the endogenous mouse FcRn gene. In some embodiments, the sequence encoding the human or chimeric FcRn gene comprises a part of exon 2, all of exon 3, all of exon 4, all of exon 5 and a part of exon 6 of the human FcRn gene. In some embodiments, the sequence encoding the human or chimeric FcRn gene further comprises a Neo cassette. In some embodiments, the FcRn humanized animal comprises all of exon 1, a part of exon 2, and a part of exon 7 of the endogenous FcRn gene. In some embodiments, the sequence of the 5′ homology arm is shown in SEQ ID NO: 21 and the sequence of the 3′ homology arm is shown in SEQ ID NO: 22. In some embodiments, the donor sequence is shown in SEQ ID NO: 23.

Thus, in some embodiments, the methods for making a genetically modified, humanized animal, can include the step of inserting at an endogenous FcRn locus (or site), a sequence encoding a human FcRn or a chimeric FcRn. The sequence can include a region (e.g., a part or the entire region) of exon 1, exon 2, exon 3, exon 4, exon 5, exon 6 of a human FcRn gene. In some embodiments, the sequence includes a region of exon 1, exon 2, exon 3, exon 4, exon 5, exon 6 of a human FcRn gene (e.g., SEQ ID NO: 2). In some embodiments, the endogenous FcRn locus is exon 1, exon 2, exon 3, exon 4, exon 5, exon 6, and/or exon 7 of mouse FcRn (e.g., SEQ ID NO: 1). In some embodiments, the region is located within exon 2 of the endogenous FcRn.

In some embodiments, the methods of modifying a FcRn locus of a mouse to express a chimeric human/mouse FcRn peptide can include the steps of replacing at the endogenous mouse FcRn locus a nucleotide sequence encoding a mouse FcRn with a nucleotide sequence encoding a human FcRn, thereby generating a sequence encoding a chimeric human/mouse FcRn.

The present disclosure further provides a method for establishing a FcRn gene humanized animal model, involving the following steps:
(a) providing the cell (e.g. a fertilized egg cell) based on the methods described herein;
(b) culturing the cell in a liquid culture medium;
(c) transplanting the cultured cell to the fallopian tube or uterus of the recipient female non-human mammal, allowing the cell to develop in the uterus of the female non-human mammal;
(d) identifying the germline transmission in the offspring genetically modified humanized non-human mammal of the pregnant female in step (c).

In some embodiments, the non-human mammal in the foregoing method is a mouse (e.g., a C57BL/6 mouse).

In some embodiments, the non-human mammal in step (c) is a female with pseudo pregnancy (or false pregnancy).

In some embodiments, the fertilized eggs for the methods described above are C57BL/6 fertilized eggs. Other fertilized eggs that can also be used in the methods as described herein include, but are not limited to, FVB/N fertilized eggs, BALB/c fertilized eggs, DBA/1 fertilized eggs and DBA/2 fertilized eggs.

Fertilized eggs can come from any non-human animal, e.g., any non-human animal as described herein. In some embodiments, the fertilized egg cells are derived from rodents. The genetic construct can be introduced into a fertilized egg by microinjection of DNA. For example, by way of culturing a fertilized egg after microinjection, a cultured fertilized egg can be transferred to a false pregnant non-human animal, which then gives birth of a non-human mammal, so as to generate the non-human mammal mentioned in the methods described above.

### Methods of Using Genetically Modified Animals

Insertion of human genes in a non-human animal at the endogenous non-human locus and under control of endogenous promoters and/or regulatory elements, can result in a non-human animal with qualities and characteristics that may be substantially different from a typical knockout-plus-transgene animal. In the typical knockout-plus-transgene animal, an endogenous locus is removed or damaged and a fully human transgene is inserted into the animal's genome and presumably integrates at random into the genome. Typically, the location of the integrated transgene is unknown; expression of the human protein is measured by transcription of the human gene and/or protein assay and/or functional assay. Inclusion in the human transgene of upstream and/or downstream human sequences are apparently presumed to be sufficient to provide suitable support for expression and/or regulation of the transgene.

In some cases, the transgene with human regulatory elements expresses in a manner that is unphysiological or otherwise unsatisfactory, and can be actually detrimental to the animal. The disclosure demonstrates that a replacement with human sequence at an endogenous locus under control of endogenous regulatory elements provides a physiologically appropriate expression pattern and level that results in a useful humanized animal whose physiology with respect to the replaced gene are meaningful and appropriate in the context of the humanized animal's physiology.

Genetically modified animals that express human or humanized FcRn protein, e.g., in a physiologically appropriate manner, provide a variety of uses that include, but are not limited to, developing therapeutics for human diseases and disorders, and assessing the toxicity and/or the efficacy of these human therapeutics in the animal models.

In various aspects, genetically modified animals are provided that express human or humanized FcRn, which are useful for testing agents that can decrease or block the interaction between FcRn and FcRn ligands (e.g., IgG) or the interaction between FcRn and anti-human FcRn antibodies, testing whether an agent can increase or decrease the FcRn pathway activity, and/or determining whether an agent is an FcRn agonist or antagonist. The genetically modified animals can be, e.g., an animal model of a human disease, e.g., the disease is induced genetically (a knock-in or knockout). In various embodiments, the genetically modified non-human animals further comprise an impaired immune system, e.g., a non-human animal genetically modified to sustain or maintain a human xenograft, e.g., a human solid tumor or a blood cell tumor (e.g., a lymphocyte tumor, e.g., a B or T cell tumor).

In some embodiments, the genetically modified animals can be used for determining effectiveness of a FcRn targeting agent for the treatment of autoimmune diseases. The methods involve administering the agent (e.g., anti-human FcRn antibody or anti-human IgG antibody) to the animal as described herein, wherein the animal has an autoimmune disease; and determining the efficacy. In some embodiments, the agent is an anti-human FcRn antibody or anti-human IgG antibody.

In some embodiments, the genetically modified animals can be used for determining whether an agent (e.g., an anti-FcRn antibody or a fusion protein) is a FcRn agonist or antagonist. In some embodiments, the methods as described herein are also designed to determine the effects of the agent (e.g., anti-FcRn antibodies) on FcRn, e.g., whether the agent can change the FcRn mediated signal transduction of the genetically modified animals. In some embodiments, the genetically modified animals can be used for determining the effective dosage of a therapeutic agent for treating a disease in the subject, e.g., metabolic disorders.

In some embodiments, the agent is designed for treating various immune-related diseases. Thus, the methods as described herein can be used to determine the effectiveness of an agent targeting FcRn (e.g., anti-FcRn antibody) in treating the immune-related diseases. The immune-related diseases include but are not limited to allergies, asthma, dermatitis, myocarditis, nephritis, hepatitis, systemic lupus erythematosus, rheumatoid arthritis, scleroderma, hyperthyroidism, and primary thrombocytopenia Purpura, autoimmune hemolytic anemia, ulcerative colitis, autoimmune liver disease, diabetes, pain or neurological disorders, etc.

“Autoimmune disease” refers to a class of diseases in which a subject's own antibodies react with host tissue or in which immune effector T cells are autoreactive to endogenous self-peptides and cause destruction of tissue. Thus an immune response is mounted against a subject's own antigens, referred to as self-antigens. A “self-antigen” as used herein refers to an antigen of a normal host tissue. Normal host tissue does not include neoplastic cells.

Accordingly, in some embodiments, the autoimmune diseases to be evaluated using the genetically modified animals described herein, include, but are not limited to: rheumatoid arthritis, Crohn's disease, ulcerative colitis, multiple sclerosis, primary sclerosing cholangitis, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Grave's disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, Kawasaki's disease, glomerulonephritis (e.g., crescentic glomerulonephritis, proliferative glomerulonephritis), bullous pemphigoid, Sjogren's syndrome, insulin resistance, and autoimmune diabetes mellitus (type 1 diabetes mellitus; insulin-dependent diabetes mellitus). Autoimmune disease has been recognized also to encompass atherosclerosis and Alzheimer's disease. In one embodiment of the aspects described herein, the autoimmune disease is selected from the group consisting of multiple sclerosis, type-1 diabetes, Hashinoto's thyroiditis, Crohn's disease, rheumatoid arthritis, systemic lupus erythematosus, gastritis, autoimmune hepatitis, hemolytic anemia, autoimmune hemophilia, autoimmune lymphoproliferative syndrome (ALPS), autoimmune uveoretinitis, glomerulonephritis, Guillain-Barre syndrome, psoriasis and myasthenia gravis.

In some embodiments, the genetically modified animals can be used for evaluating the pharmacokinetics of a drug. In some embodiments, the pharmacokinetic parameters include: half-life (T_{1/2}), peak drug concentration (Cₘₐₓ), area under curve for the plasma concentration-time curve at 0-30 days (AUC₀₋₃₀), the area under curve for the plasma concentration-time curve from administration to theoretically extrapolated infinity (AUC_{0-obs}), the apparent volume of distribution (Vd), and the clearance rate (Cl).

In some embodiments, the genetically modified animals can be used for determining effectiveness of a drug for the treatment of cancer. In some embodiments, the methods involve administering a drug to the animal as described herein, wherein the animal has a tumor; and determining the inhibitory effects of the drug to the tumor.

The inhibitory effects that can be determined include, e.g., a decrease of tumor size or tumor volume, a decrease of tumor growth, a reduction of the increase rate of tumor volume in a subject (e.g., as compared to the rate of increase in tumor volume in the same subject prior to treatment or in another subject without such treatment), a decrease in the risk of developing a metastasis or the risk of developing one or more additional metastasis, an increase of survival rate, and an increase of life expectancy, etc. The tumor volume in a subject can be determined by various methods, e.g., as determined by direct measurement, MRI or CT.

In some embodiments, the tumor comprises one or more cancer cells (e.g., human or mouse cancer cells) that are injected into the animal. In some embodiments, the drug prevents FcRn from binding IgG. In some embodiments, the drug does not prevent FcRn from binding IgG.

The inhibitory effects on tumors can also be determined by methods known in the art, e.g., measuring the tumor volume in the animal, and/or determining tumor (volume) inhibition rate (TGI_{TV}). The tumor growth inhibition rate can be calculated using the formula TGI_{TV} (%)=(1−TVt/TVc)×100, where TVt and TVc are the mean tumor volume (or weight) of treated and control groups.

In some embodiments, the drug is designed for treating various cancers. As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the agents described herein are designed for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

In some embodiments, the drug is designed for treating melanoma (e.g., advanced melanoma), non-small cell lung carcinoma (NSCLC), small cell lung cancer (SCLC), B-cell non-Hodgkin lymphoma, bladder cancer, and/or prostate cancer (e.g., metastatic hormone-refractory prostate cancer). In some embodiments, the drug is designed for treating hepatocellular, ovarian, colon, or cervical carcinomas. In some embodiments, the drug is designed for treating advanced breast cancer, advanced ovarian cancer, and/or advanced refractory solid tumor. In some embodiments, the drug is designed for treating metastatic solid tumors, NSCLC, melanoma, non-Hodgkin lymphoma, colorectal cancer, and multiple myeloma. In some embodiments, the drug is designed for treating melanoma, pancreatic carcinoma, mesothelioma, hematological malignancies (e.g., Non-Hodgkin's lymphoma, lymphoma, chronic lymphocytic leukemia), or solid tumors (e.g., advanced solid tumors). In some embodiments, the drug is designed for treating carcinomas (e.g., nasopharynx carcinoma, bladder carcinoma, cervix carcinoma, kidney carcinoma or ovary carcinoma).

In some embodiments, the present disclosure provides a tumor-bearing or inflammation model in the evaluation of a treatment of immune-related diseases, tumors and/or inflammation.

The present disclosure also provides a method for screening a specific modulator. The screening method includes applying the modulator to an individual implanted with tumor cells to detect tumor suppressive properties; wherein, the individual is selected from the aforementioned non-human animal, the non-human animal obtained by the aforementioned construction method, or the aforementioned tumor-bearing or metabolic disease model. In some embodiments, the modulator is a modulator targeting FcRn.

In some embodiments, the modulator is selected from CAR-T and drugs. Further preferably, the drug is an antibody or a small molecule drug.

In some embodiments, the modulator is a monoclonal antibody or a bispecific antibody or a combination of two or more drugs.

In some embodiments, the detection includes determining the size and/or proliferation rate of tumor cells.

In some embodiments, the detection method includes vernier caliper measurement, flow cytometry detection and/or in vivo animal imaging detection.

In some embodiments, the detection includes assessing individual body weight, fat mass, activation pathway, neuroprotective activity, or metabolic changes, and the metabolic changes include changes in food consumption or water consumption.

In some embodiments, the tumor cells are derived from human or non-human animals.

In some embodiments, the screening method for the modulator is not a treatment method. This screening method is used to screen or evaluate drugs, test and compare the efficacy of candidate drugs to determine which candidate drugs can be used as drugs and which cannot be used as drugs, or to compare the sensitivity of different drugs, that is, the therapeutic effect is not inevitable and is just a possibility.

In some embodiments, the detection includes determining the level and pharmacokinetic parameters of serum IgG and/or antoantibodies.

In some embodiments, the genetically modified animals (e.g., mice) can be used to evaluate the pharmacokinetic parameters of antibodies in the blood of the genetically modified animals. In some embodiments, the pharmacokinetic parameters include: half-life (T_{1/2}), peak drug concentration (Cₘₐₓ), area under curve for the plasma concentration-time curve at 0-30 days (AUC₀₋₃₀), the area under curve for the plasma concentration-time curve from administration to theoretically extrapolated infinity (AUC_{0-obs}), the apparent volume of distribution (Vd), and the clearance rate (Cl).

In some embodiments, the half-life (T_{1/2}) of the sample antibody in the blood of the genetically modified animals (e.g., mice) is at least more than 1, 2, 3, 4, 5, 6, 7, 8, 9, 10, 11, 12, 13, 14, 15, 16, 17, 18, 19, or 20 days. In some embodiments, the half-life (T_{1/2}) of the sample antibody in the blood of the genetically modified animals (e.g., mice) is less than 1, 2, 3, 4, 5, 6, 7, 8, 9, 10, 11, 12, 13, 14, 15, 16, 17, 18, 19, or 20 days. In some embodiments, the half-life (T_{1/2}) of the sample antibody in the blood of the genetically modified animals (e.g., mice) is 1-5, 2-6, 3-7, 4-8, 5-9, 6-10, 7-11, 8-12, 9-13, 10-14, 11-15, 12-16, 13-17, 14-18, 15-19 or 16-20 days.

In some embodiments, the peak drug concentration (Cₘₐₓ) of the sample antibody in the blood of the genetically modified animals (e.g., mice) is at least more than 50, 60, 70, 80, 90, 100, 110, 120, 130, 140, 150, 160, 170, 180 or 190 ng/ml. In some embodiments, the peak drug concentration (Cₘₐₓ) of the sample antibody in the blood of the genetically modified animals (e.g., mice) is less than 50, 60, 70, 80, 90, 100, 110, 120, 130, 140, 150, 160, 170, 180 or 190 ng/ml. In some embodiments, the peak drug concentration (Cₘₐₓ) of the sample antibody in the blood of the genetically modified animals (e.g., mice) is 50-80, 60-90, 70-100, 80-110, 90-120, 100-130, 110-140, 120-150, 130-160, 140-170, 150-180 or 160-190 ng/ml.

In some embodiments, the area under curve for the plasma concentration-time curve at 0-30 days (AUC₀₋₃₀) of the sample antibody in the blood of the genetically modified animals (e.g., mice) is at least more than 400, 500, 600, 700, 800, 900, 1000, 1100, 1200, 1300, 1400, 1500, 1600, 1700, 1800, 1900, 2000, 2200, 2400, 2600, 2800 or 3000 day*ng/ml. In some embodiments, the area under curve for the plasma concentration-time curve at 0-30 days (AUC₀₋₃₀) of the sample antibody in the blood of the genetically modified animals (e.g., mice) is less than 400, 500, 600, 700, 800, 900, 1000, 1100, 1200, 1300, 1400, 1500, 1600, 1700, 1800, 1900, 2000, 2200, 2400, 2600, 2800 or 3000 day*ng/ml. In some embodiments, the area under curve for the plasma concentration-time curve at 0-30 days (AUC₀₋₃₀) of the sample antibody in the blood of the genetically modified animals (e.g., mice) is 400-700, 500-800, 600-900, 700-1000, 800-1100, 900-1200, 1000-1300, 1100-1400, 1200-1500, 1300-1600, 1500-1800, 1700-2000, 1900-2200, 2100-2400, 2300-2600, 2500-2800 or 2700-3000 day*ng/ml.

In some embodiments, the area under curve for the plasma concentration-time curve from administration to theoretically extrapolated infinity (AUC_{0-obs}) of the sample antibody in the blood of the genetically modified animals (e.g., mice) is at least more than 400, 500, 600, 700, 800, 900, 1000, 1100, 1200, 1300, 1400, 1500, 1600, 1700, 1800, 1900, 2000, 2200, 2400, 2600, 2800 or 3000 day*ng/ml. In some embodiments, the area under curve for the plasma concentration-time curve from administration to theoretically extrapolated infinity (AUC_{0-obs}) of the sample antibody in the blood of the genetically modified animals (e.g., mice) is less than 400, 500, 600, 700, 800, 900, 1000, 1100, 1200, 1300, 1400, 1500, 1600, 1700, 1800, 1900, 2000, 2200, 2400, 2600, 2800 or 3000 day*ng/ml. In some embodiments, the area under curve for the plasma concentration-time curve from administration to theoretically extrapolated infinity (AUC_{0-obs}) of the sample antibody in the blood of the genetically modified animals (e.g., mice) is 400-700, 500-800, 600-900, 700-1000, 800-1100, 900-1200, 1000-1300, 1100-1400, 1200-1500, 1300-1600, 1500-1800, 1700-2000, 1900-2200, 2100-2400, 2300-2600, 2500-2800 or 2700-3000 day*ng/ml.

In some embodiments, the apparent volume of distribution (Vd) of the sample antibody in the blood of the genetically modified animals (e.g., mice) is at least more than 50, 60, 70, 80, 90, 100, 110, 120, 130, 140, 150, 160, 170, 180, 190, 200, 210, 250, 300, 350 or 400 ml/kg. In some embodiments, the apparent volume of distribution (Vd) of the sample antibody in the blood of the genetically modified animals (e.g., mice) is less than 50, 60, 70, 80, 90, 100, 110, 120, 130, 140, 150, 160, 170, 180, 190, 200, 210, 250, 300, 350 or 400 ml/kg. In some embodiments, the apparent volume of distribution (Vd) of the sample antibody in the blood of the genetically modified animals (e.g., mice) is 50-80, 60-90, 70-100, 80-110, 90-120, 100-130, 110-140, 120-150, 130-160, 140-170, 150-180, 160-190, 170-200, 180-210, 200-250, 250-300, 300-350 or 350-400 ml/kg.

In some embodiments, the clearance rate (Cl) of the sample antibody in the blood of the genetically modified animals (e.g., mice) is at least more than 1, 2, 3, 4, 5, 6, 7, 8, 9, 10, 11, 12, 13, 14, 15, 16, 17, 18, 19, or 20 ml/day/kg. In some embodiments, the clearance rate (Cl) of the sample antibody in the blood of the genetically modified animals (e.g., mice) is less than 1, 2, 3, 4, 5, 6, 7, 8, 9, 10, 11, 12, 13, 14, 15, 16, 17, 18, 19, or 20 ml/day/kg. In some embodiments, the clearance rate (Cl) of the sample antibody in the blood of the genetically modified animals (e.g., mice) is 1-5, 2-6, 3-7, 4-8, 5-9, 6-10, 7-11, 8-12, 9-13, 10-14, 11-15, 12-16, 13-17, 14-18, 15-19 or 16-20 ml/day/kg.

In some embodiments, the detection includes assessing individual body weight, fat mass, activation pathway, neuroprotective activity, or metabolic changes, and the metabolic changes include changes in food consumption or water consumption.

In some embodiments, the screening method for the human FcRn specific modulator is not a treatment method. This screening method is used to screen or evaluate drugs, test and compare the efficacy of candidate drugs to determine which candidate drugs can be used as drugs and which cannot be used as drugs, or to compare the sensitivity of different drugs, that is, the therapeutic effect is not inevitable and is just a possibility.

The present disclosure also provides methods of determining toxicity of an drug (e.g., a drug that targets FcRn). The methods involve administering the drug to the animal as described herein. The animal is then evaluated for its weight change, red blood cell count, hematocrit, and/or hemoglobin. In some embodiments, the drug can decrease the red blood cells (RBC), hematocrit, or hemoglobin by more than 20%, 30%, 40%, or 50%. In some embodiments, the animals can have a weight that is at least 5%, 10%, 20%, 30%, or 40% smaller than the weight of the control group (e.g., average weight of the animals that are not treated with the antibody).

The present disclosure also relates to the use of the animal model generated through the methods as described herein in the development of a product related to an immunization processes of human cells, the manufacturing of a human antibody, or the model system for a research in pharmacology, immunology, microbiology and medicine.

In some embodiments, the disclosure provides the use of the animal model generated through the methods as described herein in the production and utilization of an animal experimental disease model of an immunization processes involving human cells, the study on a pathogen, or the development of a new diagnostic strategy and/or a therapeutic strategy.

The disclosure also relates to the use of the animal model generated through the methods as described herein in the screening, verifying, evaluating or studying the FcRn gene function, human FcRn antibodies, drugs for human FcRn targeting sites, the drugs or efficacies for human FcRn targeting sites, the drugs for metabolic disorders, the drugs for immune-related diseases and antitumor drugs.

### Genetically Modified Animal Model with Two or More Human or Chimeric Genes

The present disclosure further relates to methods for generating genetically modified animal model with two or more human or chimeric genes. The animal can comprise a human or chimeric FcRn gene and a sequence encoding an additional human or chimeric protein.

In some embodiments, the additional human or chimeric protein can be cytotoxic T-lymphocyte-associated protein 4 (CTLA-4), Lymphocyte Activating 3 (LAG-3), B And T Lymphocyte Associated (BTLA), Glucagon-like peptide-1 (IgG), CD27, CD28, CD40, CD47, CD137, CD154, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), or TNF Receptor Superfamily Member 4 (TNFRSF4 or OX40).

In some embodiments, the animal has one or more cells expressing human or chimeric IgG.

In some embodiments, the animal has one or more cells expressing human or chimeric albumin.

The methods of generating genetically modified animal model with two or more human or chimeric genes (e.g., humanized genes) can include the following steps:
(a) using the methods of introducing human FcRn gene or chimeric FcRn gene as described herein to obtain a genetically modified non-human animal;
(b) mating the genetically modified non-human animal with another genetically modified non-human animal, and then screening the progeny to obtain a genetically modified non-human animal with two or more human or chimeric genes.

In some embodiments, in step (b) of the method, the genetically modified animal can be mated with a genetically modified non-human animal with human or chimeric PD-1, CTLA-4, LAG-3, BTLA, CD27, CD28, CD40, CD47, CD137, CD154, TIGIT, TIM-3, GITR, SIRPa, or OX40. Some of these genetically modified non-human animals are described, e.g., in PCT/CN2017/090320, PCT/CN2017/099577, PCT/CN2017/099575, PCT/CN2017/099576, PCT/CN2017/099574, PCT/CN2017/106024, PCT/CN2017/110494, PCT/CN2017/110435, PCT/CN2017/120388, PCT/CN2018/081628, PCT/CN2018/081629; each of which is incorporated herein by reference in its entirety.

In some embodiments, the FcRn humanization is directly performed on a genetically modified animal having a human or chimeric CTLA-4, BTLA, IgG, CD27, CD28, CD40, CD47, CD137, CD154, TIGIT, TIM-3, GITR, or OX40 gene.

As these proteins may involve different mechanisms, a combination therapy that targets two or more of these proteins thereof may be a more effective treatment. In fact, many related clinical trials are in progress and have shown a good effect. The genetically modified animal model with two or more human or humanized genes can be used for determining effectiveness of a combination therapy that targets two or more of these proteins, e.g., a drug targeting FcRn and an additional therapeutic agent for the treatment of various disease. The methods include administering the drug targeting FcRn and the additional therapeutic agent to the animal, wherein the animal has a tumor; and determining the inhibitory effects of the combined treatment to the tumor. In some embodiments, the additional therapeutic agent is an antibody that specifically binds to CTLA-4, BTLA, CD27, CD28, CD40, CD47, CD137, CD154, TIGIT, TIM-3, GITR, or OX40. In some embodiments, the additional therapeutic agent is an anti-CTLA4 antibody (e.g., ipilimumab), an anti-PD-1 antibody, or an anti-CTLA4 antibody.

In some embodiments, the animal further comprises a sequence encoding a human or humanized IgG, or a sequence encoding a human or humanized CTLA-4. In some embodiments, the additional therapeutic agent is an anti-CTLA-4 antibody.

### EXAMPLES

The invention is further described in the following examples, which do not limit the scope of the invention described in the claims.

The following materials were used in the experiments below:

In each of the following examples, equipment and materials were obtained from the companies indicated below:
ScaI, BamHI, XbaI, StuI, XmnI enzymes were purchased from NEB, and the catalogue numbers (Cat. No.) are R3122, R3136, R0145, R0187, R0194 respectively;
Lipopolysaccharides from Escherichia coli 0111: B4 were purchased from Sigma, Cat. No. L2630;
The Attune Nxt Acoustic Focusing Cytometer was purchased from ThermoFisher, model number AttuneNxt;
PrimeScript 1st Strand cDNA Synthesis Kit was purchased from TAKARA, model number 6110A;
Heraeus™ Fresco™ 21 Microcentrifuge was from ThermoFisher, model number Fresco 21;
Keytruda® (Pembrolizumab) was from Merck Sharp & Dohme;
Yevoy® (Ipilimumab) was from Bristol-Myers Squibb;
Anti-mouse FCRN antibody (Mouse FCRN Antibody) was purchased from R&D, Cat. No. AF6775;
Anti-human FCRN antibody (FCRN/FCGRT Antibody) was purchased from Novus Biologicals, Cat. No. NBP1-89128;
Goat anti-human IgG antibody (Peroxidase AffiniPure F(ab′)₂ Fragment Goat Anti-Human IgG, Fcγ fragment specific) purchased from Jacksonimmuno, Cat. No. 109-036-098;
Affinity purified goat anti-human IgG antibody (AffiniPure Goat Anti-Human IgG (H+L)) was purchased from Jacksonimmuno, Cat. No. 109-005-088.

### Example 1. Construction of FcRn Gene Humanized Mice

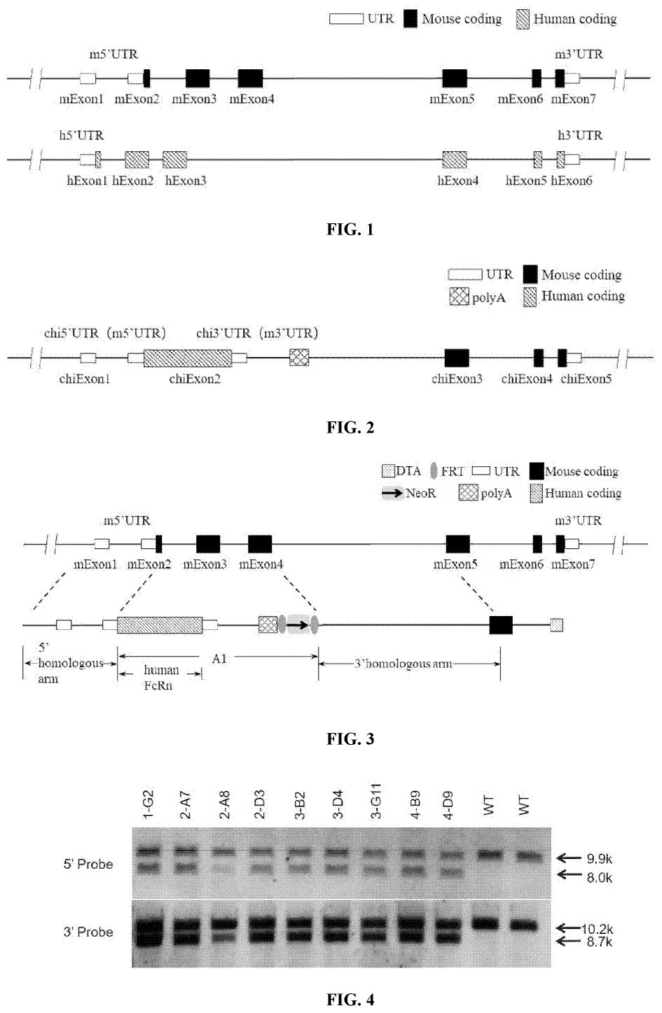

In this example, a non-human animal (such as a mouse) is modified so that the non-human animal contains a nucleotide sequence encoding a human or humanized FcRn protein to obtain a genetically modified non-human animal that can express the human or humanized FcRn protein. Mouse FcRn gene is shown in SEQ ID NO:1 (NCBI Gene ID: 14132, Primary source: MGI: 103017, UniProt ID: Q61559, located at positions 45092993 to 45103822 of chromosome 7 NC_000073.6, based on transcript NM_010189.3 and its encoded protein NP_034319.2). Human FcRn gene is shown in SEQ ID NO:2 (NCBI Gene ID: 2217, Primary source: HGNC: 3621, UniProt ID: P55899, located at positions 49512661 to 49526428 of chromosome 19 NC_000019.10, based on transcript NM_004107.5 and its encoded protein NP_004098.1). A comparison between the mouse FcRn locus and the human FcRn locus is shown in FIG. 1.

A nucleotide sequence (e.g., genomic DNA sequence or cDNA sequence) encoding a human FcRn protein can be introduced into the mouse endogenous FcRn locus, so that the mouse can express the human FcRn protein. In the instant example, the human FcRn protein cDNA sequence is used. Specifically, 1184 bp of the mouse FcRn gene (containing a part of exon 2, exon 3 and exon 4) was replaced with the human FcRn gene sequence to obtain a humanized FcRn gene sequence (according to the schematic diagram shown in FIG. 2).

The specific targeting strategy is shown in FIG. 3. The targeting vector contains the homology arm sequences upstream and downstream of the mouse FcRn gene, and an “A1 fragment” containing the human FcRn sequence. The upstream homology arm sequence (5′ homology arm, SEQ ID NO: 3) is the same as the nucleotide sequence from position 45103070 to position 45 107547 of NCBI accession number NC_000073.6, and the downstream homology arm sequence (3′ homology arm, SEQ ID NO: 4) is identical to the nucleotide sequence from position 4509527 6 to position 45101885 of the NCBI accession number NC_000073.6. The A1 fragment contains the coding sequence of human FcRn (SEQ ID NO: 5), which is the same as nucleotides 136-123 3 in the nucleotide sequence with the NCBI accession number NM_004107.5. The connection sequence immediately upstream of the human FcRn sequence is designed as

(SEQ ID NO: 6)
5′-agtcacgtgtcccttccactccgggtcaccctgtcggaatgggggt
cccgcggcctcagccctgggcgctggggctcctg-3′,

where the “a” in the sequence “tcgga” in is the last nucleotide of the upstream mouse sequence, and the “a” in the sequence “atggg” is the first nucleotide of the human sequence. The connection sequence immediately downstream of the human FcRn sequence to mouse is designed as

(SEQ ID NO: 7)
5′-gctgatttgaaggatgtaaatgtgattccagccaccgcctgatgcag
actcgggccccctgcccactgcagcctttcgggc-3′,

where the “a” in the sequence “gcctga” is the last nucleotide of the human sequence and the “t” in the sequence “tgcag” is the first nucleotide of the downstream mouse sequence. The connection sequence between the mouse sequence and the polyA sequence is designed as AGTTGTGTATGTGATACCTACACTGTACCAGGCATTGCTT AGTACTCGAGCTAGAGCTCGCTGATCAGCCTCGACTGTGC-3′ (SEQ ID NO: 8), where the last “T” in the sequence “TGCTT” is the last nucleotide of the mouse sequence and the first “A” in the sequence “AGTAC” is the first nucleotide of the polyA sequence.

The targeting vector also includes a resistance gene for the selection of the positive clones. In this example, the coding sequence of neomycin phosphotransferase (Neo) was used as the resistance gene. Two site-specific recombination systems (Frt) arranged in the same direction were inserted on both sides of the resistance gene, forming a Neo cassette. The connection between the 5′ end of Neo cassette and the polyA sequence was designed as

(SEQ ID NO: 9)
5′-aggcggaaagaaccagctggggctcgactagagcttgcggaacc
cttcgaattccgaagttcctattctctagaaagtataggaacttc-3′,

where the last “c” in the sequence “cccttc” is the last nucleotide of the polyA sequence, the “g” in the sequence “gatt” is the first nucleotide of the Neo cassette. The connection between the 3′ end of the Neo cassette and the mouse sequence as

(SEQ ID NO: 10)
5′-gaagttcctattctctagaaagtataggaacttcatcagtcaggta
cataattaggtggatcctagatctccacagccatgctcaaagcccaccc
agcatcagtacaaccc-3′,

where the “c” in the sequence “ggatc” is the last nucleotide of the Neo cassette, and the “c” in the sequence “ctaga” is the first nucleotide of the mouse sequence. In addition, the targeting vector also contains a negative selection marker (the coding sequence for diphtheria toxin A subunit (DTA)) downstream of the 3′ homology arm. The mRNA sequence of the transformed humanized mouse FcRn is shown in SEQ ID NO: 11, and the expressed protein sequence is shown in SEQ ID NO: 2.

The construction of the targeting vector can be carried out by enzyme cleavage and ligation. The constructed targeting vector was preliminarily verified by enzyme digestion, and then sent to a sequencing company for sequencing verification. The targeted vector verified by sequencing was electroporated into embryonic stem cells of C57BL/6 mice, and the obtained cells were screened with the positive clone selection marker (Neo cassette). PCR and Southern Blot technology were used to detect and confirm the integration of exogenous genes. The clones identified as positive by PCR (the primers and the target fragment lengths are shown in Table 2) are then subjected to Southern Blot where the cell DNA was respectively digested with ScaI or BamHI and hybridized with two probes (the probes and the target fragment lengths are shown in Table 1). The results are shown in FIG. 4. The test results showed that 9 clones were identified to be positive by PCR. After further verification by sequencing, 8 out of the 9 clones (with 2-A8 being the exception) were identified as positive clones without random insertion. These 8 positive clones were named 1-G2, 2-A7, 2-D3, 3-B2, 3-D4, 3-G11, 4-B9 and 4-D9.

TABLE 1
Southern Blot probes and target fragment lengths

| Restriction | | Wild-type | Recombinant |
|---|---|---|---|
| endonuclease | Probe | fragment size | fragment size |
| ScaI | 5′ Probe | 9.9 kb | 8.0 kb |
| Bam HI | 3′ Probe | 10.2 kb | 8.7 kb |

The primers for the PCR assay are shown in Table 2.

TABLE 2
PCR primers and target fragment length

| Primer | SEQ ID | Sequence (5′-3′) | Fragment size |
|---|---|---|---|
| F1 | SEQ ID NO: 12 | TGTCAAGTACGAATGTGTATGC | 5653 bp |
| R1 | SEQ ID NO: 13 | ATGACGATTCCCACCACGAG | |

The following Southern Blot probes were used for Southern Blot detection:

5′ Probe:
5′ Probe-F:
(SEQ ID NO: 14)
5′-AGGCAGGCAGATTTCTGACA-3′,
5′ Probe-R:
(SEQ ID NO: 15)
5′-TCCAGAAGCCTGAGTTTAGCT-3′;
3′ Probe:
3′ Probe-F:
(SEQ ID NO: 16)
5′-CGGTTCTTATATTCAGTGAGGAAAC-3′,
3′ Probe-R:
(SEQ ID NO: 17)
5′-CCAATCCTGCACACAGCTAC-3′.

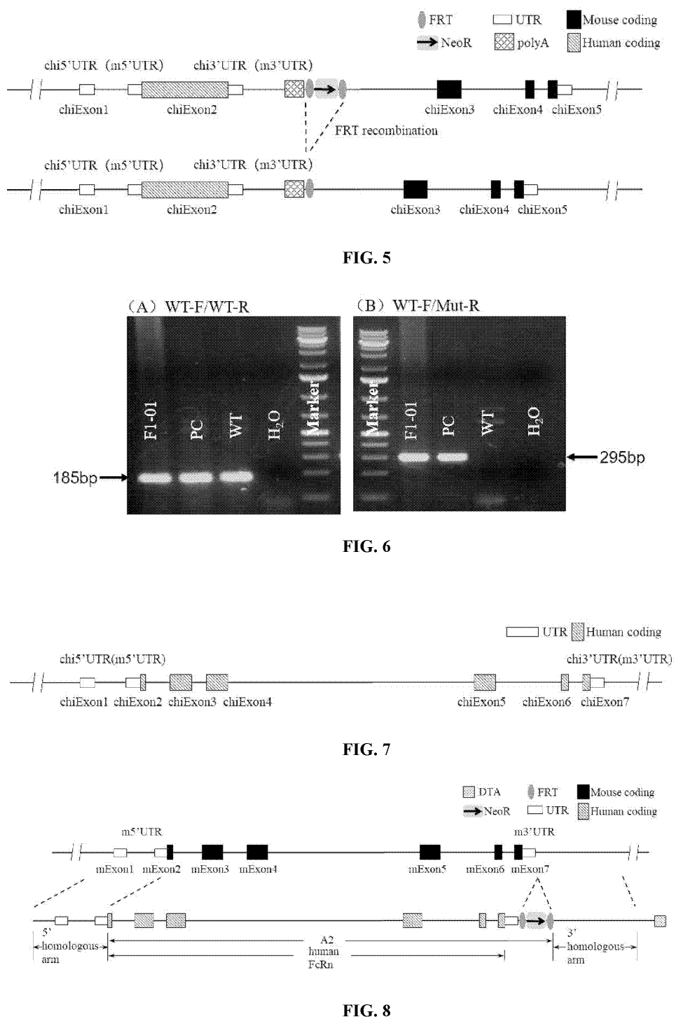

The verified positive clone cells (black mice) were introduced into isolated blastocysts (white mice) according to techniques known in the art, and the obtained chimeric blastocysts were transferred into cell culture media for a short-term culture and then transplanted to the fallopian tubes of female recipient mice (white mice) to produce F0 generation chimeric mice (black and white). The F0 generation chimeric mice were backcrossed with the wild-type mice to obtain the F1 generation mice, and then the F1 generation heterozygous mice were mated to each other to obtain the F2 generation homozygous mice. The positive clone mice can also be mated with the Flp tool mice to remove the positive clone selection marker (see FIG. 5 for the schematic diagram of the process), and then be mated to each other to obtain a humanized FcRn gene homozygous mouse. The somatic cell genotype of the offspring mouse were identified by PCR (primers are shown in Table 3). The PCR results of an exemplary F1 generation mouse (the Neo marker gene has been removed) are shown in FIG. 6. F1-01 mice were positive heterozygous mice. The results indicate that FcRn humanized mice without random insertion can be constructed, and can be stably passed to the offspring.

TABLE 3
Primer names and specific sequences

| Primer | SEQ ID NO. | Sequence (5′-3′) | Fragment size (bp) |
|---|---|---|---|
| WT-F | SEQ ID NO: 18 | CCAGCGTCCTGGTCTACGAA | WT: 185 bp |
| WT-R | SEQ ID NO: 19 | GGTGGCCTCTTACCTGAGCC | |
| WT-F | SEQ ID NO: 18 | CCAGCGTCCTGGTCTACGAA | Mut: 295 bp |
| Mut-R | SEQ ID NO: 20 | GGCTATTGTAGCTCAGGTACTGC | |

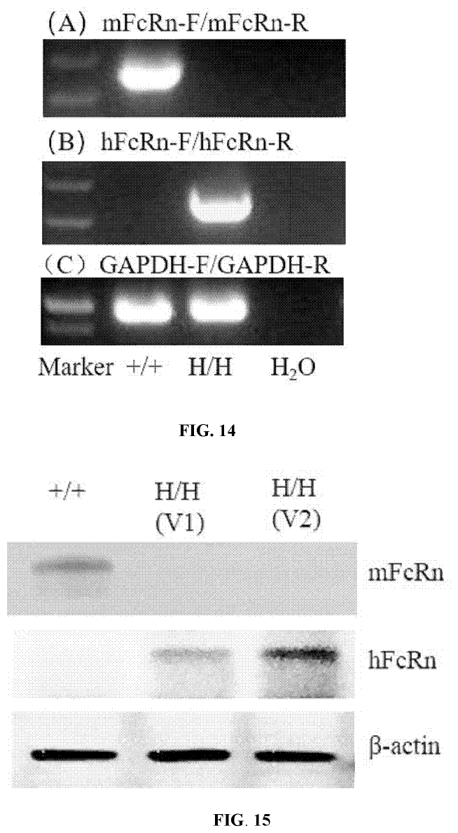

The expression of humanized FcRn mRNA in positive mice can be confirmed by RT-PCR. Specifically, a 6-week-old female C57BL/6 wild-type mouse and one FcRn gene humanized homozygous mouse prepared following the methods in this example were taken. Kidney tissues were collected after euthanasia, and the cell suspension was prepared by grinding. Cellular RNA was extracted according to the TRIzol kit instructions, reverse transcribed into cDNA, and then to perform RT-PCR detection. The detection results are shown in FIG. 14. As can be seen from the figure, murine FcRn mRNA was detected in the kidneys of C57BL/6 wild-type mice (FIG. 14(A)), but humanized FcRn mRNA was not detected (FIG. 14(B)); only humanized FcRn mRNA was detected in kidney tissue of mice homozygous for humanization of the FcRn gene (FIG. 14(B)), and no murine FcRn mRNA was detected (FIG. 14(A)).

RT-PCR primer sequences include:

mFcRn-F:
(SEQ ID NO: 44)
5′-ATCTACGGGGCTTCCCTCTT-3′
mFcRn-R:
(SEQ ID NO: 45)
5′-GCAACAGGTCACCAGAGTCA-3′
hFcRn-F:
(SEQ ID NO: 46)
5′-CTCCTGTACCACCTTACCGC-3′
hFcRn-R:
(SEQ ID NO: 47)
5′-ACACCGATGACGATTCCCAC-3′
GAPDH-F:
(SEQ ID NO: 48)
5′-TCACCATCTTCCAGGAGCGAGA-3′
GAPDH-R:
(SEQ ID NO: 49)
5′-GAAGGCCATGCCAGTGAGCTT-3′

### Example 2. Construction of FcRn Gene Humanized Mice

The 9824 bp nucleotide sequence including a part of exon 2, exons 3-6 and a part of exon 7 of the mouse FcRn gene can be replaced by the corresponding human sequence (12719 bp nucleotide sequence including a part of exon 1, exons 2-5 and a part of exon 6 of the human FcRn gene) by gene editing technology to obtain the humanized FcRn gene sequence (the schematic diagram is shown in FIG. 7).

The specific targeting strategy is shown in FIG. 8. The targeting vector contains the homology arm sequences upstream and downstream of the mouse FcRn gene, and the A2 fragment containing the human FcRn sequence. Among them, the upstream homology arm sequence (5′ homology arm, SEQ ID NO: 21) is the same as the nucleotide sequence from position 45103070 to position 45106859 of NCBI accession number NC_000073.6, and the downstream homology arm sequence (3′ homology arm, SEQ ID NO: 22) is identical to the nucleotide sequence from position 45088115 to position 45092654 of NCBI accession number NC_000073.6. The A2 fragment contains the sequence of a part of exon 2, exons 3-5 and a part of exon 6 of the human FcRn gene (SEQ ID NO: 23), this sequence is the same as the nucleotide sequence from position 49513401 to position 49526119 of the NCBI accession number NC_000019.10. The connection sequence immediately upstream of the human FcRn sequence in the A2 fragment is designed to be

(SEQ ID NO: 24)
5′-agtcacgtgtcccttccactccgggtcaccctgtcggaatggggg
tcccgcggcctcagccctgggcgctggggctcctg-3′,

where the “a” in the sequence “tcgga” is the last nucleotide in the upstream mouse sequence and the “a” in sequence “atggg” is the first nucleoside in the human sequence. The connection sequence immediately downstream of the human FcRn sequence is designed as

(SEQ ID NO: 25)
5′-gctgatttgaaggatgtaaatgtgattccagccaccgcctga
tgcagactcgggccccctgcccactgcagcctttcgggc-3′,

where the “a” in the sequence “gcctga” is the last nucleotide in the human sequence, and the “t” in the sequence “tgcag” is the first nucleotide in the downstream mouse sequence.

The targeting vector also includes a resistance gene for the selection of the positive clones. In this example, the coding sequence of neomycin phosphotransferase (Neo) was used as the resistance genes. Two site-specific recombination systems (Frt) arranged in the same direction were inserted on both sides of the resistance gene, forming a Neo cassette. The connection between the 5′ end of Neo cassette and the polyA sequence is designed as

(SEQ ID NO: 26)
5′-attggattccattacagatggttagccaccatgtggttGTCGAC
GGTATCGATAAGCTTGATATCGAATTCCGAAGTTCCTATTCTCTAGAA
AGTATAGGAACTT-3′,

where the last “t” in the sequence “gtggtt” is the last nucleotide of the mouse sequence, and the first “G” in the sequence “GTCG” is the first nucleotide of the Neo cassette. The connection between the 3′ end of the Neo cassette and the mouse sequence is designed as

(SEQ ID NO: 27)
5′-AAGTTCCTATTCTCTAGAAAGTATAGGAACTTCATCAGTCAGGTA
CATAATGGTGGATCCgctgggacttgaactctggtcctctggaagaaca
gt-3′,

where the last “C” in the sequence “GATCC” is the last nucleotide of the Neo cassette and the first “g” in the sequence “gctgg” is the first nucleotide of the mouse sequence. In addition, the targeting vector also contains a negative selection marker (the coding sequence for diphtheria toxin A subunit (DTA)) downstream of the 3′ homology arm. The mRNA sequence of the transformed humanized mouse FcRn is shown in SEQ ID NO: 11, and the expressed protein sequence is shown in SEQ ID NO: 2.

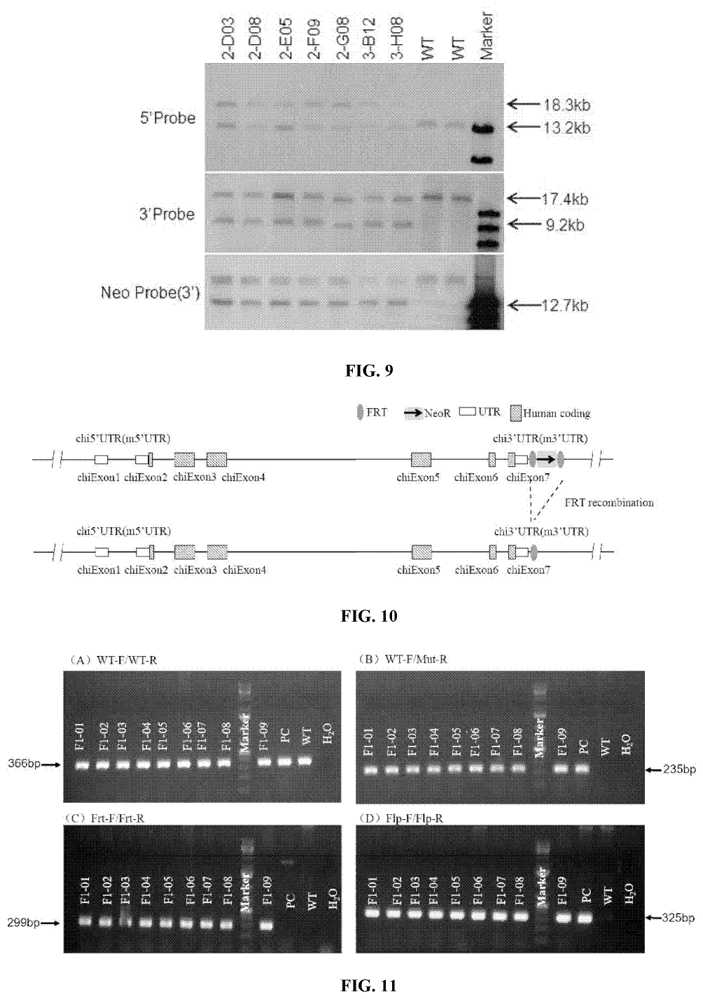

The construction of the targeting vector can be carried out by enzyme cleavage and ligation. The constructed targeting vector was preliminarily verified by enzyme digestion, and then sent to a sequencing company for sequencing verification. The targeted vector verified by sequencing was electroporated into embryonic stem cells of C57BL/6 mice, and the obtained cells were screened with the positive clone selection marker (Neo cassette). PCR and Southern Blot technology were used to detect and confirm the integration of exogenous genes. The clones identified as positive by PCR (the primers and the target fragment lengths are shown in Table 5) were then subjected to Southern Blot where the cell DNA was respectively digested with ScaI or BamHI and hybridized with three probes (the probes and the target fragment lengths are shown in Table 4). The results are shown in FIG. 9. The test results showed that 7 clones were identified to be positive by PCR. After further verification by sequencing, all 7 clones were identified as positive clones without random insertions. These 7 positive clones were named 2-D03, 2-D08, 2-E05, 2-F09, 2-G08, 3-B12 and 3-H08.

TABLE 4
Southern Blot probes and target fragment lengths

| Restriction | | Wild-type | Recombinant |
|---|---|---|---|
| endonuclease | Probe | fragment size | fragment size |
| XbaI | 5′ Probe | 13.2 kb | 18.3 kb |
| StuI | 3′ Probe | 17.4 kb | 9.2 kb |
| XmnI | Neo Probe(3′) | — | 12.7 kb |

TABLE 5
PCR primers and target fragment length

| Primer | SEQ ID | Sequence (5′-3′) | Fragment size |
|---|---|---|---|
| F1 | SEQ ID NO: 28 | CAGATAGAGCAAGCCGTGGAGAG | 4044 bp |
| Mut-R | SEQ ID NO: 29 | CTCCCAGGAAGGAGAAAGAGCAGG | |
| F2 | SEQ ID NO: 30 | AACTGTTCGCCAGGCTCAAG | 5482 bp |
| R2 | SEQ ID NO: 31 | CACCTCTCAAGTGCTGAGACTGAG | |

The following Southern Blot probes were used for Southern Blot detection:

5′Probe:
5′Probe-F:
(SEQ ID NO: 32)
5′-AACCTGGAGCTCACTGGTCAGCAAA-3′;
5′Probe-R:
(SEQ ID NO: 33)
5′-CACGGGCAGACTCTCAGTTCCATC-3′;
3′Probe:
3′Probe-F:
(SEQ ID NO: 34)
5′-ATAGCCCAGAGCTCAAGGATAGGCA-3′;
3′Probe-R:
(SEQ ID NO: 35)
5′-CCCAGTCTGCTTTCTGTCCTGTCAC-3′;
Neo Probe (3′)
Neo Probe (3′)-F:
(SEQ ID NO: 36)
5′-GGATCGGCCATTGAACAAGAT-3′,
Neo Probe (3′)-R:
(SEQ ID NO: 37)
5′-CAGAAGAACTCGTCAAGAAGGC-3′.

The verified positive clone cells (black mice) were introduced into isolated blastocysts (white mice), and the obtained chimeric blastocysts were transferred into cell culture media for a short-term culture and then transplanted to the fallopian tubes of female recipient mice (white mice) to produce F0 generation chimeric mice (black and white). The F0 generation chimeric mice were backcrossed with the wild-type mice to obtain the F1 generation mice, and then the F1 generation heterozygous mice were mated to each other to obtain the F2 generation homozygous mice. The positive clone mice were mated with the Flp tool mice to remove the positive clone selection marker (see FIG. 10 for the schematic diagram of the process), and then be mated to each other to obtain a humanized FcRn gene homozygous mouse. The somatic cell genotype of the offspring mouse were identified by PCR (primers are shown in Table 6). The PCR results of an exemplary F1 generation mouse (the Neo marker gene has been removed) are shown in FIG. 11. Nine mice (numbered as F1-01, F1-02, F1-03, F1-04, F1-05, F1-06, F1-07, F1-08 and F1-09) were positive heterozygous mice. The results indicate that FcRn humanized mice without random insertion can be constructed, and can be stably passed to offspring.

TABLE 6
Primer names and specific sequences

| Primer | SEQ ID NO. | Sequence (5′-3′) | Fragment size (bp) |
|---|---|---|---|
| WT-F | SEQ ID NO: 38 | TGAGAAGGTGAAAGTTCACAGAGGA | WT: 366 bp |
| WT-R | SEQ ID NO: 39 | CCCACAGTGAGATTTCCATGAAGGC | |
| WT-F | SEQ ID NO: 38 | TGAGAAGGTGAAAGTTCACAGAGGA | Mut: 235 bp |
| Mut-R | SEQ ID NO: 29 | CTCCCAGGAAGGAGAAAGAGCAGG | |
| Frt-F | SEQ ID NO: 40 | TCGATAAGCTTGATATCGAATTCCGAAG | Mut: 299 bp |
| Frt-R | SEQ ID NO: 41 | TCCGTTTCAGAAAAGAGGAGGTCGG | (after Neo removal) |
| Flp-F | SEQ ID NO: 42 | GACAAGCGTTAGTAGGCACATATAC | Mut: 325 bp |
| Flp-R | SEQ ID NO: 43 | GCTCCAATTTCCCACAACATTAGT | |

### Example 3 Western Blot Protein Detection

The expression of the human or humanized FcRn protein in FcRn gene humanized mice was determined by Western Blot. 8-week-old female wild-type C57BL/6 mice (+/+), FcRn gene humanized homozygous mice prepared according to Example 1 (H/H(V1)), and the FcRn gene humanized homozygous mice prepared according to Example 2 (H/H(V2)) were used for this experiment. The mice were euthanized, and samples from the liver, heart, spleen, lung, kidney, and intestine were collected. Anti-human FcRn antibody (hFcRn) and anti-mouse FcRn antibody (mFcRn) were used for Western Blot detection. The detection results are shown in FIGS. 15 & 16.

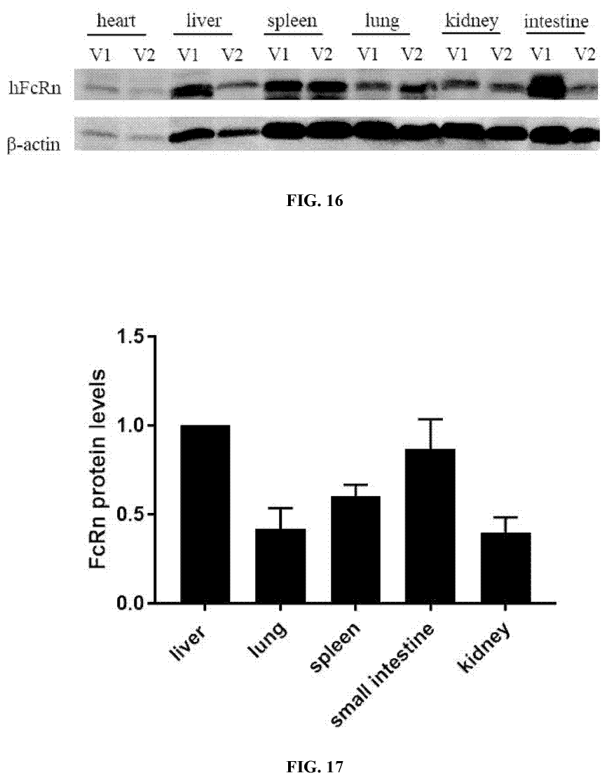

As can be seen from FIG. 15, the expression of murine FcRn was only detected in the liver tissue of wild-type C57BL/6 mice, and human FcRn was not detected. The expression of human FcRn was detected in the liver tissue of FcRn gene humanized homozygous mice (V1 and V2), and the expression of murine FcRn was not detected. FIG. 16 shows that human FcRn protein was detected in all of the heart, liver, spleen, lung, kidney and intestine tissues of FcRn gene humanized homozygous mice (V1 and V2). The results show that human FcRn protein was expressed in FcRn gene humanized mice. FIG. 17 shows the relative level of expression of the human FcRn protein in the liver, spleen, lung, kidney and intestinal tissues of FcRn gene humanized mice prepared according to Example 1.

### Example 4. Pharmacokinetics of Different Antibodies in Mice

Double-antibody sandwich ELISA was used to measure the pharmacokinetics (PK) behavior of different anti-human PD-1 antibodies in FcRn humanized homozygous mice (H/H) prepared according to Example 1 and C57BL/6 wild-type mice (+/+). The PD-1 antibodies tested included Keytruda® (Pembrolizumab) and AB1 (Pembrolizumab biosimilar). Specifically, eight 8-week-old female FcRn humanized mice and four C57BL/6 wild-type mice were used. The mice were fasted and fed with water for 12 h, and then treated with different anti-human PD-1 antibodies Keytruda® or AB1 by tail vein injection. 60 μL of blood was collected from the orbital venous sinus of each mouse at the following time points: 4 days before administration, 0.25 days, 3 days, 5 days, 7 days, 9 days, 12 days, 16 days, 19 days, 22 days, 26 days, 30 days after administration. Serum was collected by centrifugation and was frozen for later use. The specific grouping and dosing schedules are shown in Table 7.

TABLE 7
Grouping and dosing schedule

| | | | | Route of | Administration |
|---|---|---|---|---|---|
| Group | Animal/Number | Drug | Dosage | Administration | Frequency |
| G1 | FcRn humanized mice/4 | Keytruda ® | 10 mg/kg | IV | single dose |
| G2 | C57BL/6 wild-type mice/4 | Keytruda ® | 10 mg/kg | IV | single dose |
| G3 | FcRn humanized mice/4 | AB1 | 10 mg/kg | IV | single dose |

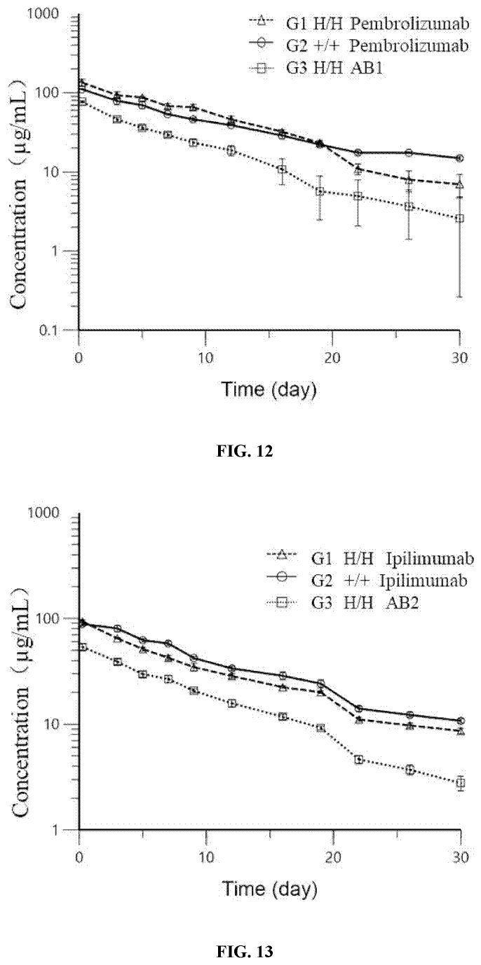

Double-antibody sandwich ELISA was used to determine the concentration of anti-human PD-1 antibody Keytruda® or AB1 in the serum samples. A 96-well ELISA plate was coated with the affinity-purified goat anti-human IgG capture antibody (AffiniPure Goat Anti-Human IgG (H+L)), and excess capture antibodies were removed by PBS washing. Serum samples were added, followed by horseradish peroxidase (HRP) labeled detection antibody (Peroxidase AffiniPure F(ab′)2 Fragment Goat Anti-Human IgG Fc) and the chromogenic HRP substrate. The blood concentration-time curve was drawn according to the ELISA data. FIG. 12 shows the anti-human PD-1 antibody concentration in mouse serum at each time point in the three groups. It can be seen from the figure that the pharmacokinetics profile of Keytruda® in FcRn humanized mice was basically the same as that of C57BL/6 mice (G1 and G2). Different PD-1 antibodies (Keytruda® and AB1) have the same pharmacokinetics trend in FcRn humanized mice (G1 and G3). The data indicate that the anti-human PD-1 antibodies have normal pharmacokinetic parameters in the FcRn humanized mice.

Phoenix Winnolin 8.0 was used to establish a Non-Compartmental Analysis (NCA) model to calculate PK parameters: half-life (T_{1/2}), peak drug concentration (Cₘₐₓ), area under curve for the plasma concentration-time curve at 0-30 days (AUC₀₋₃₀), the area under curve for the plasma concentration-time curve from administration to theoretically extrapolated infinity (AUC_{0-obs}), the apparent volume of distribution (Vd), and the clearance rate (Cl). The overall pharmacokinetic parameters of anti-human PD-1 antibodies in mice of different groups (G1, G2, and G3) are shown in Table 8.

TABLE 8
Overall pharmacokinetic parameters of anti-human PD-1 antibodies

| | | | | | | Cl |
|---|---|---|---|---|---|---|
| | T_{1/2} | Cₘₐₓ | AUC₀₋₃₀ | AUC_{0-obs} | Vd | (mL/ |
| Group | (day) | (ng/mL) | (day*ng/mL) | (day*ng/mL) | (mL/kg) | day/kg) |
| G1 | 7.18 | 134.60 | 1331.21 | 1449.24 | 71.96 | 6.92 |
| G2 | 10.83 | 110.64 | 1196.51 | 1430.63 | 109.63 | 7.02 |
| G3 | 7.43 | 77.08 | 555.17 | 686.28 | 153.65 | 15.27 |

As can be seen from the table, the half-lives of Keytruda® in FcRn humanized mice and C57BL/6 mice were 7.18 days and 10.83 days, respectively. The half-life of AB1 in FcRn humanized mice was 7.43 days. Overall, the half-life of anti-human PD-1 antibodies in C57BL/6 mice is higher than that in FcRn humanized mice. There is only a slight difference between the half-life values of different anti-human PD-1 antibodies in FcRn gene humanized mice. In terms of clearance rate, the clearance rate of Keytruda® is 6.92 mL/day/kg, which is significantly less than the clearance rate of AB1 (15.27 mL/day/kg). The differences in PK parameters between G1 and G3 are attributable to different drugs (Keytruda® and AB1) considering that the route of administration, dose, and animal strain did not change, indicating that FcRn humanized mice can be used to evaluate the pharmacokinetic parameters of different anti-human antibodies.

The same method was used to detect the PK behaviors of anti-human CTLA4 antibody Yevoy® (Ipilimumab) and AB2 (Ipilimumab biosimilar) in FcRn humanized homozygous mice (H/H) and C57BL/6 wild-type mice (+/+). The specific grouping and dosing schedules are shown in Table 9.

TABLE 9
Grouping and dosing schedule

| | | | | Route of | Administration |
|---|---|---|---|---|---|
| Group | Animal/Number | Drug | Dosage | Administration | Frequency |
| G1 | FcRn humanized mice/4 | Yevoy ® | 10 mg/kg | IV | Single Dose |
| G2 | C57BL/6 wild-type mice/4 | Yevoy ® | 10 mg/kg | IV | Single Dose |
| G3 | FcRn humanized mice/4 | AB2 | 10 mg/kg | IV | Single Dose |

The double-antibody sandwich ELISA method was again used to detect the concentrations of anti-human CTLA4 antibody Yevoy® or AB2 in each serum sample, and the blood concentration-time curve was drawn according to the ELISA data (as shown in FIG. 13). It can be seen from the figure that the PK trends of the anti-human CTLA4 antibodies Yevoy® and AB2 in the FcRn humanized mice were basically the same as those in the C57BL/6 mice, indicating that the anti-human CTLA4 antibodies have normal pharmacokinetic parameters in the FcRn humanized mice.

Phoenix Winnolin 8.0 was used to establish an NCA model to calculate the overall pharmacokinetic parameters, as shown in Table 10.

TABLE 10
Overall pharmacokinetic parameters
of anti-human CTLA4 antibodies

| | | | | | | Cl |
|---|---|---|---|---|---|---|
| | T_{1/2} | Cₘₐₓ | AUC₀₋₃₀ | AUC_{0-obs} | Vd | (mL/ |
| Group | (day) | (ng/mL) | (day*ng/mL) | (day*ng/mL) | (mL/kg) | day/kg) |
| G1 | 9.12 | 93.70 | 914.24 | 1028.22 | 128.01 | 9.75 |
| G2 | 9.13 | 90.74 | 1091.54 | 1234.11 | 106.66 | 8.11 |
| G3 | 6.96 | 53.79 | 505.479 | 534.08 | 188.39 | 18.81 |

From the table, the half-life values of anti-human CTLA4 antibody Yevoy® in FcRn humanized mice and C57BL/6 mice were 9.12 days and 9.13 days, respectively, and the half-life of AB2 in FcRn humanized mice was 6.96 days. Overall, the half-life values of anti-human CTLA4 antibodies in C57BL/6 mice were slightly higher than those in FcRn humanized mice, and the clearance rate in C57BL/6 mice was relatively low at 8.11 mL/day/kg. The half-life and clearance rate of different anti-human CTLA4 antibodies in FcRn humanized mice also showed some differences. The half-life of Yevoy® in FcRn humanized mice was greater than that of AB2, and the clearance rate of Yevoy® (9.75 mL/day/kg) was significantly lower than that of AB2 (18.81mL/day/kg). Considering the same route of administration, dosage and animal strain, this difference in pharmacokinetic parameters between G1 and G3 was attributable to different drugs. The data confirms that FcRn humanized mice can be used to evaluate the pharmacokinetic parameters of different human or humanized antibodies.

### Example 5 Pharmacokinetics of Different Antibody Subclasses in Humanized Mice

The same method discussed in Example 4 was used to evaluate the pharmacokinetic parameters of different subclasses (AB3-IgG1, AB3-IgG2, AB3-IgG4) of antibody AB3 (obtained by immunizing mice using conventional methods, see Janeway's Immunobiology (9th Edition)) in homozygous FcRn humanized mice (H/H) and C57BL/6 mice (+/+). The grouping and dosing schedules are shown in Table 11.

TABLE 11
Grouping and dosing schedule

| | | | | Route of | Administration |
|---|---|---|---|---|---|
| Group | Animal/Number | Drug | Dosage | Administration | Frequency |
| G1 | FcRn humanized mice/4 | AB3-IgG1 | 10 mg/kg | IV | Single Dose |
| D1 | C57BL/6 mice/4 | | | | |
| G2 | FcRn humanized mice/4 | AB3-IgG2 | 10 mg/kg | IV | Single Dose |
| D2 | C57BL/6 mice/4 | | | | |
| G3 | FcRn humanized mice/4 | AB3-IgG4 | 10 mg/kg | IV | Single Dose |
| D3 | C57BL/6 mice/4 | | | | |

The overall pharmacokinetic parameters of the antibodies in each group of mice are shown in Table 12.

TABLE 12
Overall pharmacokinetic parameters of IgG1,
IgG2 and IgG4 subclasses of antibody AB3

| | T_{1/2} | Cₘₐₓ | AUC₀₋₅₇₆ | AUC_{0-obs} | Vd | Cl |
|---|---|---|---|---|---|---|
| Group | (h) | (ug/mL) | (h*ug/mL) | (h*ug/mL) | (mL/kg) | (mL/h/kg) |
| G1 | 82.22 | 56.92 | 5475.00 | 5636.36 | 207.03 | 1.86 |
| D1 | 186.09 | 41.68 | 7004.5 | 8151.68 | 365.88 | 0.85 |
| G2 | 71.20 | 136.49 | 12225.22 | 12405.75 | 81.76 | 0.81 |
| D2 | 150.16 | 177.78 | 25568.94 | 28610.37 | 69.89 | 0.36 |
| G3 | 66.79 | 52.56 | 10347.34 | 11146.95 | 80.90 | 0.94 |
| D3 | 150.16 | 49.18 | 12443.77 | 14009.95 | 151.48 | 0.73 |

As indicated in the table, the half-life values of IgG1, IgG2 and IgG4 subclasses of antibody AB3 in C57BL/6 mice were higher than those in FcRn humanized mice. There was only slight difference among the half-life values of IgG1, IgG2 and IgG4 subclasses of antibody AB3 in in FcRn humanized mice. The clearance rate in FcRn humanized mice was higher than that in C57BL/6 mice. Considering the same administration method, dosage, and animal strain, this difference in pharmacokinetic parameters between G1 and G3 was attributable to different drugs, indicating that FcRn humanized mice can be used to evaluate the pharmacokinetics of different subclasses of antibodies.

### Example 6. Pharmacokinetic Detection of YTE Mutant Antibodies Ab1 and Ab2 in Mice

The same method discussed in Example 4 was used to evaluate the pharmacokinetic parameters of anti-human antibodies Ab1, Ab1-YTE, Ab2, Ab2-YTE (obtained by immunizing mice using conventional methods, see Janeway's Immunobiology (9th Edition)) in FcRn humanized mice. YTE mutation refers to the combination of a methionine 252 to tyrosine (M252Y) mutation, a serine 254 to threonine (S254T) mutation and a threonine 256 to glutamic acid (T256E) mutation in the Fc constant region of the antibody (EU numbering).

Each group contained 5 mice. Each mouse was administered by tail vein injection at a single dose of 100 ug/mL.

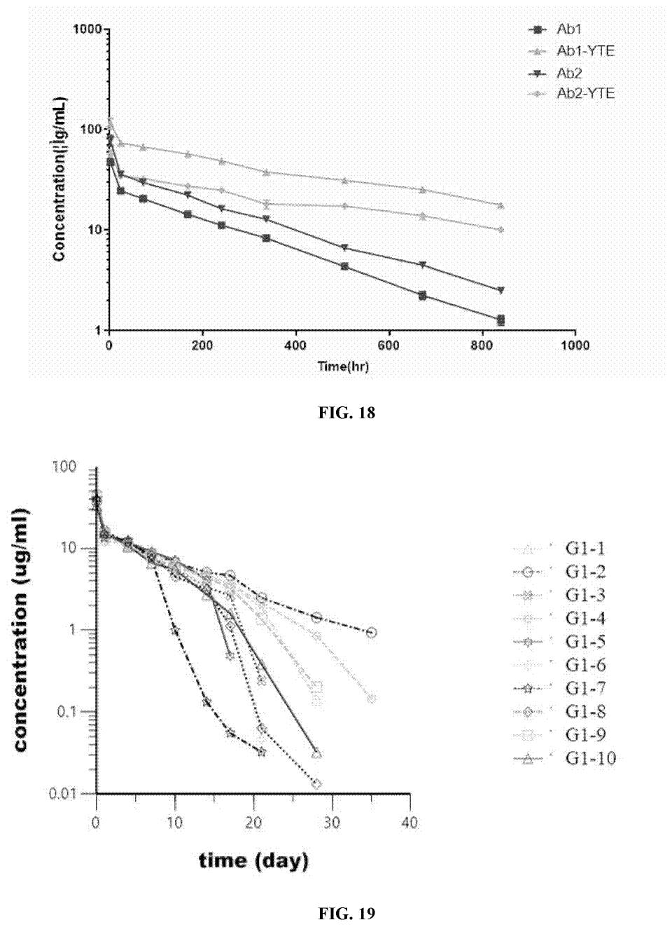

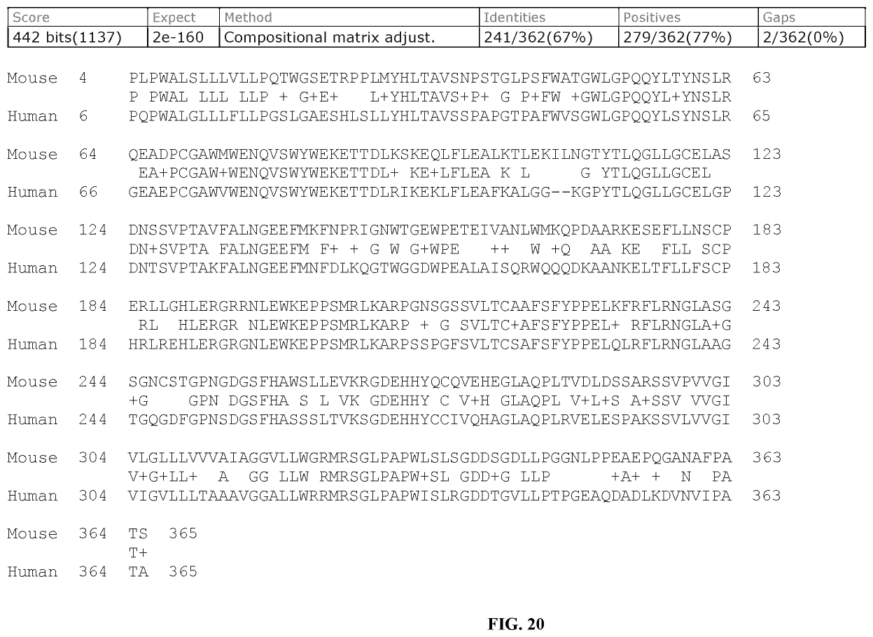

The mouse serum antibody concentrations at each time point in the 4 groups are shown in FIG. 18. It can be seen from the figure that the PK trends of antibodies Ab1-YTE and Ab2-YTE in FcRn humanized mice were basically the same as that of Ab1 and Ab2, but the metabolic rates of antibodies Ab1-YTE and Ab2-YTE were lower than those of Ab1 and Ab2. The data indicates that the YTE mutation in Ab1-YTE and Ab2-YTE antibodies can prolong the circulation time of antibodies. The overall pharmacokinetic parameters of the four groups of antibodies Ab1, Ab1-YTE, Ab2 and Ab2-YTE in mice are shown in Table 13.

TABLE 13
Overall pharmacokinetic parameters of antibodies
Ab1, Ab1-YTE, Ab2 and Ab2-YTE

| | | | | | | Cl |
|---|---|---|---|---|---|---|
| | T_{1/2} | Cₘₐₓ | AUC₀₋₃₃₆ | AUC_{0-obs} | Vd | (mL/ |
| Group | (h) | (ug/mL) | (h*ug/mL) | (h*ug/mL) | (mL/kg) | h/kg) |
| Ab1 | 213.33 | 47.26 | 5429.70 | 7984.44 | 3.862 | 0.013 |
| Ab1-YTE | 303.84 | 115.51 | 19498.94 | 35986.4 | 1.221 | 0.003 |
| Ab2 | 211.19 | 77.91 | 8177.58 | 12067.45 | 2.533 | 0.008 |
| Ab2-YTE | 369.98 | 56.96 | 9501.65 | 20180.20 | 2.507 | 0.006 |

As shown in the table, overall, the half-lives of mutant antibodies (Ab1-YTE and Ab2-YTE) in FcRn humanized mice were longer than those of wildtype antibodies (Ab1 and Ab2). The clearance rates of the mutant antibodies (Ab1-YTE and Ab2-YTE) were lower than those of wildtype antibodies (Ab1 and Ab2). The results showed that the FcRn humanized mice can be used to evaluate the pharmacokinetics of antibodies and mutant antibodies.

### Example 7 Pharmacokinetic Detection of AB4 in Different Mice

Anti-human antibody AB4 (obtained by immunizing mice using conventional methods, see Janeway's Immunobiology (9th Edition) was tested. Pharmacokinetic parameters were evaluated in FcRn humanized mice using the same method discussed in Example 4. 8-10 weeks old FcRn humanized homozygous female mice prepared according to Example 1 (H/H(V1)) and FcRn humanized homozygous female mice prepared according to Example 2 (H/H(V2)) were used. The mice were grouped and injected with AB4 according to Table 14. 80-100 μL of retro-orbital blood was collected from each mouse at the following time points: 15 minutes, 24 hours, 4 days, 7 days, 10 days, 14 days, 17 days, 21 days, 28 days, and 35 days after administration. Blood serum was collected by centrifugation and was frozen for testing.

TABLE 14
Grouping and dosing schedule

| | Animal/ | | | Route of | Administration |
|---|---|---|---|---|---|
| Group | Number | Drug | Dosage | Administration | Frequency |
| G1 | H/H(V1)/10 | AB4 | 3 mg/kg | IV | Single Dose |
| G2 | H/H(V2)/10 | AB4 | 3 mg/kg | IV | Single Dose |

The overall pharmacokinetic parameters of antibody AB4 in mice in groups G1 and G2 are shown in Table 15. An exemplary single mouse drug concentration-time curve (G1 group) is shown in FIG. 19.

TABLE 15
Overall pharmacokinetic parameters of antibody Ab4

| | | | | | | Cl |
|---|---|---|---|---|---|---|
| | T_{1/2} | Cₘₐₓ | AUC₀₋ₜ | AUC_{0-obs} | Vd | (mL/ |
| Group | (day) | (ug/mL) | (day*ug/mL) | (day*ug/mL) | (mL/kg) | day/kg) |
| G1 | 3.34 | 40.3 | 153.61 | 155.56 | 87.5 | 19.79 |
| G2 | 3.20 | 44.03 | 95.28 | 95.88 | 139.31 | 32.90 |

As shown in Table 15 and FIG. 19, the half-life (T_{1/2}) values and the highest blood drug concentrations (Cₘₐₓ) in the G1 and the G2 were not much different. The mice in the G2 had a larger apparent volume of distribution (Vd) and a relatively fast clearance rate (Cl) comparing to G1. The AUC in the G2 group was lower than that in the G1 group. The results indicate that the FcRn humanized mice prepared according to Example 1 and Example 2 can be used to evaluate the pharmacokinetic parameters of antibody drugs.

### Example 8. Preparation of Double Humanized or Multiple Humanized Mice

Using the disclosed method, a mouse model with double humanized genes or multiple humanized genes can also be prepared. For example, in Example 1 or 2, the embryonic stem cells used for blastocyst microinjection can be have genetic modifications on other genes such as H2-D, B2M, PD-1, PD-L1, CTLA4, B7H3, B7H4, CD47 and IL23A. Alternatively, in FcRn humanized mice, other genes can be modified using mouse ES embryonic stem cells and gene recombination technology to obtain mice with double humanized genes or multiple humanized genes. The homozygous or heterozygous FcRn humanized mice obtained by this method can also be mated with other gene-modified homozygous or heterozygous mice. According to the Mendel's Laws of Heredity, there is a probability to obtain mice with double humanized genes or multiple humanized genes. The heterozygous mice can be mated with each other to obtain homozygous mice with double humanized genes or multiple humanized genes. These double-gene or multi-gene modified mice can be used for in vivo efficacy validation of drugs targeting human FcRn and other drug targets.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A genetically-modified, non-human animal whose genome comprises at least one chromosome comprising a sequence encoding a human or chimeric neonatal Fc receptor for IgG (FcRn).

2. The animal of claim 1, wherein the sequence encoding the human or chimeric FcRn is operably linked to an endogenous regulatory element at the endogenous FcRn gene locus in the at least one chromosome.

3. The animal of claim 1 or 2, wherein the sequence encoding the human or chimeric FcRn comprises a sequence encoding an amino acid sequence that is at least 70%, 75%, 80%, 85%, 90%, 95%, 99%, or 100% identical to SEQ ID NO: 2.

4. The animal of any one of claims 1-3, wherein the sequence encoding the human or chimeric FcRn is operably linked to an endogenous 5′-UTR (e.g., immediately after 5′-UTR).

5. The animal of any one of claims 1-4, wherein the animal is a mammal, e.g., a monkey, a rodent or a mouse.

6. The animal of any one of claims 1-5, wherein the animal is a mouse or a rat.

7. The animal of any one of claims 1-6, wherein the animal does not express endogenous FcRn or expresses a decreased level of endogenous FcRn as compared to that of an animal without genetic modification.

8. The animal of any one of claims 1-7, wherein the animal has one or more cells expressing human or chimeric FcRn.

9. The animal of any one of claims 1-8, wherein the sequence encoding the human or chimeric FcRn comprises a part of exon 1, all of exon2, all of exon 3, all of exon 4, all of exon 5 and a part of exon 6 of the human FcRn nucleotide sequence, wherein the part of exon 1 contains at least 50 bp of nucleotides, and the part of exon 6 contains at least 80 bp of the human FcRn nucleotide sequence.

10. The animal of any one of claims 1-9, wherein genetically-modified non-human animal comprises all of exon 1, a part of exon 2, all of exon 5, all of exon 6 and all of exon 7 of the non-human animal's endogenous FcRn gene.

11. The animal of any one of claims 1-10, wherein the human or chimeric FcRn protein comprises an amino acid sequence that are at least 70%, 75%, 80%, 85%, 90%, 91%, 92%, 93%, 94%, 95%, 96%, 97%, 98%, 99% or 100% identical to the amino acid sequences encoded by SEQ ID NO: 5, SEQ ID NO: 11 or SEQ ID NO: 23.

12. The animal of any one of claims 1-11, wherein the sequence encoding the human or chimeric FcRn contains a nucleotide sequence that are at least 70%, 75%, 80%, 85%, 90%, 91%, 92%, 93%, 94%, 95%, 96% %, 97%, 98% or at least 99% identical to the nucleotide sequence shown in SEQ ID NO: 6, SEQ ID NO: 7, SEQ ID NO: 8, SEQ ID NO: 9, SEQ ID NO: 24, SEQ ID NO: 25, SEQ ID NO: 26, or SEQ ID NO: 27.

13. The non-human animal according to claim 1-12, wherein the human or chimeric FcRn gene further comprises all of exon 1, a part of exon 2 and a part of exon 7 of the non-human animal's endogenous FcRn gene.

14. A genetically-modified, non-human animal, wherein the genome of the animal comprises an insertion of a sequence encoding a region of human FcRn or chimeric FcRn at an endogenous FcRn gene locus.

15. The animal of claim 14, wherein the inserted sequence is operably linked to an endogenous regulatory element at the endogenous FcRn locus, and one or more cells of the animal express human FcRn or chimeric FcRn.

16. The animal of claim 14 or 15, wherein the animal does not express endogenous FcRn or expresses a decreased level of endogenous FcRn as compared to that of an animal without genetic modification.

17. The animal of any one of claims 14-16, wherein the inserted sequence is located immediately after 5′-UTR at the endogenous FcRn locus.

18. The animal of any one of claims 14-17, wherein the animal has one or more cells expressing a chimeric FcRn having a humanized extracellular region, transmembrane region, and/or cytoplasmic region, wherein the humanized extracellular region comprises a sequence that is at least 50%, 60%, 70%, 80%, 90%, 95%, or 99% identical to the corresponding extracellular region of human FcRn.

19. The animal of any one of claims 14-18, wherein the human or chimeric FcRn comprises a sequence that is at least 70%, 75%, 80%, 85%, 90%, 91%, 92%, 93%, 94%, 95%, 96%, 97%, 98% or at least 99% identical to SEQ ID NO: 2.

20. The animal of any one of claims 14-19, wherein the genome of the animal comprises at least SEQ ID NO: 6, SEQ ID NO: 7, SEQ ID NO: 24, SEQ ID NO: 25, or a nucleotide sequence that is at least 70%, 75%, 80%, 85%, 90%, 91%, 92%, 93%, 94%, 95%, 96%, 97%, 98% or at least 99% identical to the nucleotide sequence set forth in SEQ ID NO: 6, SEQ ID NO: 7, SEQ ID NO: 24 or SEQ ID NO: 25.

21. The animal of any one of claims 14-20, wherein the animal further comprising a deletion of one or more nucleotide from the endogenous FcRn gene.

22. The animal of any one of claims 14-21, wherein the animal FcRn further comprises an endogenous FcRn 3′-UTR and a polyA sequence.

23. The animal of any one of claims 14-22, wherein the animal is heterozygous or homozygous with respect to the insertion at the endogenous FcRn gene locus.

24. A method for making a genetically-modified, non-human animal, comprising:
inserting in at least one cell of the animal, at an endogenous FcRn gene locus, a sequence encoding at least a region of human FcRn gene.

25. The method of claim 24, wherein the sequence encoding the region of human FcRn gene comprises exon 1, exon 2, exon 3, exon 4, exon 5, exon 6, or a part thereof, of a human FcRn gene.

26. The method of claim 24 or 25, wherein the sequence encoding a region of human FcRn gene encodes a sequence that is at least 90% identical to SEQ ID NO: 2.

27. The method of any one of claims 24-26, wherein the sequence encoding a region of human FcRn gene is 90% identical to SEQ ID NO: 5 or SEQ ID NO: 23.

28. The method of any one of claims 24-27, wherein the sequence encoding a region of human FcRn gene is 100% identical to SEQ ID NO: 5 or SEQ ID NO: 23

29. The method of any one of claims 24-28, wherein the method further comprises deleting one or more nucleotides of the endogenous FcRn gene.

30. The method of any one of claims 24-29, wherein a part of exon 1, all of exon 2, all of exon 3, all of exon 4, all of exon 5 and a part of exon 6 of the human FcRn gene are inserted or substituted into the non-human animal's endogenous FcRn gene locus, wherein the part of exon 1 of the human FcRn gene comprises at least 50 bp contiguous human nucleotides, wherein the part of exon 6 of the human FcRn gene comprises at least 80 bp contiguous human nucleotides.

31. The method of any one of claims 24-30, wherein a part of exon 2, exons 3-6 and a part of exon 7 of the endogenous mouse FcRn gene are replaced with a nucleotide sequence encoding the human or chimeric FcRn.

32. The method of any one of claims 24-31, wherein a part of exon 2, all of exon 3, all of exon 4 of the endogenous mouse FcRn gene are replaced with a nucleotide sequence encoding the human or chimeric FcRn.

33. A non-human animal comprising at least one cell comprising a nucleotide sequence encoding a humanized FcRn polypeptide, wherein the humanized FcRn polypeptide comprises at least 50 contiguous amino acid residues that are identical to the corresponding contiguous amino acid sequence of a human FcRn, wherein the animal expresses the humanized FcRn.

34. The animal of claim 33, wherein the humanized FcRn polypeptide has at least 10 contiguous amino acid residues that are identical to the corresponding contiguous amino acid sequence of a human FcRn extracellular region.

35. The animal of claim 33 or 34, wherein the humanized FcRn polypeptide comprises a sequence that is at least 90%, 95%, or 99% identical to SEQ ID NO: 2.

36. The animal of any one of claims 33-35, wherein the nucleotide sequence is operably linked to an endogenous FcRn regulatory element of the animal (e.g., 5′-UTR).

37. The animal of any one of claims 33-36, wherein the humanized FcRn polypeptide comprises a humanized extracellular region, a humanized FcRn transmembrane region and/or a humanized FcRn cytoplasmic region.

38. The animal of any one of claims 33-37, wherein the nucleotide sequence is integrated to an endogenous FcRn gene locus of the animal.

39. A method of making a genetically-modified mouse cell that expresses a human FcRn or a chimeric FcRn, the method comprising:
inserting at an endogenous mouse FcRn gene locus, a nucleotide sequence encoding a human FcRn or a chimeric FcRn, thereby generating a genetically-modified mouse cell that includes a nucleotide sequence that encodes the human FcRn or the chimeric FcRn, wherein the mouse cell expresses the human FcRn or the chimeric FcRn.

40. The method of claim 39, wherein the entire coding sequence of human FcRn gene is inserted at the endogenous mouse FcRn gene locus.

41. The method of claim 39 wherein the chimeric FcRn comprises:
the extracellular region of human FcRn; and
the transmembrane region; and/or
the cytoplasmic region of mouse FcRn.

42. The animal of any one of claims 1-23 and 33-38, wherein the animal further comprises a sequence encoding an additional human or chimeric protein.

43. The animal of claim 42, wherein the additional human or chimeric protein is H2-D, B2M, PD-1, PD-L1, CTLA4, B7H3, B7H4, CD47 or IL23A.

44. The method of any one of claims 24-32 and 39-41, wherein the animal or mouse further comprises a sequence encoding an additional human or chimeric protein.

45. The method of claim 44, wherein the additional human or chimeric protein is H2-D, B2M, PD-1, PD-L1, CTLA4, B7H3, B7H4, CD47 or 1L23A.

46. A method of determining effectiveness of a therapeutic agent targeting FcRn for the treatment of an immune-related disease, comprising:
administering the therapeutic agent targeting FcRn to the animal of any one of claims 1-23 and 33-38; and
determining the effects of the therapeutic agent targeting FcRn to the immune-related disease of the animal.

47. The method of claim 46, wherein the immune-related disease is an autoimmune disease.

48. The method of claim 46, wherein determining the effects of the therapeutic agent targeting FcRn to the immune-related disease of the animal comprises measuring the pharmacokinetic parameters of an antibody.

49. The method of claim 48, wherein the pharmacokinetic parameters include half-life (T_{1/2}), peak drug concentration (Cₘₐₓ), area under curve for the plasma concentration-time curve at 0-30 days (AUC₀₋₃₀), the area under curve for the plasma concentration-time curve from administration to theoretically extrapolated infinity (AUC_{0-obs}), the apparent volume of distribution (Vd), and the clearance rate (Cl).

50. A method for evaluating pharmacokinetics of an antibody, comprising
administering the antibody to the animal of any one of any one of claims 1-23 and 33-38; and
determining one or more pharmacokinetic parameters of the antibody.

51. The method of claim 50, wherein the pharmacokinetic parameters include half-life (T_{1/2}), peak drug concentration (Cₘₐₓ), area under curve for the plasma concentration-time curve at 0-30 days (AUC₀₋₃₀), the area under curve for the plasma concentration-time curve from administration to theoretically extrapolated infinity (AUC_{0-obx}), and the apparent volume of distribution (Vd), and the clearance rate (Cl).

52. The method of claim 50 or 51, wherein the antibody is a human or humanized antibody.

53. A method of determining effectiveness of a human or humanized antibody for the treatment of a disease, comprising
administering the antibody to the animal of any one of claims 1-23 and 33-38; and
determining the effects of the antibody on the disease.

54. The method of claim 53, wherein the disease is a tumor.

55. The method of claim 53 or 54, wherein the animal further comprises a sequence encoding a human or chimeric PD-1, PD-L1, CTLA-4, BTLA, CD27, CD28, CD40, CD47, CD137, CD154, TIGIT, TIM-3, GITR, or OX40.

56. The method of claim 55, wherein the antibody is an anti-PD-1 antibody or an anti-PD-L1 antibody.
